(19) United States
(12) Patent Application Publication
(10) Pub. No.: US 2013/0224261 A1
(43) Pub. Date: Aug. 29, 2013

(54) ENVIRONMENTALLY-RESPONSIVE NANOCOMPOSITES AND METHODS OF THEIR USE

(71) Applicant: University of Washington Through Its Center For Commercialization, Seattle, WA (US)

(72) Inventor: Hong Shen, Seattle, WA (US)

(73) Assignee: University of Washington Through Its Center For Commercialization, Seattle, WA (US)

(21) Appl. No.: 13/770,943

(22) Filed: Feb. 19, 2013

Related U.S. Application Data
(63) Continuation of application No. PCT/US2011/048497, filed on Aug. 19, 2011.
(60) Provisional application No. 61/375,229, filed on Aug. 19, 2010.

(51) Int. Cl. A61K 48/00

(52) U.S. Cl. CPC A61K 48/0008; A61K 48/0091
USPC 424/400; 514/54; 514/1.1; 514/44 R; 514/769; 435/375

(57) ABSTRACT

Environmentally-responsive composites useful for intracellular delivery of therapeutic agents.

### CROSS-REFERENCE TO RELATED APPLICATIONS

This application is a continuation of PCT/US2011/048497, filed Aug. 19, 2011, which claims the benefit of U.S. Patent Application No. 61/375,229, filed Aug. 19, 2010, each expressly incorporated herein by reference in its entirety.

### STATEMENT OF GOVERNMENT LICENSE RIGHTS

This invention was made with Government support under Grant No. 0748285 awarded by the National Science Foundation and Grant No. R21EB007494 awarded by the National Institutes of Health. The Government has certain rights in the invention.

### BACKGROUND OF THE INVENTION

Delivering DNA macromolecules to mammalian cells represents a promising therapeutic strategy and an effective tool for basic sciences. DNA molecules cannot efficiently diffuse across the intercellular and intracellular barriers alone and require the assistance of delivery vectors. Though viral-based vectors are more effective, concern over safety issues and difficulty in engineering viruses for specific cell types have limited their applications. Non-viral delivery vectors are an attractive alternative strategy due to their safety, low cost and flexibility. However, a great challenge to the material design of gene delivery systems is the heterogeneity of cell types, which exhibit distinct characteristics of transporting materials across membranes, intracellular routing, and regulation of gene expression. Though combinatorial approaches have been developed to screen a vast library of polymeric materials for gene and siRNA delivery, the usable library has so far been limited to a few number of cell types. Development of a versatile material design platform that allows customization of gene transfer with respect to an individual cell type could advance the applications of synthetic DNA delivery systems.

Strontium (Sr) shares similarities with calcium in chemical and biological characteristics and has received significant attention in the field of drug delivery and tissue engineering, in particular bone tissue engineering. Strontium ion (Sr²⁺) plays multiple roles during the course of bone regeneration. Sr²⁺ promotes pre-osteoblast differentiation and new bone formation and inhibits the osteoclast proliferation and subsequent bone resorption. Recent studies have also indicated that Sr²⁺ facilitates angiogenesis during bone regeneration.

Both strontium phosphates and carbonate apatites have been explored for gene transfer. Each is effective as calcium phosphates and poses less toxicity in primary epithelial cells. However, the method of fabricating strontium or strontium-substituted biominerals has been primarily based on spontaneous crystal formation by using supersaturated solutions or high temperature synthesis. Furthermore, the dependence of gene transfer efficiency on strontium ion content in minerals and crystallographic properties has not been studied.

Recently, a surface-induced mineralization process has been developed to fabricate DNA/calcium carbonate nanocomposites, and the resulting nanocomposites can effectively deliver DNA to bone and neural cells. The composition and morphology can be controlled and tuned through adjusting the composition of the mineral solutions and surface properties of substrates. In addition, cells can be directly grown on DNA/calcium carbonate nanocomposite-coated cell-culture-friendly surface, which permit high throughput screening of mineral formulations or substrates for a given cell type.

Despite the advances in the development of biominerals for gene transfer, a need exists for novel biomineral materials and methods for their use in intracellular therapeutic agent delivery. The present invention seeks to fulfill this need and provides further related advantages.

### SUMMARY OF THE INVENTION

In one aspect, the invention provides a colloid, comprising a plurality of substantially spherical particles, each particle comprising a strontium-containing mineral component having a therapeutic agent dispersed therein. In one embodiment, the strontium-containing mineral component has a ratio of strontium to calcium (Sr:Ca) is from about 0.2 to about 3.0. In one embodiment, the strontium-containing mineral component has a (Sr+Ca)/P value of from about 1.5 to about 2.0. Pharmaceutical compositions that include the colloid are also provided. In other embodiments, methods for delivery of a therapeutic agent to cell, methods for regulating the expression of a gene or gene product, and methods for tissue engineering using the colloid are provided.

In another aspect, the invention provides a method for making a colloid. In one embodiment, the method includes (a) combining an aqueous strontium solution with a simulated body fluid to provide a strontium-containing mineralizing solution; (b) combining the strontium-containing mineralizing solution with an aqueous therapeutic agent solution and an aqueous mineralization-inducing agent solution to provide a therapeutic agent-containing mineralizing solution; and (c) maintaining the therapeutic agent-containing mineralizing solution at a temperature and for a period of time sufficient to effect mineralization to provide a strontium-containing colloid comprising a strontium-containing mineral component having the therapeutic agent dispersed therein.

In another aspect of the invention, a substrate surface modified by mineralization is provided. The substrate surface has at least a portion of the surface has an inorganic mineral component formed thereon. In one embodiment, the inorganic mineral component comprises a therapeutic agent. In other embodiments, methods for delivery of a therapeutic agent to cell, methods for regulating the expression of a gene or gene product, and methods for tissue engineering using the substrate surface are provided.

In another aspect, a method for mineralizing a substrate surface is provided. In one embodiment, the method includes (a) combining a inorganic mineralizing solution with an aqueous therapeutic agent solution to provide a therapeutic agent-containing mineralizing solution; and (b) contacting a substrate surface with the therapeutic agent-containing mineralizing solution at a temperature and for a period of time sufficient to effect mineralization to provide a substrate surface modified by mineralization, wherein at least a portion of the surface has an inorganic mineral component formed thereon, the inorganic mineral component having a therapeutic agent dispersed therein.

In another aspect of the invention, a core-shell particle is provided. In one embodiment, the particle, comprises (a) a core comprising an inorganic mineral component; (b) a polymer shell surrounding and substantially encapsulating the core; and a therapeutic or diagnostic agent. The therapeutic or diagnostic agent can be dispersed throughout the core, dispersed throughout the shell or dispersed throughout the core and the shell. Pharmaceutical compositions that include the particles are also provided. In other embodiments, methods for delivery of a therapeutic agent to cell, methods for regulating the expression of a gene or gene product, and methods for tissue engineering using the particles are provided.

In another aspect, the invention provides a method for regulating an environmental parameter of an internal compartment of a cell, comprising contacting a cell with a colloidal particle of the invention or a core-shell particle of the invention, whereby the colloidal particle or the core-shell particle is conducted to an internal compartment of a cell where the colloidal particle or core-shell particle is solubilized and changes the parameter to a predetermined value. In certain embodiments, the parameter is pH, ionic concentration, or osmotic pressure.

In a further aspect of the invention, a particle library is provided. In one embodiment, the library comprises a plurality of particle members, each particle member being effective to regulate a microenvironment parameter to a predetermined value, wherein each particle member comprises an inorganic mineral component comprising calcium ions, potassium ions, sodium ions, phosphate ions, and chloride ions, and wherein the concentration of ions varies from member to member in a predetermined amount to provide the particle library. Representative parameters include pH, ionic concentration, and osmotic pressure.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIGS. 1A-1G compare SEM images of representative strontium-containing colloidal nanocomposites. The scale bars are 200 nm. FIG. 1H compares the size of colloidal nanocomposites before and after centrifugation.

FIG. 2 shows the chemical compositions (Sr/Ca ratio) of representative strontium-containing colloidal nanocomposites characterized by EDX.

FIGS. 3A1-3A10 compares XRD patterns of representative strontium-containing colloidal nanocomposites to standard XRD patterns of HA and β-TCP: ♦ denotes HA phase and * denotes β-TCP phase. The effect of Sr²⁺ concentration in mineralizing solution on the % of HA at 211 phase (FIG. 3B) Crystallinity of colloidal nanocomposites determined by XRD (FIG. 3C).

FIG. 4A illustrates normalized metabolic activity of MEFs cultured with representative strontium-containing colloidal nanocomposites for 24 h. The metabolic activity of cells treated with nanocomposites determined by the MTT assay was normalized to the value of cells on polystyrene surface. Data shown as means±s.e.m.

FIG. 4B illustrates the cellular uptake of YOYO-1 labeled B-DNA delivered by representative strontium-containing colloidal nanocomposites. The degree of DNA uptake was quantified as the geometric mean fluorescence intensity (GMFI) of the internalized YOYO-1 labeled DNA by flow cytometry.

FIG. 4C compares gene transfer efficiency in MEFs mediated by representative strontium-containing colloidal nanocomposites with different amount of Sr²⁺. The gene transfer efficiency of cells was expressed as the amount of the reporter enzyme β-gal normalized by total protein. Data shown as means±s.e.m. Statistical significance of differences was determined by the two-tailed Student's t-test. The gene transfer efficiency mediated by the nanocomposites were considered statistically significant different from the one without introducing DNA at *P<0.05, **P<0.01 and ***P<0.001.

FIG. 5A illustrates normalized gene transfer efficiency by intracellular level of DNA and FIG. 5B illustrates the correlation between normalized gene transfer efficiency and % HA at 211 phase.

FIGS. 6A-6F compare SEM images of surface-induced DNA-doped nanocomposites prepared from mineral solution formulations A, B, G, H, G-Sr, and G-F. The insets are EDAX spectra for the nanocomposites. Scale bars are 200 nm. FIG. 6G illustrates the kinetics of DNA precipitation. The inset illustrates the kinetics of precipitation during the first 4 h of mineralization.

FIGS. 7A-7K compare bright-field microscopy images for shows the growth of cells (MG-63, Saos-2, EMT6, Caco-2, Ishikawa, TC1, Hela, B35, Hep G2, EMT6-DOTAP, B35-DOTAP) on thin films of DNA/CaP nanocomposites prepared from mineral solution formulations A, G, G-Sr, and G-F. Cells were cultured on surfaces coated with nanocomposites formed from the indicated mineral formulation for 12 h and imaged with bright-field microscopy. Formulations B and H are not shown as nanocomposites are similar in morphology to formulation A. Cells plated on non-coated cell-culture surface (TC) are shown for comparison. The scale bar is 50 μm.

FIG. 8 compares the relative metabolic activity of cells (MG-63, Saos-2, EMT6, Caco-2, Ishikawa, TC1, Hela, B35, Hep G2) cultured on cell-culture surfaces coated with DNA-doping (prepared from mineral solution formulations A, B, G, H, G-Sr, G-F, with Lipofectamine for comparison). The metabolic activity of cells cultured on surfaces coated with nanocomposites formed from different mineral formulations was determined with the MTT assay after 36 h and normalized to the value of cells on non-coated surfaces.

FIG. 9 compares gene transfer efficiency in cells (MG-63, Saos-2, EMT6, Caco-2, Ishikawa, TC1, Hela, B35, Hep G2) by DNA-doped nanocomposites (prepared from mineral solution formulations A, B, G, H, G-Sr, G-F, with Lipofectamine for comparison). Nanocomposites were mineralized incorporating the Lac Z reporter gene encoding β-galactosidase (β-gal) for 8 h. The gene transfer efficiency of cells was expressed as the amount of the reporter enzyme β-gal normalized by total protein.

FIG. 10 compares cellular uptake of DNA mediated by cells (MG-63, Saos-2, EMT6, Caco-2, Ishikawa, TC1, Hela, B35, Hep G2) by surface-induced nanocomposites (prepared from mineral solution formulations A, B, G, H, G-Sr, G-F). Nanocomposites were mineralized with fluorescein-labeled DNA for 8 h. The degree of DNA uptake, quantified as the mean fluorescence intensity (MFI) by flow cytometry, was expressed as the MFI of cells cultured on surfaces coated with DNA-doped nanocomposites subtracted that of cells on surfaces in the absence of DNA.

FIG. 11A compares gene transfer efficiency normalized by the cellular uptake of DNA by cells (MG-63, Saos-2, EMT6, Caco-2, Ishikawa, TC1, Hela, B35, Hep G2) by surface-induced nanocomposites (prepared from mineral solution formulations A, B, G, H, G-Sr, G-F). FIG. 11B compares the pH responsiveness (pH₅₀, the pH at which 50% of calcium ions were released) of the nanocomposites (MIN: mineralization). FIG. 11C illustrates the early phagosomal pH of the cells at 15 min post-phagocytosis of 100 nm beads. FIG. 11D illustrates the correlation of the normalized gene transfer efficiency with the phagosomal pH of the cells and the pH₅₀ values of the nanocomposites. Red—the relative normalized gene transfer efficiency≧0.9; green—0.9>the relative normalized gene transfer efficiency≧0.5; blue—the relative normalized gene transfer efficiency<0.5. The relative normalized gene transfer efficiency is the normalized gene transfer efficiency divided by highest normalized gene transfer efficiency of that cell type among different mineral formulations. The dashed line is for equal pH. Values shown are the mean of the triplicates with a standard deviation less than 5% of the mean.

FIGS. 12A-12C compare fluorescent microscopy images of DNA-doped nanocomposites: fluorescein-labeled DNA co-precipitated with mineral solution formulation A for 8 h and imaged (12A); fluorescein-labeled DNA was first complexed with DOTAP at a DNA:DOTAP ratio (w/w) with mineral solution formulation A (12B) 1:6 or 1:12 (12C), mineralized for 8 h, and imaged. The scale bar is 50 μm.

FIG. 13A compares the pH₅₀ (pH sensitivity) of modified DNA/DOTAP-doped nanocomposites (prepared from mineral solution formulations A, B, G, H, G-Sr (Sr), G-F (F)) in which the DNA was complexed with DOTAP at DNA:DOTAP ratios (w/w) of 1:6 and 1:12 (MIN:mineralization). FIG. 13B compares the DNA precipitation efficiency of the DNA/DOTAP-doped nanocomposites.

FIGS. 14A-14C compare gene transfer in EMT6 and B35 cells on DOTAP/DNA nanocomposite-coated substrates. FIG. 14A compares cellular uptake of DNA: fluorescein-labeled DNA was complexed with DOTAP at a DNA:DOTAP ratio (w/w) of 1:6 for EMT6 and 1:12 for B35 cells, respectively. DNA uptake was quantified by flow cytometry and expressed as the MFI of cells cultured on nanocomposites subtracted the MFI of control cells without DNA. The uptake for cells on unmodified nanocomposites is shown for comparison. FIG. 14B compares gene transfer efficiencies: the LacZ reporter gene was complexed with DOTAP at a DNA:DOTAP ratio (w/w) of 1:6 for EMT6 and 1:12 for B35 cells, respectively. The formed complex was mineralized with different mineral formulations for 8 h and the gene transfer efficiency was evaluated. EMT6 and B35 are the gene transfer efficiencies for cells on unmodified nanocomposites. FIG. 14C compares metabolic activity of cells. Values shown are the mean of the triplicates with a standard deviation less than 5% of the mean.

FIGS. 15A-15D show the characterization of DOTAP liposomes and lipoplexes. The DOTAP liposomes were prepared using the direct mixing and freeze-thaw-extrusion methods. Sizes of DOTAP liposomes or lipoplexes made by (FIG. 15A) the direct mixing method, and by the freeze-thaw-extrusion method with (FIG. 15B) 100 nm filter or (FIG. 15C) 1000 nm filter. (FIG. 15D) DNA complexation efficiency. The DNA:DOTAP ratio was 1:12 (w/w).

FIG. 16 shows the DNA immobilization efficiency in the form of lipoplexes at 8 h by surface-induced biomineralization.

FIG. 17A shows SEM images of surface-induced nanocomposites formed from the indicated mineral formulation (A, B, G, H, G-Sr, G-F). The scale bar is 200 nm.

FIG. 17B shows surface plots of the fluorescence intensity of FITC-labeled DNA on the surfaces deposited with DNA- or lipoplex-nanocomposites.

FIG. 18A shows morphology and FIG. 18B the relative metabolic activities of B35 cells cultured on lipoplex-nanocomposites formed from the indicated mineral formulation (A, B, G, H, G-Sr, G-F). The cells on tissue culture-treated (TC) surface were used as comparison. The scale bar is 50 μm. The DNA:DOTAP ratio was 1:12 (w/w).

FIG. 19 shows cellular DNA uptake mediated by surface-induced lipoplex-nanocomposites formed from mineral formulations A, B, G, H, G-Sr and G-F. Naked DNA-doped nanocomposites (Naked-DNA), free lipoplexes made from DOTAP (DOTAP) were used as comparison. The DOTAP liposomes were prepared using freeze-thaw-extrusion method with 100 nm or 1000 nm filter. Fluorescein-labeled DNA was used. The ratio of DNA to DOTAP was 1:12 (w/w).

FIGS. 20A-20E compare gene transfer efficiency of B35 cells medicated by surface-induced lipoplex—nanocomposites. FIGS. 20A and 20B: the effect of complexation duration. DNA:DOTAP ratio was 1:12 (w/w). FIGS. 20C and 20D: the effect of DNA:DOTAP ratios. The complexation duration was 15 min. The DOTAP liposomes were prepared using freeze-thaw-extrusion method with (FIGS. 20A and 20C) 100 nm or (FIGS. 20B and 20D) 1000 nm filter. Naked DNA-doped nanocomposites (Naked-DNA), free lipoplexes made from DOTAP (DOTAP) were used as controls. FIG. 20E: lipoplexes were made from Lipofectamine™ 2000. The DNA were complexed with Lipofectamine™ 2000 at different ratios and the complexation duration was 20 min.

FIG. 21 shows the fabrication of polymer templates and patterning of mineral nanostructures on PLGA mesospheres.

FIG. 22A-22D show SEMs and TEMs of unmodified PLGA mesospheres (FIGS. 22A and 22B) and mesospheres mineralized with SBF (FIGS. 22C and 22D). Scale bars in SEMS are 1 μm. Scale bars in TEMs are 50 nm and 100 nm, respectively.

FIGS. 23A-23D show TEMs of PLGA mesospheres with no tilt (FIG. 23A) and tilted 22.5° (FIG. 23B). Calculated the radial position, R, of all the quantum dots shown in TEMs and obtained R=148.0±3.6 nm. The radius of the mesosphere in FIGS. 23A and 23B was 145.0 nm. All the quantum dots are situated on the surface of the mesosphere. The scale bars in TEMs are 50 nm. Calculations were performed as below:

FIGS. 24A-24D show SEMs of PLGA mesospheres with NaOH treatment but no quantum dots (FIG. 24A), silver colloids (FIG. 24B), gold colloids (FIG. 24C), and TOPO after mineralization (FIG. 24D). The scale bars in SEMs are 1 μm.

FIGS. 25A-25D show SEMs of mesospheres mineralized with SBF in the presence of 0%, 0.2%, 1%, and 2% PEG, respectively. Scale bars are 1 μm.

FIG. 26A shows SEM of mesospheres mineralized with SBF-G and FIG. 26B are EDX spectra of mesospheres mineralized with SBF and SBF-G. PLGA mesospheres with and without quantum dots are shown as controls. Scale bars are 1 μm.

FIG. 27A shows viability of cells exposed to nanocomposites or Lipofectamine (Lipo); FIG. 27B shows efficiency of gene transfer in nine cell types. Legends on x-axis are the different cell types derived from bone, breast, intestine, reproductive, lung and neural and liver tissues. All cells are cell line specimens.

FIGS. 28A-28C compares cytokines induction by macrophages exposed to nanocomposites (A, B, G, H, G-Sr, G-F) and Lipofectamine (Lipo).

FIGS. 29A-29C show the reduction in gene transfer efficiency in TC-1 cells being exposed to the supernatants, which were collected from macrophages exposed to nanocomposites G, Lipofectamine and PEI for 24 h. The data is represented as the reduction compared to the gene transfer efficiency of TC-1 exposed to fresh cell culture medium.

FIG. 30 compares the efficiency of siRNA delivered by nanocomposite G in suppressing c-myc gene expression in different cell types compared to Lipofectamine (Lipo). For some cell types, nanocomposite G is more efficient than Lipofectamine.

### DETAILED DESCRIPTION OF THE INVENTION

The invention provides environmentally-responsive composites and methods for making and using the composites. The composites can be prepared from biomineralizing solutions by mineralization. In certain embodiments, the composite is in the form of particles. In other embodiments, surfaces are prepared having the composite formed thereon. In certain embodiments, the biomineralizing solution is formulated based on the composition of blood plasma. The composites can include one or more therapeutic agents that are useful for intracellular delivery of therapeutic agents. In certain embodiments, the composites advantageously include therapeutic agents that need intracellular delivery for the therapeutic agent to be effective.

Biomineral-Containing Nanocomposites and Colloids

The invention provides environmentally-responsive composites that can be prepared from biomineralizing solutions. In such embodiments, the composites are biomineral-containing composites. Thus, in certain aspects, the invention provides biomineral-containing nanocomposites, biomineral-containing colloidal particles, compositions that include the nanocomposites and colloidal particles, and methods for making and using the nanocomposites and colloidal particles.

In one aspect, the invention provides a colloid that includes substantially spherical particles. The particles of the colloid include a biomineral-containing component. In one embodiment, the biomineral-containing component is a strontium-containing mineral component. In certain embodiments, the biomineral-containing component further includes one or more of magnesium and fluoride. In certain embodiments, the biomineral-containing component has a therapeutic agent dispersed therein.

The biomineral-containing component includes strontium, calcium, potassium, sodium, phosphate, and chloride ions. In one embodiment, the biomineral-containing component is a strontium-containing nanocomposite.

Representative colloid particles have strontium-containing mineral components that have a ratio of strontium to calcium (Sr:Ca) is from about 0.2 to about 3.0. Representative colloid particles have strontium-containing mineral components that have strontium-containing mineral components that have a (Sr+Ca)/P value of from about 1.5 to about 2.0. In certain embodiments, representative colloid particles have strontium-containing mineral components that have a ratio of strontium to calcium (Sr:Ca) is from about 0.2 to about 3.0 and a (Sr+Ca)/P value of from about 1.5 to about 2.0.

Representative colloid particles have strontium-containing mineral components that have a strontium-containing mineral component has a ratio of strontium to phosphorus (Sr:P) is from about 0.3 to about 1.5. Representative colloid particles have strontium-containing mineral components that have a ratio of calcium to phosphorus (Ca:P) is from about 0.5 to about 1.0.

In certain embodiments, the colloid particles have strontium-containing mineral components having a percent crystallinity up to about 95%. In other embodiments, the colloid particles have strontium-containing mineral components having a percent crystallinity from about 80 to about 90%. In certain embodiments, the colloid particles have strontium-containing mineral components having an orientation of crystal growth along the hydroxyapatite 211 plane that is up to about 90%. In other embodiments, the colloid particles have strontium-containing mineral components having an orientation of crystal growth along the hydroxyapatite 211 plane from about 80 to about 90%.

Representative colloid particles have a diameter of from about 100 nm to about 10 μm and a polydispersity index of from about 0.1 to about 0.5. As used herein, the term “polydispersity index” refers to the size distribution of the particle. In certain embodiments, the colloid particles are substantially monodispersed. As used herein, the term “substantially monodispersed” refers to particles that have a polydispersity index of less than about 0.3.

Representative colloid particles include one or more therapeutic agents dispersed therein. In certain embodiments, the therapeutic agent is a biopolymer. Representative biopolymers include nucleic acids, polysaccharides, peptides, polypeptides, proteins, and fragments thereof. Representative nucleic acids include DNAs and RNAs. In certain embodiments, the therapeutic agent is a therapeutic small molecule. Representative therapeutic small molecules include chemotherapeutic agents and antimicrobial agents.

In another aspect of the invention, a pharmaceutical composition useful for administration of a therapeutic agent is provided. The pharmaceutical composition includes a pharmaceutically acceptable carrier and a colloid of the invention having a therapeutic agent dispersed therein.

In other aspects, the invention provides methods for using the colloids of the invention.

In one embodiment, the invention provides a method for delivering a therapeutic agent to cell. In the method, a cell is contacted with a colloid of the invention having a therapeutic agent dispersed therein.

In another embodiment, the invention provides a method for regulating the expression of a gene or gene product. In the method, a cell is contacted with a colloid of the invention having dispersed therein a DNA or RNA effective to regulate the expression of the gene or gene product.

In a further embodiment, the invention provides a method for tissue engineering. In one embodiment of the method, cells of interest (e.g., stem cells) are mixed with a colloid of the invention having a nucleic acid or a protein dispersed therein. In another embodiment, the colloid of the invention having a nucleic acid or a protein dispersed therein is coated on a surface used for tissue implants.

In a further aspect, the invention provides methods for making a biomineral-containing colloid. In one embodiment, the method for making a biomineral-containing colloid includes (a) combining an aqueous strontium solution with a simulated body fluid to provide a strontium-containing mineralizing solution; (b) combining the strontium-containing mineralizing solution with an aqueous therapeutic agent solution and optionally an aqueous mineralization-inducing agent solution to provide a therapeutic agent-containing mineralizing solution; and (c) maintaining the therapeutic agent-containing mineralizing solution at a temperature and for a period of time sufficient to effect mineralization to provide a strontium-containing colloid comprising a strontium-containing mineral component having the therapeutic agent dispersed therein.

In one embodiment, the method further includes collecting the strontium-containing mineral component (i.e., colloid particles). The strontium-containing mineral component colloid can be collected by concentrating the mineral component by removing some or all of the residual solution. In one embodiment, the method further includes re-suspending the collected strontium-containing mineral component in an aqueous medium to provide a second strontium-containing colloid.

In certain embodiments, the aqueous strontium solution has a strontium concentration of from about 0.1 to about 20 mM. In one embodiment, the aqueous strontium solution has a strontium concentration of from about 1 to about 10 mM. In certain embodiments, the aqueous strontium solution comprises an aqueous solution of strontium (II) chloride.

The simulated body fluid can be prepared from calcium chloride, potassium dihydrogen phosphate, sodium chloride, potassium chloride, and sodium bicarbonate at concentrations sufficient to mimic the plasma concentrations of calcium, potassium, sodium, phosphate, and chloride ions. The simulated body fluid therefore includes calcium, potassium, sodium, phosphate, and chloride ions.

The aqueous therapeutic agent solution includes one or more therapeutic agents. In certain embodiments, the therapeutic agent is a biopolymer, such a nucleic acid, a polysaccharide, a peptide, a polypeptide, a protein, or fragments thereof. Representative nucleic acids include DNAs and RNAs. In certain embodiments, the therapeutic agent is a therapeutic small molecule. Representative therapeutic small molecules include chemotherapeutic agents and antimicrobial agents.

The aqueous mineralization-inducing agent solution includes a mineralizing-inducing agent (e.g., nucleation-inducing agent). Suitable mineralizing-inducing agents have carboxylic acid (COOH) or carboxylate (COO⁻), hydroxyl (OH), amine (NH₂ or NH₃), sulfate (SO₃⁻), or phosphate (PO₄⁻) groups. Representative mineralizing-inducing agents include polyethylene glycols, polypeptides, peptides, and nucleic acids. Representative polyethylene glycols have a number average molecule weight from about 2 to about 40 kDa (e.g., 4, 10, 20 kDa). The aqueous mineralization-inducing agent solution includes a mineralizing-inducing agent at concentration of about 0.5 to about 5 percent w/v. In one embodiment, the aqueous mineralizing-inducing solution is a 2% w/v solution of a polyethylene glycol (20 kDa).

The preparation, characteristics, and methods for using representative strontium-containing colloids of the invention are described in Example 1.

The following is a description of representative strontium-containing colloidal nanocomposites and methods for their use.

Characterization of Colloidal Nanocomposites.

Representative nanocomposites were induced by mixing DNA and poly(ethylene glycol) (PEG) with mineralizing solutions containing different concentrations of strontium ions (Table 1) at 37° C. The mineralizing solutions were based on simulated body fluid (SBF) by replacing magnesium ions with strontium ions (Sr²⁺). Though the exact mechanism by which PEG controls the mineralization is still unclear, PEG may be able to chelate Ca²⁺ to form PEG-O—Ca²⁺-O-PEG bonds, which may react with PO₄³⁺ to form hydroxyapatite. PEG has recently been successfully used to formulate siRNA-containing nanocomposites. The concentration and molecular weight of PEG in mineralizing solutions was 20 kDa PEG at 2% (w/v).

TABLE 1
Formulation of representative mineral solutions.

| | Sr²⁺ concentration (mM) | | | | | | |
|---|---|---|---|---|---|---|---|
| | 0.25 | 0.5 | 1 | 3 | 5 | 10 | 20 |
| CaCl₂•H₂O | 2.5 | 2.5 | 2.5 | 2.5 | 2.5 | 2.5 | 2.5 |
| KH₂PO₄ | 1 | 1 | 1 | 1 | 1 | 1 | 1 |
| NaCl | 141 | 141 | 141 | 141 | 141 | 141 | 141 |
| KCl | 4 | 4 | 4 | 4 | 4 | 4 | 4 |
| NaHCO₃ | 4.2 | 4.2 | 4.2 | 4.2 | 4.2 | 4.2 | 4.2 |
| SrCl₂ | 0.25 | 0.5 | 1 | 3 | 5 | 10 | 20 |

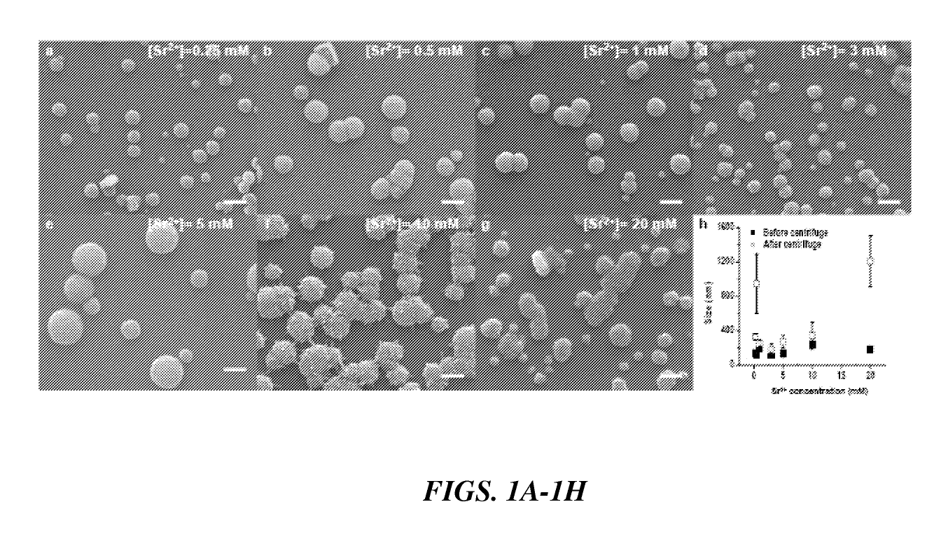

The size of colloidal nanocomposites was examined by dynamic light scattering (DLS) and scanning electron microscope (SEM). Size measurements from both DLS and SEM were consistent. Representative nanocomposites were 120-240 nm in diameter with a polydispersity index (PDI) of 0.2-0.5 (Table 2, FIG. 1). There was no distinct size dependence on the concentration of strontium ions in mineralizing solutions. However, the colloidal stability of nanocomposites was significantly affected by the concentration of strontium ions. When colloidal nanocomposites were concentrated by centrifugation, nanocomposites formed at intermediate strontium ion concentration (1 to 10 mM) remained mono-dispersed and similar in size to those before centrifugation. Significant aggregation was observed for nanocomposites formed at both low and high strontium ion concentrations (FIG. 1H).

TABLE 2
Size and PDI of representative strontium-containing colloidal
nanocomposites.

| Sr²⁺ concentration (mM) | Size (nm) | PDI |
|---|---|---|
| 0.25 | 140.1 ± 6.3 | 0.25 |
| 0.5 | 123.0 ± 11.4 | 0.2 |
| 1 | 197.4 ± 16.2 | 0.29 |
| 3 | 126.6 ± 12.3 | 0.24 |
| 5 | 130.3 ± 4.0 | 0.24 |
| 10 | 242.9 ± 19.8 | 0.42 |
| 20 | 183.4 ± 32.2 | 0.49 |

The morphology of representative strontium-containing colloidal nanocomposites was examined using SEM (FIGS. 1A-1G). The nanocomposites adopted spherical morphologies. The surface of the nanocomposites became rough with the formation of spiky secondary structures when the strontium ion concentration was at 10 mM. Similar size and morphology were observed in the presence and absence of DNA. Therefore, the effect of DNA on the formation of nanocomposites was insignificant within the concentration of DNA (<5 μg/ml). DNA (100%) was incorporated into nanocomposites regardless of strontium ion concentration.

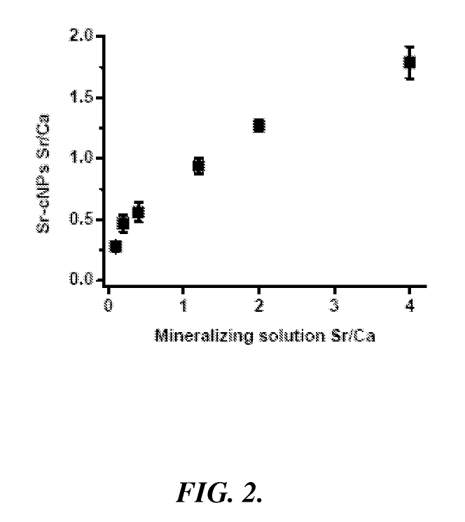

Energy dispersive X-ray spectroscopy (EDX) was used to examine the composition of key elements, including calcium, strontium, and phosphorus, in the strontium-containing colloidal nanocomposites. The Sr/Ca ratio in the nanocomposites linearly increased with the increase of Sr²⁺ concentration in the mineralizing solution (FIG. 2, Table 3). The ratio of (Sr+Ca)/P was maintained at 1.6±0.1 to 1.95±0.23, which is close to stoichiometric ratio of Ca/P (1.67) of hydroxyapatite (HA). This suggests that the calcium ions in the HA lattice were replaced by strontium ions. The increase of Sr²⁺ concentration in mineralizing solution led to increased incorporation of strontium ions and correspondingly decreased incorporation of calcium ions into nanocomposites.

TABLE 3
Chemical compositions of representative strontium-containing
colloidal nanocomposites as determined by EDX analysis.

| Sr²⁺ | | | | |
|---|---|---|---|---|
| Concentration | | | | |
| (mM) | Sr:P | Ca:P | Sr/Ca | (Sr + Ca)/P |
| 0.25 | 0.38 ± 0.06 | 1.38 ± 0.10 | 0.28 ± 0.04 | 1.76 ± 0.12 |
| 0.5 | 0.52 ± 0.10 | 1.10 ± 0.06 | 0.47 ± 0.07 | 1.62 ± 0.14 |
| 1 | 0.57 ± 0.09 | 1.03 ± 0.02 | 0.56 ± 0.08 | 1.60 ± 0.10 |
| 3 | 0.74 ± 0.08 | 0.78 ± 0.09 | 0.94 ± 0.06 | 1.52 ± 0.16 |
| 5 | 1.02 ± 0.14 | 0.80 ± 0.09 | 1.27 ± 0.04 | 1.82 ± 0.23 |
| 10 | 1.06 ± 0.15 | 0.59 ± 0.04 | 1.79 ± 0.13 | 1.65 ± 0.19 |
| 20 | 1.44 ± 0.18 | 0.51 ± 0.04 | 2.85 ± 0.15 | 1.95 ± 0.23 |

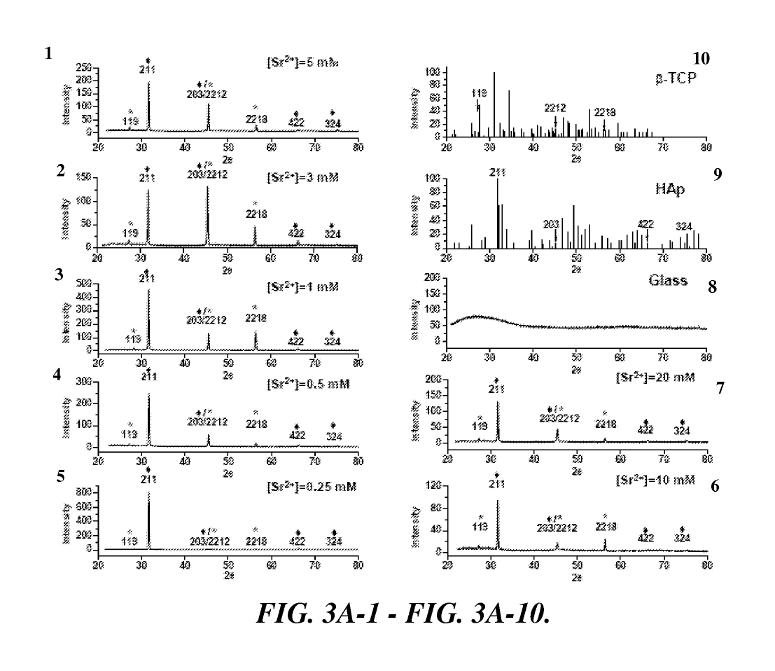

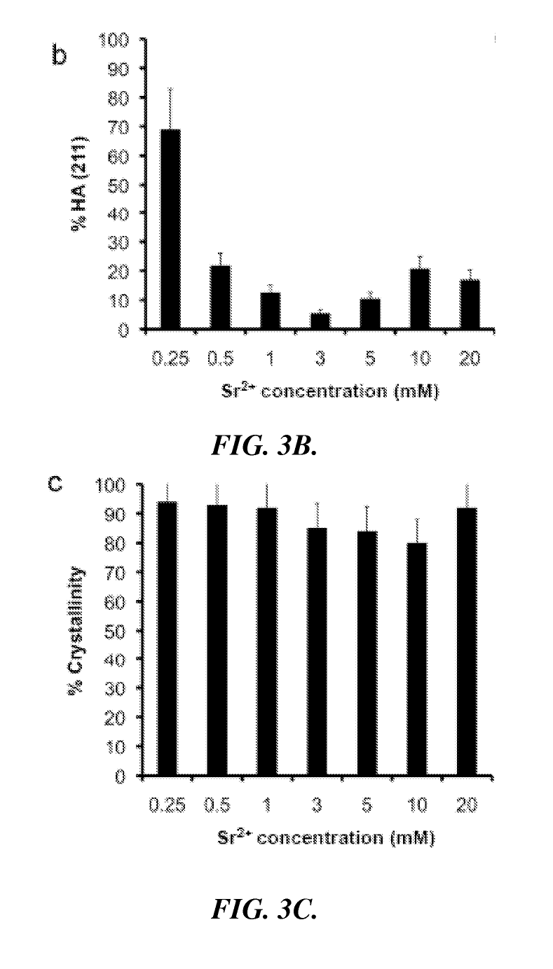

X-Ray Diffraction (XRD) was used to determine crystallographic properties of the colloidal nanocomposites. The XRD pattern was compared with the diffraction pattern of standard crystalline hydroxyapatite (HA), α-tricalciumphosphate (α-TCP), β-tricalciumphosphate (β-TCP), octacalcium phosphate (OCP), carbonated HA, and Sr-HA. The nanocomposites consisted primarily of HA and β-TCP though predominant crystallographic orientations varied with Sr²⁺ content (FIG. 3). The dominant crystallographic orientations were at 31.75°, 45.45°, and 56.4° (FIG. 3A). The diffraction at 31.75° was from the 211 plane of the HA phase while the diffraction at 56.4° was from β-TCP. Diffraction at 45.45° may be from the 203 plane of HA or from the (2,2,12) plane of β-TCP because both HA and β-TCP exhibited scattering at 45.306°. It has been suggested that Sr ions are not readily incorporated in the lattice of β-TCP. The slight shift from 45.306° to 45.45° indicates Sr²⁺ may replace Ca²⁺, which likely occurred in HA phase, but not β-TCP phase.

The percentage of HA at the 211 plane was correlated with Sr²⁺ content to provide a semi-quantitative understanding of how Sr²⁺ content affects the crystal orientation and phases under mineralizing conditions (FIG. 3B). At lower Sr²⁺ content, the crystal preferred growth was along the 211 plane. As the Sr²⁺ content increased, the crystal started to grow along other directions and the β-TCP phase started to appear. As the Sr²⁺ content further increased, the crystal growth showed an increasing preference over the 211 plane of HA.

Both crystallinity and crystallite sizes decreased with the increase of Sr²⁺ content when the Sr²⁺ concentration was less than 20 mM in mineralizing solutions (FIG. 3C).

The compositional and structural properties of the strontium-containing colloidal nanocomposites was controlled by the concentration of Sr²⁺ in mineralizing solutions.

Biocompatibility, Cellular Uptake and Gene Transfer Mediated by Sr Colloidal Nanocomposites.

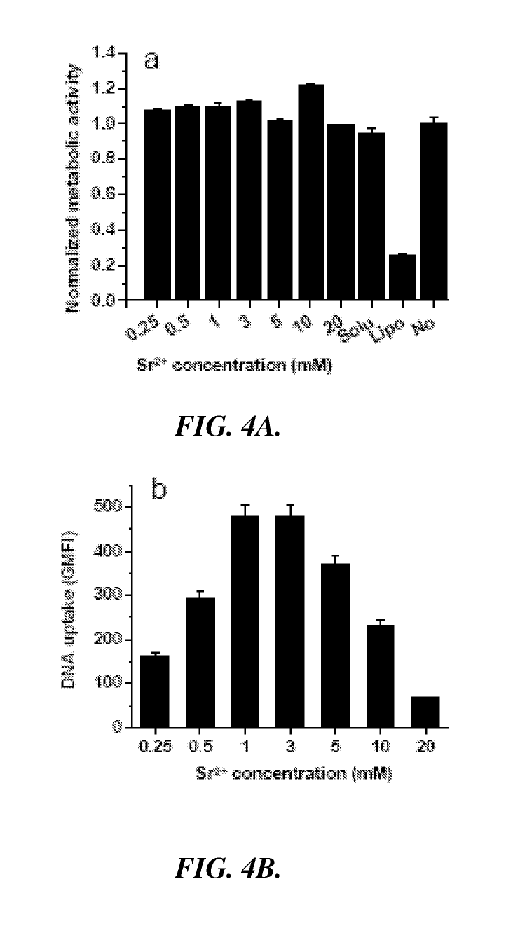

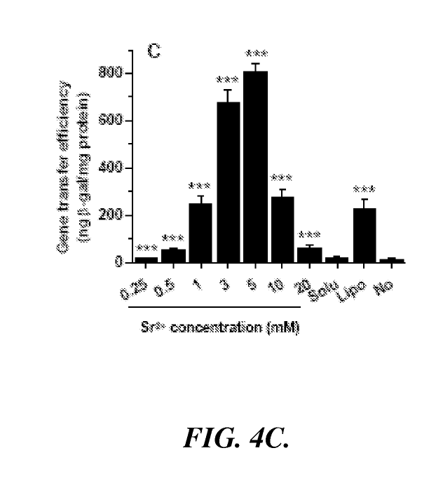

Representative nanocomposites concentrated by centrifugation were used for evaluating toxicity, cellular uptake, and gene transfer. The cytotoxicity of the nanocomposites was evaluated by comparing the metabolic activity of cells treated with strontium colloidal nanocomposites to that of cells without any treatment. MEFs treated with strontium nanocomposites proliferated as well as cells without any treatment. There was no cell death observed at the concentration of nanocomposites used (FIG. 4A). Previous studies have shown that strontium phosphates and carbonate apatites pose less toxicity on many epithelial cell types compared to calcium phosphates.

The intracellular level of DNA was highest at intermediate Sr²⁺ content and decreased significantly at both high and low Sr²⁺ content (FIG. 4B). This trend is inversely correlated with the average size of nanocomposites after centrifugation (FIG. 1A). The cellular uptake of particulates is mainly affected by size, charge, and shape in particulate systems. The charge and shape of nanocomposites with different Sr²⁺ content were similar. The effect of Sr²⁺ content on cellular uptake of nanocomposites was mainly due to their effect on the colloidal stability of nanocomposites, and the size of nanocomposites upon centrifugation.

The ability of strontium-containing colloidal nanocomposites to mediate the gene transfer was examined in primary cells derived from fetal mouse skin, MEFs (FIG. 4C). The gene transfer efficiency was dependent on Sr²⁺ content in the nanocomposites. Significantly higher gene transfer efficiency was obtained at intermediate level of Sr²⁺ content than at either lower or higher Sr²⁺ contents. The greatest gene transfer efficiency (804±40 ng β-gal/mg protein) was obtained using nanocomposites made from 5 mM Sr²⁺. This level was 3.5 fold greater than that of the commercial reagent, Lipofectamine 2000™.

Correlation of Crystallographic Properties with Gene Transfer Efficiency Mediated By Sr Nanocomposites.

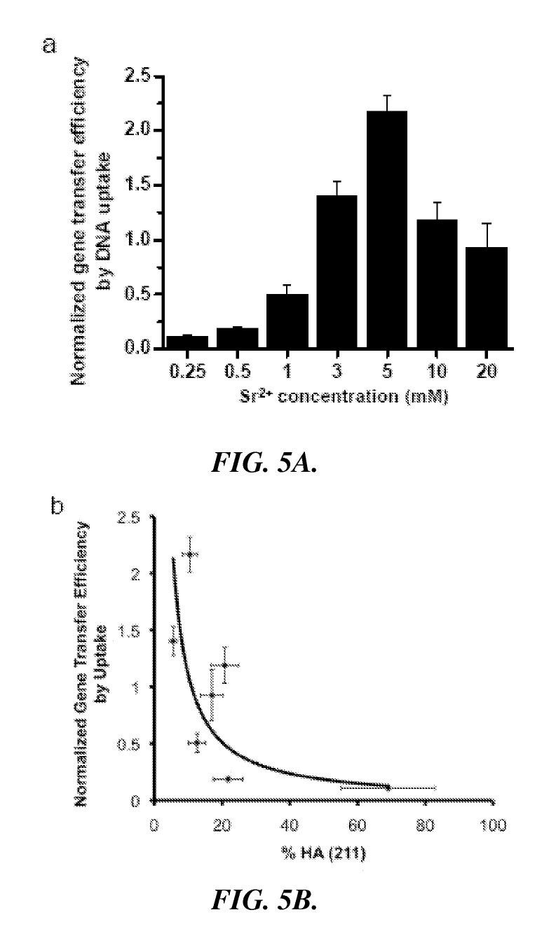

The dependence of gene transfer efficiency on Sr²⁺ content can be attributed to the effect of Sr²⁺ content on the level of internalized DNA and/or the dissolution of nanocomposites in response to cellular environmental change, in particular the change of pH and ion concentrations. To exclude the effect of intracellular level of DNA, the transgene expression level was first normalized based on the intracellular DNA level. As shown in FIG. 5A, the normalized transgene expression still exhibited a strong dependence on Sr²⁺ content. This suggests that the dissolution of internalized nanocomposites has an effect on the gene transfer efficiency as well. The solubility of HA is lower than β-TCP. Additionally, the incorporation of Sr²⁺ into HA increases the solubility of HA in an acidic environment. Sr²⁺ content did not significantly affect the crystallite size and the crystallinity of nanocomposites prepared as described herein, but did affect the orientation of crystal growth and crystal phases (FIG. 3). These results suggest that there may be a correlation between crystallinity/crystal orientation/phase and the gene transfer efficiency. The normalized gene transfer efficiency was correlated with the percentage of HA at 211 plane (FIG. 5B). The normalized transgene expression was inversely proportional to the increase of the percentage of HA at 211 plane. HA is a stable phase at pH 5 at 100 mM KCl. Upon internalization, nanocomposites with high HA content can be stable at endosomal pH (5 to 6) at the ion compositions close to simulated body fluid. As a result, DNA may not be effectively released from nanocomposites for subsequent transcription and translation.

The present invention provides a facile process to fabricate well-dispersed spherical strontium-containing biominerals (nanocomposites) through the use of mineralizing solutions (e.g., derived from simulated body fluid in the presence of poly(ethylene glycol)). The mineralization proceeded at 37° C. and neutral pH, permitting the incorporation and preservation of biological molecules, such as DNA. The content of strontium ions in biominerals are tunable through manipulating the composition of mineralizing solutions. The content of strontium ions in the biominerals define the crystallinity and predominant crystallographic orientation of the nanocomposites. In mouse embryonic fibroblasts (MEFs), gene transfer efficiency correlated with the content of strontium ion and crystallographic properties of the nanocomposites. The close correlations between composition, structural properties, and functions offer the ability to control biological functions by biomaterials. The strontium-containing colloidal nanocomposites can be used for gene delivery and tissue engineering as either injectable formulations or coatings on tissue implants.

Mineralized Substrate Surfaces and Surface-Induced Mineralization

The invention provides mineralized substrate surfaces, surface-induced mineralization methods, and methods for using mineralized substrate surfaces.

In one aspect, the invention provides a substrate surface modified by mineralization (i.e., a mineralized substrate surface). In one embodiment, at least a portion of the surface has an inorganic mineral component formed thereon. In certain embodiments, the inorganic mineral component has a therapeutic agent dispersed therein.

The inorganic mineral component can be prepared from calcium chloride, potassium dihydrogen phosphate, sodium chloride, potassium chloride, and sodium bicarbonate at concentrations sufficient to mimic the plasma concentrations of calcium, potassium, sodium, phosphate, and chloride ions (e.g., simulated body fluid). The inorganic mineral component therefore includes calcium, potassium, sodium, phosphate, and chloride ions. In certain embodiments, the inorganic mineral component includes one or more ions selected from magnesium ions (e.g., magnesium sulfate and/or magnesium chloride), strontium ions (e.g., strontium chloride), and fluoride ions (e.g., sodium fluoride).

Representative substrate surfaces optionally include one or more therapeutic agents dispersed therein. In certain embodiments, the therapeutic agent is a biopolymer. Representative biopolymers include nucleic acids, polysaccharides, peptides, polypeptides, proteins, and fragments thereof. Representative nucleic acids include DNAs and RNAs. In certain embodiments, the therapeutic agent is a therapeutic small molecule. Representative therapeutic small molecules include chemotherapeutic agents and antimicrobial agents.

Representative substrate surfaces include polymers used for implants (e.g., poly(lactic-co-glycolic acid), collagen, chitosan, polyethylene), glass (e.g., silicon dioxide, bioglass), carbon (e.g., carbon nanotubes), plastic, metal (e.g., stainless steel, gold, titanium), metal alloy (e.g., titanium alloys, cobalt-chromium alloys, aluminum oxide, zirconium oxide), and ceramic surfaces. Surfaces having a mixture of surface types are also useful. Suitable surfaces have carboxylic acid (COOH) or carboxylate (COO⁻), hydroxyl (OH), amine (NH₂ or NH₃), sulfate (SO₃⁻), or phosphate (PO₄⁻) functional groups. In one embodiment, the substrate is an implantable device such as tissue engineering scaffolds and synthetic implants (e.g., hip replacements, artificial bone, cartilage).

In other aspects, the invention provides methods for using the mineralized substrate surfaces of the invention.

In one embodiment, the invention provides a method for delivering a therapeutic agent to cell. In the method, a cell is contacted with a substrate surface of the invention having a therapeutic agent dispersed therein.

In another embodiment, the invention provides a method for regulating the expression of a gene or gene product. In the method, a cell is contacted with a substrate surface of the invention having dispersed therein a DNA or RNA effective to regulate the expression of the gene or gene product.

In a further aspect, the invention provides methods for mineralizing a substrate surface. In one embodiment, the method includes (a) combining an inorganic mineralizing solution (e.g., simulated body fluid) with an aqueous therapeutic agent solution to provide a therapeutic agent-containing mineralizing solution; and (b) contacting a substrate surface with the therapeutic agent-containing mineralizing solution at a temperature and for a period of time sufficient to effect mineralization to provide a substrate surface modified by mineralization, wherein at least a portion of the surface has an inorganic mineral component formed thereon, the inorganic mineral component having a therapeutic agent dispersed therein.

The inorganic mineralizing solution can be prepared from calcium chloride, potassium dihydrogen phosphate, sodium chloride, potassium chloride, and sodium bicarbonate at concentrations sufficient to mimic the plasma concentrations of calcium, potassium, sodium, phosphate, and chloride ions (e.g., simulated body fluid). The inorganic mineral component therefore includes calcium, potassium, sodium, phosphate, and chloride ions. In certain embodiments, the inorganic mineralizing solution further includes one or more ions selected from magnesium ions (e.g., magnesium sulfate and/or magnesium chloride), strontium ions (e.g., strontium chloride), and fluoride ions (e.g., sodium fluoride).

The aqueous therapeutic agent solution includes one or more therapeutic agents. In certain embodiments, the therapeutic agent is a biopolymer, such as described above. In other embodiments, the therapeutic agent is a therapeutic small molecule, such as described above.

Representative substrates and substrate surfaces include those noted above.

The preparation, characteristics, and methods for using mineralized substrate surfaces of the invention are described in Example 2.

The following is a description of representative mineralized substrate surfaces, surface-induced mineralization methods, and related gene delivery systems.

Mineralization solution compositions for making representative mineralized surfaces useful the methods of the invention are shown in Table 4.

TABLE 4
Mineral solution compositions.

| Concentration (mM) | A | B | G | H | G-Sr | G-F |
|---|---|---|---|---|---|---|
| CaCl₂•2H₂O | 2.5 | 3.7 | 2.5 | 2.5 | 2.5 | 2.5 |
| KH₂PO₄ | 1 | 1 | 1 | 1 | 1 | 1 |
| NaCl | 141 | 141 | 141 | 141 | 141 | 141 |
| KCl | 4 | 4 | 4 | 4 | 4 | 4 |
| MgSO₄•6H₂O | 0.5 | 0.5 | 0 | 0.5 | 0 | 0 |
| MgCl₂•6H₂O | 1 | 1 | 0 | 5 | 0 | 0 |
| NaHCO₃ | 4.2 | 4.2 | 4.2 | 4.2 | 4.2 | 4.2 |
| SrCl₂ | 0 | 0 | 0 | 0 | 3 | 0 |
| NaF | 0 | 0 | 0 | 0 | 0 | 3 |

Morphology and Composition of Surface-Induced Calcium Carbonate Nanocomposites.

A library of six mineral solutions was evaluated based on variations from simulated body fluid (Table 4). In G-Sr, strontium (Sr) was added to mineral solution G. In mineral solution G-F, fluoride (F) was added to mineral solution G.

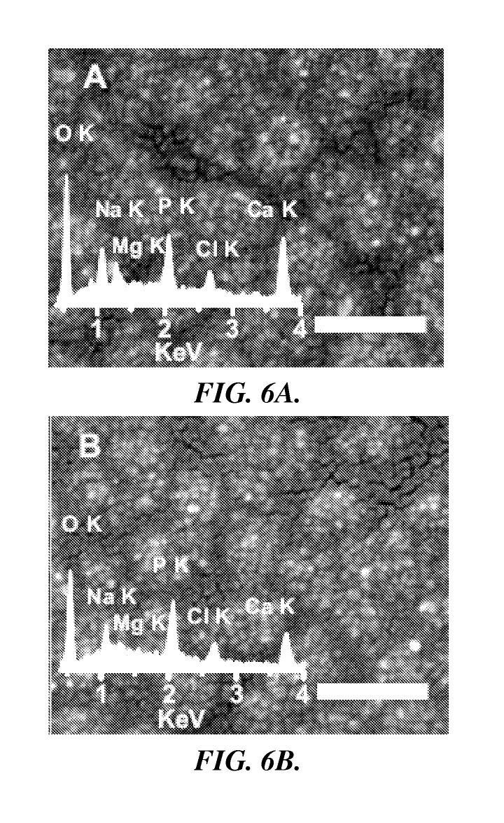

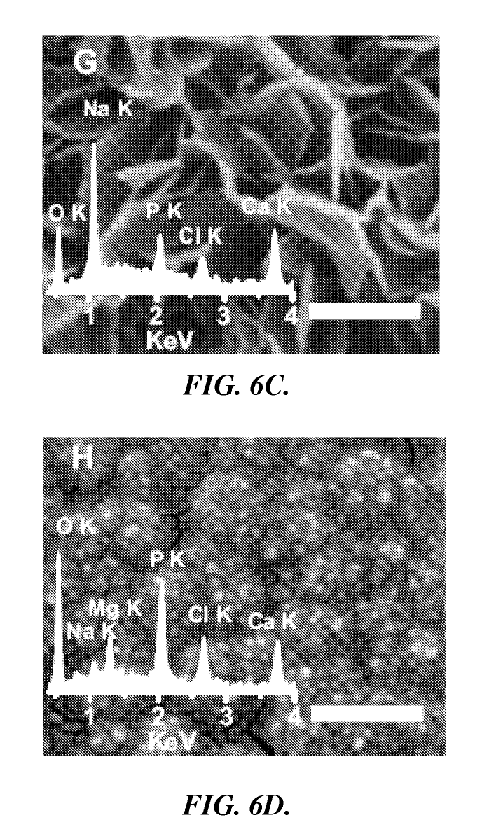

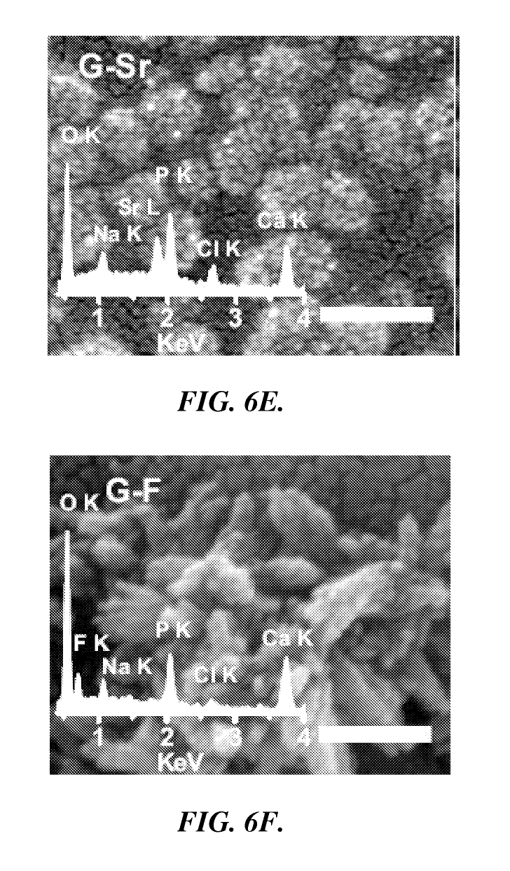

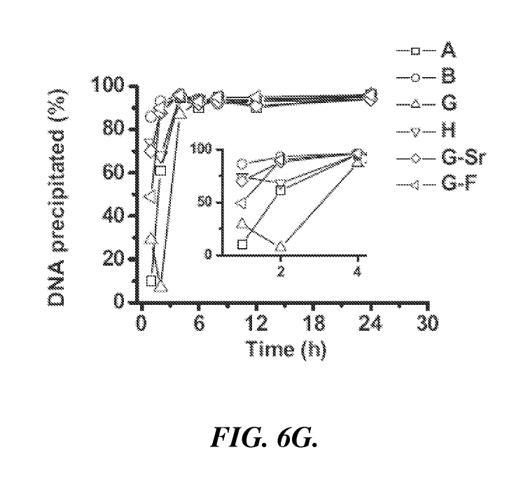

The mineralization was initiated on tissue culture-treated polystyrene surfaces. Nanocomposites were identified by the mineral composition from which they were derived. Scanning electron microscopy (SEM) was used to examine the morphology of nanocomposites. As shown in FIG. 6A, nanocomposites A, B, H, and G-Sr exhibited similar morphology, in which clusters of minerals formed “grape-shaped” micro-domains with a size of 100 to 200 nm in diameter. In contrast, mineral solution G resulted in a thin, “plate-like” morphology and mineral solution G-F resulted in clusters of “needle-like” morphology. Energy-dispersive x-ray spectroscopy (EDAX) was used to examine the composition of the nanocomposites. All mineral solutions resulted in structures containing calcium and phosphate (FIG. 6A insets). For nanocomposites A, B, and H, magnesium was detected whereas it was absent in nanocomposite G, G-Sr and G-F. For nanocomposites G-Sr and G-F, strontium and fluoride, respectively, were detected indicating their incorporation into the mineral structures. Semi-quantitative analysis with EDAX revealed varying calcium-to-phosphate (Ca/P) ratios for each mineral formulation (Table 5). Among all the mineral solutions, formulation G, which lacks magnesium, resulted in the highest Ca/P ratio (1.83±0.35) in nanocomposites. Incorporation of both magnesium (formulations A and B) and strontium (formulation G-Sr), resulted in a decrease of the Ca/P ratio, indicating that calcium in nanocomposites can be replaced by magnesium and strontium, respectively. Incorporation of fluoride (G-F) led to a slight decrease of the Ca/P ratio to 1.68±0.34. Taken together, the composition of CaP nanocomposites, which can potentially affect their environment responsiveness, and thus gene transfer efficiency, can be easily tailored by adjusting the formulation of mineral solutions.

TABLE 5
Chemical composition of mineral nanocomposites formed
after 24 h mineralization as determined by semi-quantitative
analysis of EDX spectra.

| | Ca:P | Mg:P | Sr:P | F:P |
|---|---|---|---|---|
| A | 1.55 ± 0.13 | 0.48 ± 0.10 | | |
| B | 1.20 ± 0.32 | 0.28 ± 0.11 | | |
| G | 1.83 ± 0.35 | | | |
| H | 0.79 ± 0.13 | 0.45 ± 0.12 | | |
| G-Sr | 1.36 ± 0.38 | | 0.48 ± 0.12 | |
| G-F | 1.68 ± 0.34 | | | 1.17 ± 0.40 |

Biocompatibility of Surface-Induced DNA-Doped Nanocomposites.

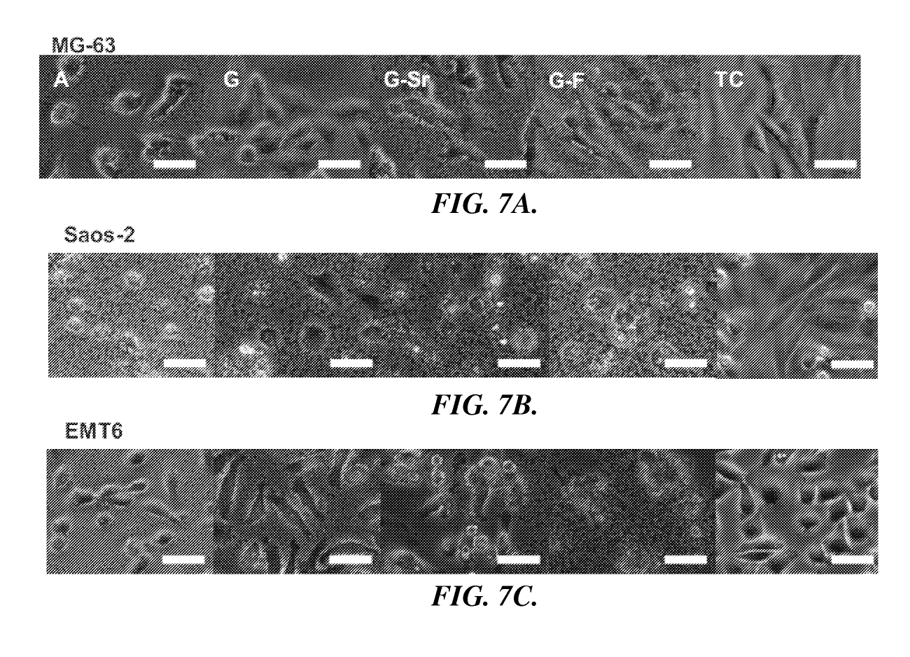

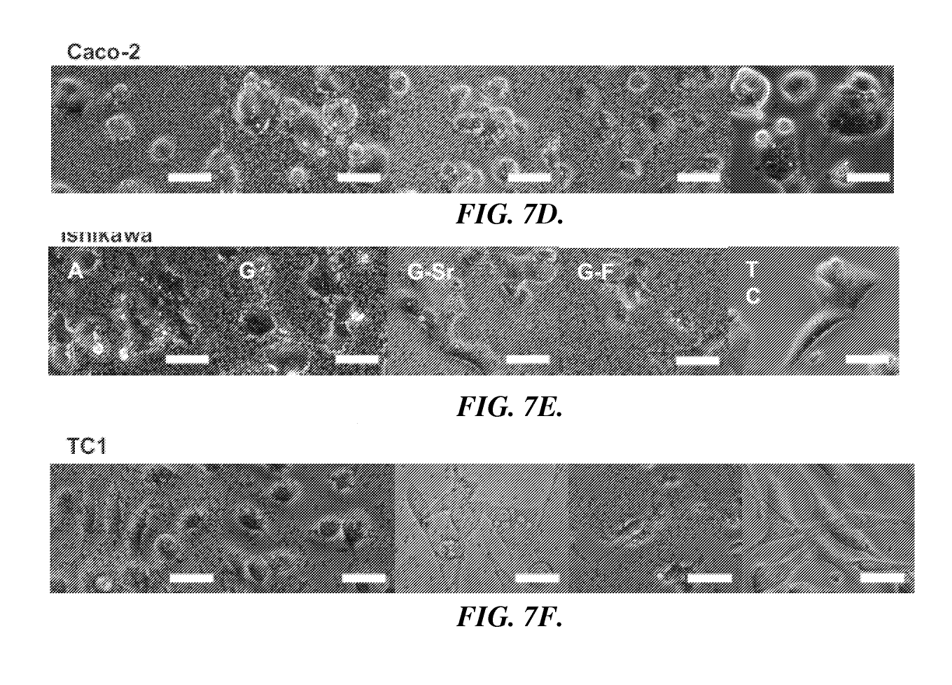

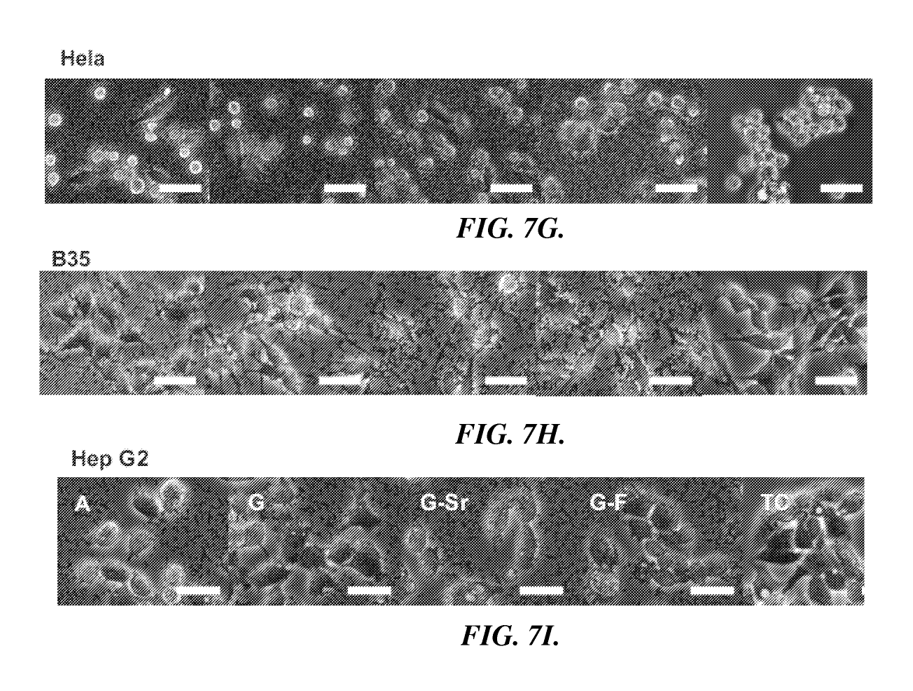

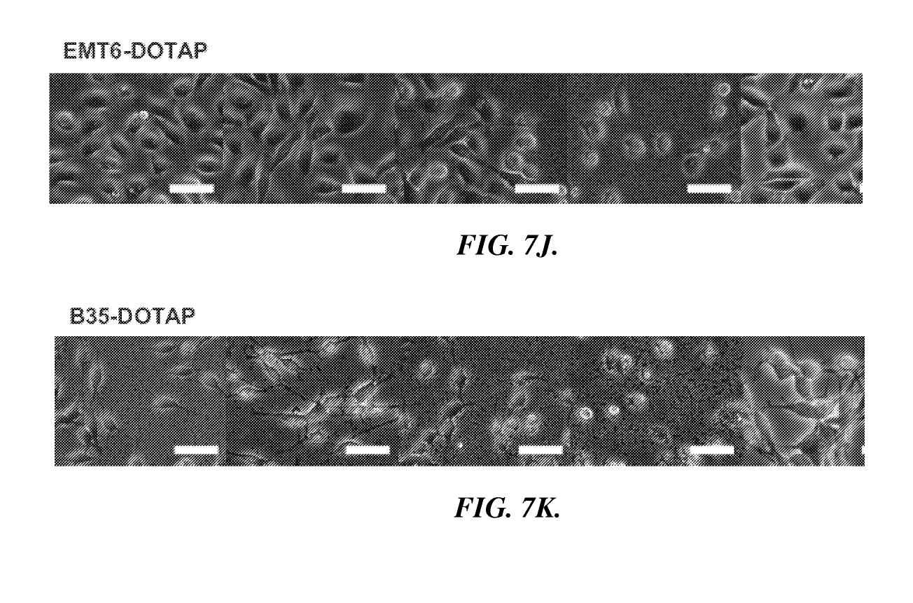

To establish a platform which could be used to screen and optimize the mineral compositions for any cells of interest, the biocompatibility of nanocomposites derived from the library in a variety of cell types was examined, including fibroblast, epithelial, and neuronal cells (Table 6). These cells are derived from various tissues, including bone, breast, colon, endometrium, lung, cervix, liver, and brain of different organisms. For all the cell types investigated, cells on the nanocomposite-coated surfaces displayed similar morphology as those on non-coated surfaces (FIG. 7). The majority of cell types covered similar surface areas on nanocomposite-coated surfaces as non-coated ones. Thus, nanocomposites did not adversely affect cell attachment and growth.

TABLE 6
Cell lines.

| | Cell type | Organ | Organism |
|---|---|---|---|
| MG-63 | fibroblast | bone | Human |
| Saos-2 | epithelial | bone | Human |
| EMT6 | epithelial | breast | Mouse |
| Caco-2 | epithelial | colon | Human |
| Ishikawa | epithelial | endometrium | Human |
| TC1 | epithelial | lung | Mouse |
| Hela | epithelial | cervix | Human |
| Hep G2 | epithelial | liver | Human |
| B35 | neuronal | nervous system | Rat |

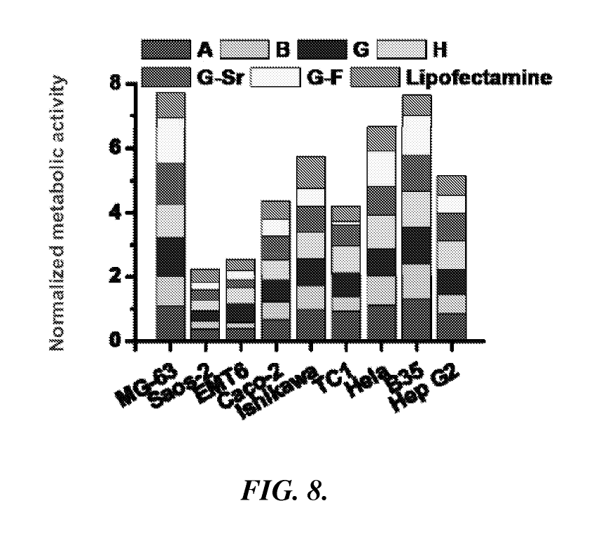

The cytotoxicity of nanocomposites was subsequently assessed by comparing the cellular metabolic activity of cells on nanocomposite-coated surfaces to that of cells on non-coated ones. DNA-doped nanocomposites did not induce significant levels of cell death and affect cell proliferation for the majority of cell types (FIG. 8). For MG-63, Caco-2, Ishikawa, TC1, Hela and Hep G2, cells proliferated normally on surfaces coated with nanocomposites derived from almost all the mineral formulations except G-F, on which there were 50% less viable cells than on non-coated surfaces for TC-1. For EMT6 cells, most mineral formulations significantly reduced the viability, except for G and H. For Saos-2, the relative metabolic activities of cells on all the six DNA-doped nanocomposites were only about 30% of that on non-coated surfaces. Previous studies have shown that the proliferation and differentiation of Saos-2, a human osteoblast-like cell line, was affected by mineral surfaces of different properties. Therefore, nanocomposite-coated surfaces can induce Saos-2 differentiation and reduce proliferation, which is consistent with previous observations.

Gene Transfer by DNA-Doped Nanocomposites in Nine Cell Types.

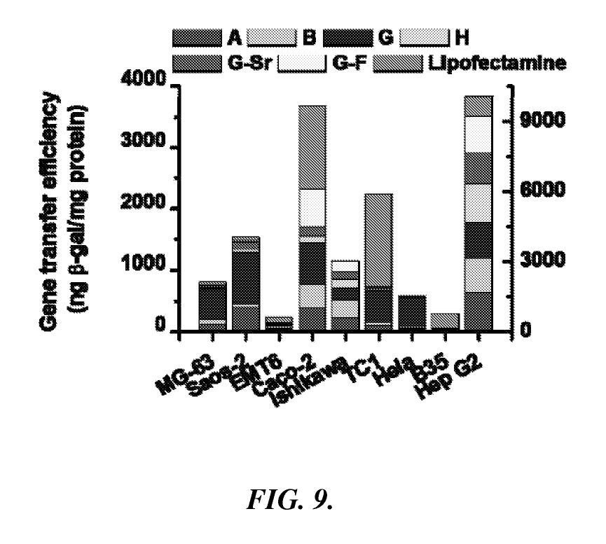

The ability of DNA-doped nanocomposites formed from the six mineral solutions to facilitate gene transfer to mammalian cells was tested on nine cell types (FIG. 9). DNA-doped nanocomposites derived from different mineral solutions yielded varying gene transfer efficiencies for all cell types. Interestingly, nanocomposite G, which lacks magnesium, resulted in the highest gene transfer efficiency in most of the cell types, with values ranging from 500 to 1500 ng β-gal/mg protein. In Ishikawa cells, nanocomposite B yielded the highest gene transfer efficiency, approximately 300 ng β-gal/mg protein. In contrast, nanocomposite H, which contains the highest level of magnesium, resulted in the lowest level of gene transfer efficiency in most cell types; nanocomposite G-Sr also yielded very low levels of gene transfer efficiency. In all examined cell lines but EMT6 and B35, there existed an optimized composition of nanocomposites that could efficiently deliver genes to cells. The gene transfer efficiency in most cell types by the optimized mineral formulation is comparable or even higher than that of a commercial reagent, Lipofectamine 2000™.

Correlation of Gene Transfer Efficiency with the Effectiveness of DNA Uptake.

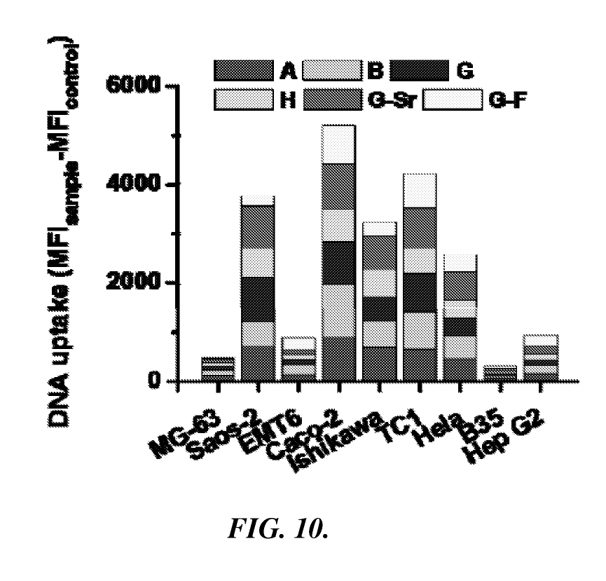

The nanocomposites formed from the initial library of mineral solutions and mineralization conditions achieved successful gene transfer in most of the cell types investigated except EMT6 and B35. Nanocomposites from different mineral solutions exhibit significant differences in the efficiency of gene transfer to a designated cell type. Cellular uptake and intracellular transport of DNA are two key steps for effective gene transfer. Initially, the gene transfer efficiency mediated by surface-induced nanocomposites was correlated with the level of cellular uptake. The uptake of DNA by cells seeded on the surfaces coated with nanocomposites was determined using flow cytometry (FIG. 10). DNA was effectively delivered into Saos-2, Caco-2, Ishikawa, TC1, and Hela by nanocomposites derived from almost all the mineral formulations except those from formulation G-F. Correspondingly, high levels of gene transfer efficiency were observed in those cells, particularly when nanocomposites from formulation G was used. DNA was poorly transported into MG-63, EMT6, B35 and Hep G2 by nanocomposites formed from the library of mineral formulations. For EMT6 and B35, the low gene transfer efficiency correlated with the low level of DNA uptake. Surprisingly, for MG-63 and Hep G2, though a much lower level of DNA was delivered into the cells compared to other cell types, the gene transfer efficiency was high, particularly in nanocomposite G.

Correlation of Gene Transfer Efficiency with the Deviation of pH Responsiveness of Nanocomposites from Early Phagosomal pH of Cells.

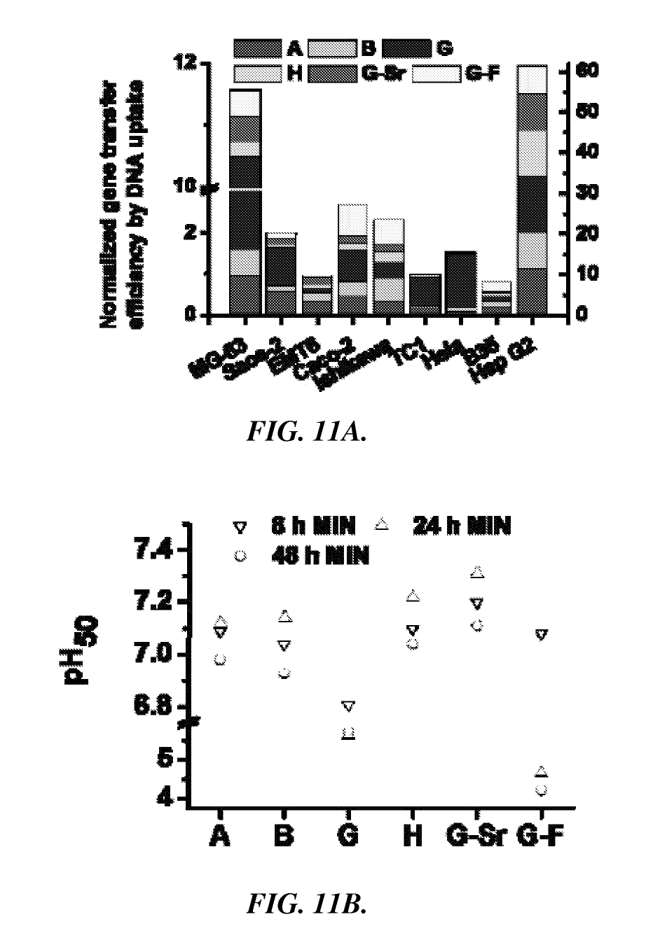

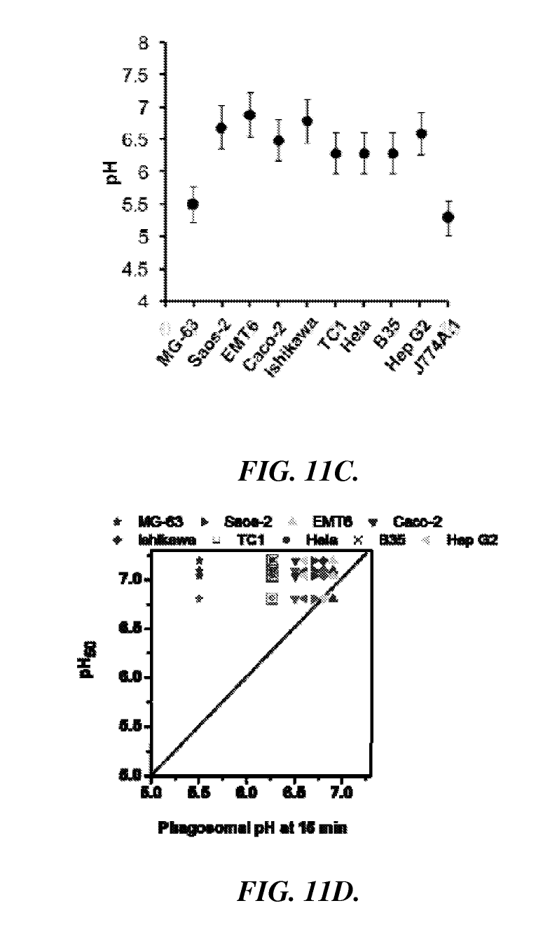

The efficiency of gene transfer in general correlates with the intracellular level of DNA across cell types. However, for each individual cell type, there was not a direct correlation between the level of intracellular DNA and the gene transfer efficiency of nanocomposites derived from different mineral formulations. After normalizing the gene transfer efficiency based on the amount of intracellular DNA to exclude the effect of uptake of DNA (FIG. 11A), in a designated cell type, nanocomposites derived from different formulations exhibited varying gene transfer efficiency per endocytosed DNA; for most of cell types, nanocomposite G demonstrated the highest gene transfer efficiency. Apparently, the efficiency of gene transfer by endocytosed DNA correlates both properties of nanocomposites and that of cells. The intracellular fate of DNA is closely associated with phagosomal pH and pH responsiveness of delivery systems. Therefore, it was investigated whether there is a correlation between the efficiency of gene transfer by endocytosed DNA, phagosomal pH of cells and pH responsiveness of nanocomposites.

pH responsiveness of DNA-doped nanocomposites was first examined. The pH at which 50% of the total calcium was released from nanocomposites, denoted as pH₅₀, was used as an indicator of pH responsiveness. As illustrated in FIG. 11B, nanocomposite A, B, H, and G-Sr, had pH₅₀ values ranging from 7.0-7.3 for the duration of mineralization examined. In contrast, nanocomposite G and G-F were more resistant to acidification, particularly as the mineralization duration was prolonged. For nanocomposite G, as the duration of mineralization increased from 8 h to 48 h, the pH₅₀ decreased from 6.8 to 5.6, respectively; for formulation G-F, the pH₅₀ decreased from 7.1 to 4.7 and finally to 4.2 as the mineralization time increased from 8 h, 24 h, to 48 h, respectively. Nanocomposites derived from the library of mineral solutions demonstrate appreciably different pH responsiveness.

The phagosomal pH of cells at 15 min post exposure to 100 nm-polystyrene beads was subsequently examined by a flow cytometry-based method. Different cell types exhibited significantly different phagosomal pH.

The normalized gene delivery efficiency was correlated with the deviation of pH₅₀ of nanocomposites from phagosomal pH of cells (FIG. 11D). For eight out nine cell types, nanocomposites with a pH₅₀ that displayed the slightest deviation from the early phagosomal pH of cells yielded the highest gene transfer efficiency per endocytosed DNA.

The results indicate that gene transfer efficiency medicated by DNA-doped nanocomposites strongly correlates with the cellular uptake of DNA and the deviation of pH₅₀ of nanocomposites from phagosomal pH of individual cell types. This correlation will enable us to customize the gene delivery system with respect to each individual cell type.

Optimization of Gene Transfer in B35 and EMT6 Cells.

Based on the preliminary analysis on the effect of cellular uptake and deviation of pH₅₀ of nanocomposites from phagosomal pH on gene transfer efficiency described above, improvement in gene transfer in B35 and EMT6 cells was sought. The cellular uptake in both cells was low compared to other cell types. The initial focus was on improving cellular uptake. It has been shown that the cellular uptake can be facilitated by high local levels of DNA. Therefore, the aim was to control the spatial distribution of DNA to achieve higher local concentrations of DNA molecules per cell.

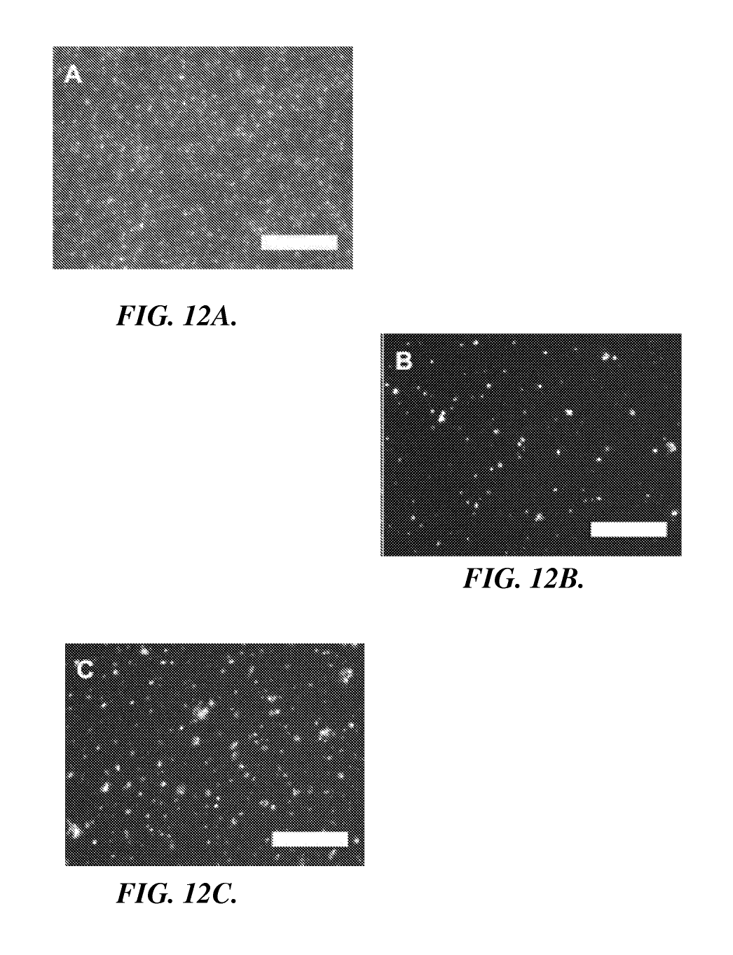

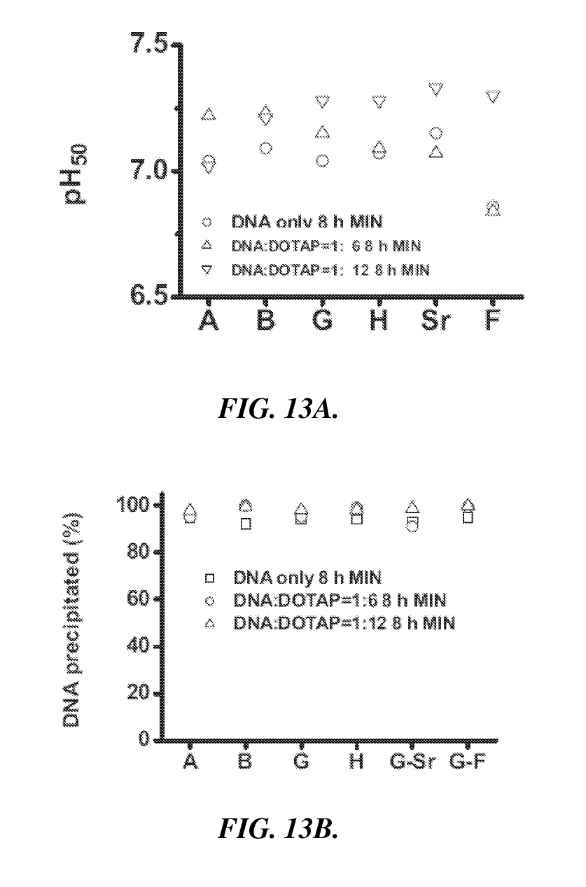

1,2-dioleoyl-3-trimethylammonium-propane (DOTAP), a cationic lipid able to condense DNA, has been widely used as a non-viral gene delivery vector. The DNA was initially complexed with DOTAP at two different ratios, followed by subsequent mineralization to form DNA/DOTAP nanocomposites. Fluorescent microscopy images of fluorescent DNA in modified nanocomposites revealed large, punctuate structures, suggesting a high local concentration of DNA (FIGS. 12A-12C); a higher DNA: DOTAP ratio (FIG. 12C) resulted in more localized distributions compared to a lower ratio (FIG. 12B). In contrast, DNA in un-modified nanocomposites showed a more diffuse distribution of DNA, suggesting lower local concentration of DNA (FIG. 12A). The inclusion of DOTAP slightly affected the pH responsiveness of nanocomposites, but not the DNA precipitation efficiency (FIGS. 13A and 13B).

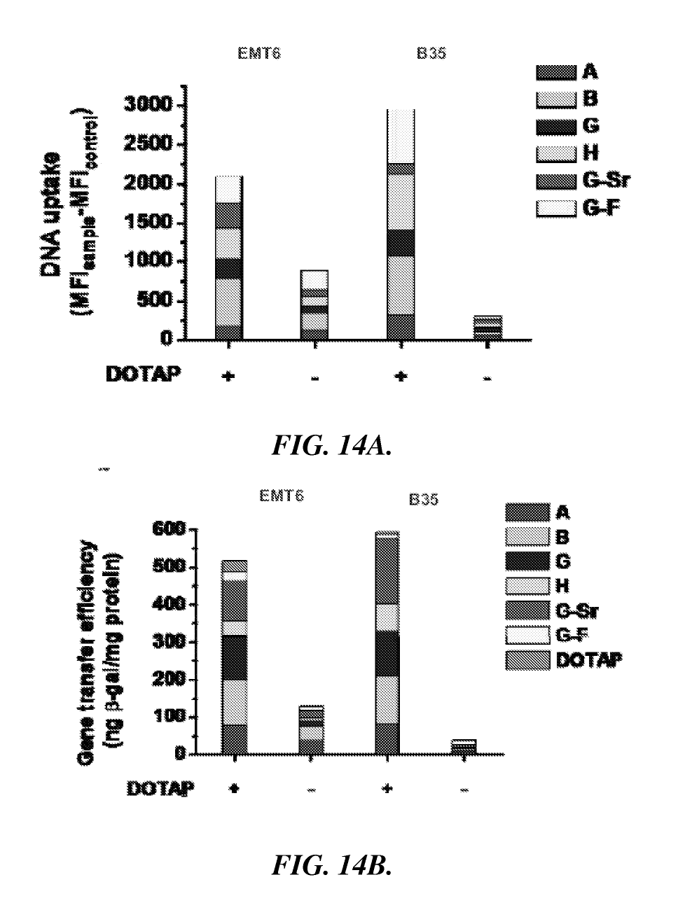

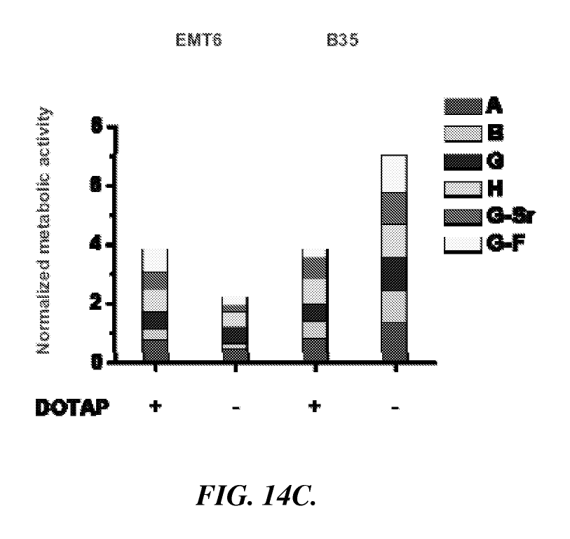

DOTAP-nanocomposites greatly enhanced the uptake of DNA compared to unmodified ones for both EMT-6 and B35 cell lines, respectively (FIG. 14A). Consequently, the gene transfer efficiency was markedly enhanced, with the greatest improvement shown by formulations B, G, and G-Sr (FIG. 14B). The gene transfer efficiency directly correlated with the enhanced uptake of DNA for all mineral formulations. Modified nanocomposites enhanced the viability of EMT6 cells, yet slightly reduced the viability of B35 cells compared to un-modified nanocomposites (FIG. 14C).

The gene transfer in nine cell types described above indicates that surface-induced nanocomposites provide a flexible platform to examine the efficiency of nanocomposites derived from a wide variety of mineral formulations in various cell types in a high throughput fashion.

Core-Shell Particles

The invention provides core-shell particles, compositions that include the particles, and methods for making and using the particles.

In one aspect of the invention, core-shell particles are provided. In one embodiment, the particle includes (a) a core comprising an inorganic mineral component; (b) a polymeric shell surrounding and substantially encapsulating the core; and (c) a therapeutic or diagnostic agent.

The particle core includes an inorganic mineral component can be prepared from calcium chloride, potassium dihydrogen phosphate, sodium chloride, potassium chloride, and sodium bicarbonate at concentrations sufficient to mimic the plasma concentrations of calcium, potassium, sodium, phosphate, and chloride ions (e.g., simulated body fluid). The inorganic mineral component therefore includes calcium, potassium, sodium, phosphate, and chloride ions. In certain embodiments, the inorganic mineral component includes one or more ions selected from magnesium ions (e.g., magnesium sulfate and/or magnesium chloride), strontium ions (e.g., strontium chloride), and fluoride ions (e.g., sodium fluoride).

The particle shell surrounding and substantially encapsulating the core is a polymeric shell (i.e., comprises one or more polymers). Suitable polymeric shells include biocompatible polymers. Representative biocompatible polymers include poly(lactide-co-glycolic acid) (PLGA) polymers, poly(lactic acid) (PLA) polymers, and poly(glycolic acid) (PGA) polymers. Other suitable biocompatible polymers include collagens, alginates, fibrins, elastins, chitosans, gelatins, hydroxyethyl celluloses, hydroxypropyl celluloses, and carboxymethyl celluloses. Further suitable biocompatible polymers include poly(vinyl alcohol)s, poly(ethylene glycol)s, pluronics, poly(vinylpyrrolidone)s, poly(ethylene terephthalate)s, poly(anhydride)s, and polypropylene fumarate)s.

In certain embodiments, the shell comprises one or more peptides, polypeptides, proteins, and fragments thereof. In certain embodiments, the shell comprises one or more lipids.

In certain embodiments, the core-shell particle has a shell having a thickness up to about 20 nm. In certain embodiments, the core-shell particle has a shell having a thickness of from about 5 to about 10 nm.

The core-shell particles include one or more therapeutic agents and/or diagnostic agents. In certain embodiments, the therapeutic agent is a biopolymer. Representative biopolymers include nucleic acids, polysaccharides, peptides, polypeptides, proteins, and fragments thereof. Representative nucleic acids include DNAs and RNAs. In certain embodiments, the therapeutic agent is a therapeutic small molecule. Representative therapeutic small molecules include chemotherapeutic agents and antimicrobial agents. Representative diagnostic agents include imaging agent such as magnetic resonance imaging agents and fluorescence imaging agents. The therapeutic or diagnostic agent can be dispersed throughout the core, dispersed throughout the shell, or dispersed throughout the core and the shell.

The core-shell particle can further include a targeting agent. Representative targeting agents include antibodies and fragments thereof, carbohydrates (e.g., glycans), aptamers, small molecules (e.g., toxins), polypeptides, and peptides.

In certain embodiments, the core-shell particle has a diameter from about 100 nm to about 10 μm. In certain other embodiments, the particle has a diameter from about 100 to about 500 nm.

In certain embodiments, the core-shell particle has a polydispersity index from about 0.1 to about 0.6. In certain other embodiments, the particle has a polydispersity index from about 0.1 to about 0.3.

In another aspect of the invention, a pharmaceutical composition useful for administration of a therapeutic or diagnostic agent is provided. The pharmaceutical composition includes a pharmaceutically acceptable carrier and a core-shell particle of the invention having a therapeutic or diagnostic agent dispersed therein.

In other aspects, the invention provides methods for using the core-shell particles of the invention.

In one embodiment, the invention provides a method for delivering a therapeutic or diagnostic agent to cell. In the method, a cell is contacted with a core-shell particle of the invention having a therapeutic agent dispersed therein.

In another embodiment, the invention provides a method for regulating the expression of a gene or gene product. In the method, a cell is contacted with a core-shell particle of the invention having dispersed therein a DNA or RNA effective to regulate the expression of the gene or gene product.

In a further embodiment, the invention provides a method for tissue engineering. In the method, cells of interest (e.g., stem cells) are mixed with a core-shell particle of the invention having a nucleic acid or a protein dispersed therein.

Microenvironment Regulation

The biomineral-containing composites of the invention are effective in regulating microenvironment. Thus, in another aspect, the invention provides a method for regulating an environmental parameter of an internal compartment of a cell. In the method, a cell is contacted with a colloidal particle of the invention or a core-shell particle of the invention. The colloidal particle or the core-shell particle is conducted to an internal compartment of a cell (e.g., by an endocytosis pathway) where the colloidal particle or core-shell particle is solubilized and changes the parameter to a predetermined value. Parameters in the microenvironment that can be affected include pH, ionic concentration, and osmotic pressure.

In another aspect of the invention, particle libraries are provided. The particle library includes of particle members, each particle member is effective to regulate a microenvironment parameter to a predetermined value. Each particle member comprises an inorganic mineral component comprising calcium ions, potassium ions, sodium ions, phosphate ions, and chloride ions, and the concentration of ions varies from member to member in a predetermined amount to provide the particle library. In certain embodiments, the inorganic mineral component further comprises one or more ions selected from strontium ions, magnesium ions, or fluoride ions. Parameters in the microenvironment that can be affected include pH, ionic concentration, and osmotic pressure. In one embodiment, the predetermined value is a pH value between 4.5 and 6.5.

As noted above, the invention provides nanocomposites having reduced immunogenicity, increased delivery efficiency, and tunable compositions and morphologies. The nanocomposites can be associated with an agent (e.g., therapeutic or diagnostic agent). Association with an agent contemplates either doping of the nanocomposite with the agent or use of the nanocomposite and agent in parallel. In addition, a plurality of agents can be used with one or more of them doped in the nanocomposite and fewer than all agents doped into the nanocomposite.

The nanocomposites can be used for the efficient delivery into cells of any suitable agent (e.g., DNA, siRNA, RNA-based agents, chemotherapeutic drugs, antibiotics, toll-like receptor agonists, vaccines). The nanocomposites affect a wide range of biological responses for a wide and varied range of cells. Also contemplated by the present disclosure is the deposition of the nanocomposites into tissue scaffolds (i.e., nanofibers) to deliver agents to cells for the application of tissue engineering.

In various aspects, the present disclosure provides methods for regulating a cellular process, comprising the steps of producing a nanocomposite composition comprising a nanoparticle and an agent and delivering the nanocomposite composition to a cell or tissue.

In various aspects, the present disclosure provides nanocomposite compositions comprising a nanoparticle and an agent, wherein the nanocomposite has a tunable pH responsiveness.

In various aspects, the nanoparticles comprise a plurality of materials. In certain aspects, the plurality comprises a core made of one material and at least one layer (i.e., shell) surrounding the core, wherein the layer comprises a material that is not the same as the core. In certain aspects, the shell comprises a polymer, protein, lipid, or combinations thereof, and the core comprises a biomineral. In certain aspects, the biomaterial has a composition selected from any one of the those describes herein. In certain aspects, the biomineral composition is selected according to the properties of the cell or tissue.

In certain aspects, the biomineral composition is prepared from a solution comprising about 0 mM to about 10 mM CaCl₂.2H₂O, about 0 mM to about 2 mM KH₂PO₄, about 100 mM to about 200 mM NaCl, about 2 mM to about 6 mM KCl, about 0 mM to about 20 mM MgSO₄.6H₂O, about 0 mM to about 10 mM MgCl₂.6 H₂O, about 3 mM to about 5 mM NaHCO₃, about 0 mM to about 10 mM SrCl₂, and about 0 mM to about 10 mM NaF.

In further aspects, the biomineral composition is prepared from a solution comprising about 0 mM to about 10 mM CaCl₂.2H₂O, about 1 mM KH₂PO₄, about 140 mM NaCl, about 4 mM KCl, about 0 mM to about 10 mM MgSO₄.6H₂O, about 0 mM to about 10 mM MgCl₂.6 H₂O, about 4 mM NaHCO₃, about 0 mM to about 10 mM SrCl₂, and about 0 mM to about 10 mM NaF.

In certain aspects, the nanocomposite composition is selected such that 50% of the nanocomposites dissolve at a pH of interest. In further aspects, the pH responsiveness is tuned to pH 4.0, 4.5, 5.0, 5.5, 6.0, 6.5, 7.0, 7.5, or 8.0. In some aspects, the pH responsiveness is tuned to tuned to pH 5.0, 5.5, or 6.0. In further aspects, the pH of the cellular environment is altered to pH 5.0, 5.5, or 6.0 by the nanocomposites.

In various aspects, the nanocomposite includes one or more targeting agents that enhances delivery of the agent. In certain aspects, the enhancer is enhancer is comprised of an antibody, glycan, polymer, or liposome.

In various aspects, the nanocomposite compositions regulate a cellular process is at least one of tissue generation, protein synthesis, gene expression, cell proliferation, mitosis, DNA transcription, hormone production, enzyme production, cytokine production, microbe reduction, cell death, innate immune responses, adaptive immune responses, gene delivery, vaccination, or drug delivery.

In various aspects, the agent is an oligonucleotide, DNA, siRNA, an RNA-based agent, a protein, a lipid, a carbohydrate, a small organic molecule drug, an antibiotic, a toll-like receptor agonist, an antibody, imaging agent, or a vaccine. In further aspects, the imaging agent is a quantum dot or contrast agent.

In certain aspects, the tissue is a living tissue, artificial tissue, or a tissue scaffold comprising polymers and inorganic material.

The present material systems are developed at the nano- and micro-scales for regulating at least one cellular function. Cellular process includes any process a cell undergoes or participates in, including bone formation, protein synthesis, cell repair, cell division, cell proliferation/mitosis, cell differentiation, cell death, gene expression, cell respiration, DNA transcription and drug delivery. The term “regulating” means altering at least one of quantity, speed, rate, efficiency, quality, or target delivery. Thus, for example, a material system can increase bone formation, alter apoptosis, or improve drug delivery. Regulating can also mean increase or decrease of a specific cellular function.

A material delivery system can comprise, or form from, any material suitable for delivery into a living organism. For example, a material system can be polymeric, biomineral, metal, metal oxide, ceramic, carbon-composite, cobalt-chromium, titanium alloy, and combinations thereof. A structured surface can be defined by, or composed of, or formed of a material that includes a plurality of particles that are sintered together to form a continuous porous phase.

A material can have any shape suitable for an intended purpose, as the circumstances present. One of ordinary skill in the art will understand the need for a particular material and will appreciate the shape or morphology necessary to accomplish a particular need. Material shapes include spheres (filled and unfilled), squares, cylinders, cubes, pods, cones, pyramids, and filaments. The structures can be at the nano, micro, or macro level and can have a plurality of shapes and dimensions. It is envisioned that the shapes could be spherical, tubular, cylindrical, triangular, plates, hexagonal, fibrous, or any morphological shape that can interact with cells in the desired manner. Also, it is possible to have a combination of structures with various shapes and structures in such a way that together or individually the system plays the desired role.

A material should be biocompatible or non-toxic with a living organism receiving said material. Biocompatibility can be accomplished by constructing a material system from material that will not interfere with a host organism's basic functions and/or coating a material's surface.

The present invention provides a material system for regulating at least one cellular process and comprises nanoparticles. The term “nanoparticle” refers to an object having a at least one nanometer-scale dimension (less than 1 micron) that behaves as a whole unit in terms of its transport and properties. Generally, nanoparticles have a size of about 0.5 nm to about 100 nm. In various aspects, the nanoparticles of the present disclosure are nanocomposites comprising a core and shell. In various aspects, the shell comprises a polymeric material.

In various aspects, the nanocomposites of the present disclosure comprise a core and polymer shell.

In certain aspects, the present disclosure provides methods of producing a nanocomposite composition, wherein the nanocomposite composition comprises a nanoparticle and an agent, comprising the steps of determining an optimum pH for delivery to a target cell type; determining the biomineral and polymer composition capable of producing the optimum pH; producing a nanocomposite composition, wherein the nanocomposite composition is capable of producing a target pH that approaches the optimum pH. In further aspects, the target pH is adjusted to favorably control endosomal pH. In certain aspects, the nanoparticle is doped with agent is an oligonucleotide, DNA, siRNA, an RNA-based agent, a protein, a lipid, a carbohydrate, a small organic molecule drug, an antibiotic, a toll-like receptor agonist, an antibody, imaging agent, or a vaccine.

In certain aspects, the nanocomposite composition is produced on a two-dimensional surface. In other aspects, the nanocomposite composition is produced on a three-dimensional surface. In further aspects, the surface is a living tissue, artificial tissue, or a tissue scaffold comprising polymers and inorganic material.

In further aspects, the nanocomposite composition is produced as a colloidal system.

The properties of the nanoparticles and the nature of the microenvironment created by the nanoparticles can be tuned by modifying the nanoparticle composition and process parameters.

Biocomposites according to the present disclosure can be applied to any tissue type.

Separately or in conjunction with the inventive material systems, the invention is effective in regulating a cellular process with a composition of nanocomposites. A nanocomposite composition comprises at least one nanoparticle associated with at least one agent. Exemplary agents include but are not limited to proteins, growth factors, hormones, antibodies, amino acids, carbohydrates, polymers, drugs, nucleic acids, enzymes, and the like. The agents can be associated with a nanoparticle by any suitable means. In various aspects, the nanoparticles are doped with agents. The systems can be delivered by injection, epidermal translation, inhalation, direct surgical placement, or any other suitable method known in the art.

In certain aspects, such as the use of the present invention for the generation of tissue scaffolds, the nanoparticles are doped with a stimulating agent. A stimulating agent can be at least one protein, growth factor, antibody, amino acid, polymer, drug, nucleic acid, hormone, and/or enzyme. For example, a stimulating agent can be a bone morphogenic protein (BMP), which can stimulate mesenchymal/stem cells to differentiate into an osteoblast cell.

As used herein, a growth factor is a naturally occurring substance capable of stimulating cellular growth, proliferation, and/or cellular differentiation. Generally, a growth factor is a protein or a steroid hormone, and typically acts as a signaling messenger between cells. Relevant to an enhanced cellular function, a growth factor can promote cell differentiation, cell growth, protein synthesis, and/or gene expression, each of which varies based on the particular growth factor employed. For example, bone morphogenic proteins (BMPs) stimulate bone cell differentiation, while fibroblast growth factors and vascular endothelial growth factors (VEGF) stimulate blood vessel differentiation (angiogenesis).

Non-limiting exemplary growth factors include Bone Morphogenic Proteins (BMPs), Brain-Derived Neutrophic Factor (BDNF), Ciliary Neutrophic Factor (CNTF), Epidermal Growth Factor (EGF), Erythropoietin (EPO), Fibroblast Growth Factor (FGF), Granulocyte-Colony Stimulating Factor (G-CSF), Granulocyte-Macrophage Colony Stimulating Factor (GM-CSF), Growth Differentiation Factor-9 (GDF9), Hepatocyte Growth Factor (HGF), Insulin-like Growth Factor (IGF), Interleukin (IL), Leukemia Inhibitory Factor (LIF), Myostatin (GDF-8), Nerve Growth Factor (NGF), Neutrophic Factors (NT), Platelet-derived Growth Factor (PDGF), Thrombopoietin (TPO), Transforming Growth Factor alpha(TGF-α), Transforming Growth Factor beta (TGF-β), and Vascular Endothelial Growth Factor (VEGF).

As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins can be derived from natural sources, or partly or wholly synthetically produced. An antibody can be monoclonal or polyclonal. An antibody can be a member of any immunoglobulin class, including any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include but are not limited to Fab, Fab′, F(ab′)2, scFv, Fv, dsFv diabody, and Fd fragments. An antibody fragment can be produced by any means. For example, an antibody fragment can be enzymatically or chemically produced by fragmentation of an intact antibody and/or it can be recombinantly produced from a gene encoding the partial antibody sequence. Alternatively or additionally, an antibody fragment can be wholly or partially synthetically produced. An antibody fragment can optionally comprise a single chain antibody fragment. Alternatively or additionally, an antibody fragment can comprise multiple chains which are linked together, for example, by disulfide linkages. An antibody fragment can optionally comprise a multimolecular complex. A functional antibody fragment typically comprises at least about 50 amino acids and more typically comprises at least about 200 amino acids.

Any drug or therapeutic agent can be used in a nanocomposite composition. In various aspects, the agent is a clinically-used drug including but not limited to an antibiotic, antifungal agent, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, anti-hypertensive, sedative, progestational agent, anti-cholinergic, analgesic, anti-depressant, anti-psychotic, β-adrenergic blocking agent, diuretic, cardiovascular active agent, vasoactive agent, and non-steroidal anti-inflammatory agent. A drug or therapeutic agent can be a mixture of pharmaceutically active agents. For example, a local anesthetic can be delivered in combination with an anti-inflammatory agent such as a steroid. Local anesthetics can also be administered with vasoactive agents such as epinephrine.

In certain aspects, the nanocomposite composition comprises DNA and is used for gene therapy.

The nanocomposites can be delivered by any suitable method known in the art. For example, and in no way limiting, a material system can be delivered by direct injection, epidermal translation, inhalation, direct surgical placement, or the like. Delivery can be directed to any cell type or tissue.

In various aspects, the nanocomposites can be delivered to any eukaryotic cell or tissue of interest. In certain aspects, a cell is a mammalian cell. Cells can be of human or non-human origin. For example, they can be of mouse, rat, or non-human primate origin. Exemplary cell types include but are not limited to endothelial cells, epithelial cells, mesenchymal cells, stem cells, muscle cells, neurons, hepatocytes, myocytes, chondrocytes, osteoblasts, osteoclasts, lymphocytes, macrophages, neutrophils, fibroblasts, and keratinocytes. Cells can be primary cells, immortalized cells, transformed cells, terminally differentiated cells, stem cells (e.g., adult or embryonic stem cells, hematopoietic stem cells), somatic cells, germ cells, and the like. Cells can be wild type or mutant cells, e.g., they can have a mutation in one or more genes. Cells can be quiescent or actively proliferating. Cells can be in any stage of the cell cycle. In some embodiments, cells can be in the context of a tissue. In some embodiments, cells can be in the context of an organism.

Cells can be normal cells or diseased cells. In certain embodiments, cells are cancer cells, e.g., they originate from a tumor or have been transformed in cell culture (e.g., by transfection with an oncogene). In certain embodiments, cells are infected with a virus or other infectious agent. A virus can be, e.g., a DNA virus, RNA virus, retrovirus, or the like. For example, cells can be infected with a human pathogen such as a hepatitis virus, a respiratory virus, human immunodeficiency virus, or the like.

Cells can be cells of a cell line. Exemplary cell lines include MG-63, Saos-2, EMT6, Caco-2, Ishikawa, HeLa, TC1, B35, Hep G2, CHO, COS, BHK, NIH-3T3, and HUVEC. For an extensive list of cell lines, one of ordinary skill in the art can refer to the American Type Culture Collection catalog (ATCC™, Manassas, Va.).

In some aspects, speed or delivery rate to a cell type and/or tissue can be increased by exposing said cell and/or tissue comprising a material system to radiation, which permits faster penetration of the host cell and/or tissue. Any suitable radiation technique can be used, including laser radiation and electromagnetic radiation.

In certain aspects, the present disclosure provides for microenvironment pH controllers. In certain embodiments, pH controllers comprise a biomineral core and polymer shell, resulting from biomineralization within a nano/submicron polymeric sphere. Biominerals are mineral deposits of the type that are produced by the action of an organism. In light of the fact that biomineral dissolution is sensitive to and can regulate its local environmental change (i.e., pH), biominerals are ideal candidates for pH controllers. Both biomineral core and polymer shell can be precisely tuned to possess differential responses to pH. Nano/submicron dimensions allow the controllers to transport across cellular and tissue barriers. The tunable surface chemistry of polymer shell can direct controllers to cells or tissues of interest.

In certain specific aspects, controllers can be designed to regulate the endocytosis pathway, which is a vital process for maintaining both cellular and organismal functions. An application of these controllers is to guide toll-like receptor-mediated innate immunity through regulating the endocytosis pathway.

The ability to monitor and control microchemical environments in sub-cellular compartments is critical for understanding life, diagnosing and curing diseases. In certain aspects, microenvironment pH controllers composed of a mineral core-polymer shell are provided, which result from biomineralization within a sub-micron polymeric sphere. In various aspects, those spheres are comprised of a biomineral core and polymer shell, both of which can be precisely tuned to possess differential responses to pH. Sub-micron dimensions enable the controllers to transport across cellular and tissue barriers. The tunable surface chemistry of polymer shell can direct controllers to cells or tissues of interest. In certain aspects, controllers regulate the endocytosis pathway, which is a vital process for maintaining both cellular and organismal functions. In certain aspects, the present controllers are used to guide toll-like receptor (TLR)-mediated innate immunity through regulating the endocytosis pathway.

The endocytosis pathways exist in all eukaryotic cells. At a minimum, endocytosis maintains cellular homeostasis by recovering protein and lipid components inserted into the cell membrane by ongoing secretory activity. Endocytosis is also critical for organismal homeostasis, controlling an extraordinary array of activities that every cell must exhibit in order to exist as part of a multicellular community. These activities include the transmission of neuronal, metabolic, and proliferative signals; the acquisition of nutrients; regulated communication with the external world; and the establishment of effective innate or adaptive defense mechanism against invading microorganisms. This pathway has also been explored as a means for delivering therapeutic agents across cell membranes. Paradoxically, many infectious agents take advantage of this pathway to effectively invade the intended host. Given the importance of this pathway, the ability to control and monitor this pathway provides a significant tool for improving human health.

Endocytosis is characterized by the continuous and regulated formation of prolific membrane vesicles, endosomes. Endosomes are not static but dynamic in both their chemical contents and spatial locations in a cell, which depends on intra-endosomal pH. In certain aspects, microenvironment pH controllers, composed of biomineral core and polymer shell, are used to control the pH change in endosomes, and thus to control the dynamics of endosomes and their cargos. pH controllers act by guiding TLR-based innate immunity for maximum effectiveness and minimal side effects.

Innate immunity provides the first line of defense against pathogens by delaying their replication and shaping the adaptive immunity. This is accomplished through TLRs and other pattern recognition receptors. The TLR family comprises of 12 members. Based on their subcellular locations, the TLR family can be roughly categorized into cell-surface or endosomic receptors. Cell-surface receptors recognize bacterial and protozoan pathogens, and include TLRs 1, 2, 4, 5, 6, and 11; endosomic receptors, situated in endosomes, recognize viruses within the acidified endosomes, and include TLRs 3, 7, 8 and 9.

The activation of TLRs 3, 7 and 9 can induce the production of both type I interferons (IFNs) and proinflammatory cytokines (IL-6, IL-12 and TNFα). Type I IFNs are effector cytokines which control viral infections while proinflammatory cytokines induce undesired inflammatory responses. Some studies suggest that uncontrolled production of type I IFNs can lead to autoimmune diseases. The induction of two types of cytokines is regulated primarily at the transcriptional level, where IFN regulatory factors (IRFs) play central roles. For TLRs 7/9, two IRFs, IRF-5 and IRF-7, have been shown to have distinct roles in inducing inflammatory cytokines and type I IFNs. IRF-5 regulates the production of pro-inflammatory cytokines while IRF-7 regulates the induction of type I IFNs 25-26. Interestingly, while IRF-4 is recruited, the cytokine production is turned off. In contrast, activation of TLR3 activates IRF-3 through Toll/IL-1 receptor domain-containing adapter-inducing IFN-β (TRIF), leading mostly to IFNβ secretion. The recruitment and activation of IRF-3, 5 and 7 depends on the nature of ligands and cell types. A recent study, however, has suggested the difference of type I IFNs production observed in different cell types or induced by different ligands is due to the difference in the dynamics of intracellular trafficking of TLR ligands, and thus differential recruitment of IRF-5 or -7. Prolonged retention of TLR ligands in endosomes rather than lysosomes induced higher levels of type I IFNs.

The present disclosure relates in part to the discovery that cytokine production (type I IFNs vs. proinflammatory cytokines) can be modulated by controlling the dynamics of endosomes, and thus the intracellular distribution of TLR-stimulating agents, through pH controllers. Advantageously, immunogenicity can be dramatically reduced through the use of the present compositions and methods.

Endocytosis is an essential process for homeostasis of both cells and organs. Endocytosis results in the formation of endosomes, intracellular structures that shuttle cargos to various locations for different purposes. The dynamics of endosomes refers to the process by which early endosomes (pH=6.0) progress into late endosomes (pH=5.0 to 6.0), and fuse with lysosomes (pH=4.5 to 5.0). This process is directly coupled with the acidification of endosomes, which is accomplished through vacuolar (H⁺)-ATPase (V-ATPase). Therefore, the regulation of pH can potentially tune the dynamics of endosomes. In certain aspects, pH controllers are provided comprising a nano/submicron polymeric shell and biomineral core. In further aspects, biominerals are used as pH controllers because their dissolution is sensitive to and can regulate pH changes. The polymer shell can comprise any suitable polymeric material, e.g., poly(lactide-co-glycolide acid) (PLGA) polymer or other biocompatible polymers. pH microcontrollers offer many advantages: first, both polymer and biominerals are tunable so that we can finely control the dynamics of endosomes; second, size and surface chemistry of polymeric particles can also be easily controlled to target desired cell populations; third, unlike many other pH-responsive polymers or lipids, biominerals can be tuned to introduce minimal disturbance to the cellular environment.

In certain aspects, the pH controller comprises a mineral core within a nano/submicron polymer shell through biomineralization. Due to nano/submicron dimensions, the pH controllers can be readily ingested by cells through endocytosis. Various cell types have differing ability to ingest particles, so the size of pH controllers can be tuned to facilitate endocytosis for those varying cell types. The initial acidification of endosomes leads to the dissolution of minerals, which will consume protons and compensate for pH decrease due to protons pumped in by V-ATPase. As a result, the pH can be stabilized at desired pHs for desired durations depending on the dissolution kinetics and total amount of minerals incorporated in the controllers.

In some aspects, the pH controllers are produced such that they give rise to a particular pH. The pH produced by the pH controller can be any pH suitable for use with the delivery of agents of the present disclosure and with endosomes as presently described. In certain aspects, the pH is 4.0, 4.5, 5.0, 5.5, 6.0, 6.5, 7.0, 7.5, or 8.0. In certain specific aspects, a series of three pH-controllers are used having pHs of 6.0, 5.5 and 5.0, which corresponds to the pH at early endosomes (EE), late endsomes (LE) and lysosomes (LY), respectively. In certain aspects, controllers are designed to maintain the desired pH for three durations.

Without being bound by any particular theory, endosomal pH is in general is thought to be controlled by two processes: (1) protons are pumped into endosomes by vacuolar (H⁺)-ATPase (V-ATPase) and (2) protons are passively leaked out of endosomes. Initially, the second process is much slower than the first process, and thus, the endosomes are acidified until proton leakage balances the first process, resulting in a steady-state. Some studies have also suggested that protons are consumed by the products of NADPK oxidase. The pH controllers of the present disclosure can be designed to adapt to this and other variations.

The mass balance of protons in endosomes is shown in Equation 1 below. At a given desired pH, the proton flux due to V-ATPase pumping and passive diffusion can be treated as constant, which fixes R_{pHdesired} and can be estimated through the equation. The parameters used in the equation can be estimated.

$${V}_{endosome} \frac{d [ {H}^{+} ]}{d t} = {J}_{V \text{-} ATPase} - {J}_{diffusion} - {V}_{controller} R \\ {V}_{V \text{-} ATPase} = {{nk}_{V \text{-} ATPase} ( Δ [ {H}^{+} ] )}^{- {m}_{V \text{-} ATPase}} \\ {J}_{diffusion} = {A}_{endosome} {k}_{diffusion} Δ [ {H}^{+} ] \\ R = {{k}_{controller} [ {H}^{+} ]}^{{m}_{controller}} \qquad Equation 1$$

wherein J is the flux of protons; V is volume; A is surface area; m is reaction order; Δ[H₊] is the difference of proton concentration across the endosomal membrane; R is proton consumption rate by the pH controller; n is the number of V-ATPase; k_{V-ATPase} is the rate constant determined by the chemical potential of ATP hydrolysis and the electrical and chemical potential difference of protons across the endosomal membrane; k_{diffusion} is the permeability of protons across the endosomal membrane; and k_{controller} is the effective rate constant of proton consumption by pH controllers.

Thus, the pH at pH_{desired} can be controlled, as long as the dependence of R (proton consumption rate) on pH, which depends on the proton diffusion across the polymer shell and the mineral dissolution rate on pH, intersects with the critical point. To achieve this, the composition of the mineral core and polymer shell is modulated, or the function of the V-ATPase by incorporating V-ATPase inhibitors in the pH-controller. The duration at a desired pH in endosomes is controlled by the amount of minerals incorporated per controller. This can be calculated based on the mineral dissolution kinetics.

The relative undersaturation of lattice ions also affects the mineral dissolution, which can cause the pH dependence of proton consumption to deviate from the critical point. The relative undersaturation, σ, can be expressed as:

$$σ = 1 - {( {\prod }_{n} {a}_{i} / {K}_{sp} )}^{1 / n} \qquad Equation ( 2 )$$

wherein Kₛₚ is the equilibrium constant, aᵢ is the activity of lattice ions in the solution, and n is the total number of lattice ion type. At a given pH, the dissolution of minerals is proportional to the relative undersaturation. By choosing minerals with very small Kₛₚ values or σ is near zero at the pH above the desired pH, then there is no dissolution. As pH decreases, a, of one ion species will be reduced. As a result, σ increases and the dissolution rate increases. Therefore, the dissolution of minerals is mainly dependent on pH changes.

The dissolution of biominerals is a poorly understood process, particularly in the microenvironment of biological systems. Continuous efforts have been devoted to studying this fascinating but complex process. Many minerals with varying dissolution pH and rate have been experimentally studied, and thus, mineral composition can be selected based on these criteria. The compositions can be adjusted to achieve proton consumption profiles at a given pH.

Mineral composition can be designed by using simulated body fluid (SBF) as a reference (Composition A in Table 4). By using SBF (A) and its variations (G, B, and H), minerals can be formed on polystyrene surfaces and PLGA particles. Both cationic and anionic ions of SBF can be replaced to achieve desired dissolution kinetics. In addition, adjusting properties of the polymer shell, such as blending ion-selective polymers into the shell, provides an extra level of tunability and therefore, choice of mineral compositions become less stringent.

Biomineralization involves two critical components: mineral solution and a template or matrix. For mineral nucleation and growth to occur, the chemistry and physical structure of templates must provide isolated niches from the environment. These isolated niches must be able to modify the activity of at least one mineral constituent (usually the cation) as well as protons and possibly other ions in order to maintain sufficient local supersaturation. Mineral solutions dictate the chemical composition, size, morphology and texture of formed minerals. Most biological systems use a physical delimiting geometry, e.g., intracellular vesicles or intercellular zones formed by several organisms, to create a niche and template to control the nucleation and growth of minerals. By mimicking biological systems, numerous synthetic templates are generated, including langmuir monolayers, self-assembled monolayers, microemulsions, vesicles, patterned surfaces, synthetic polymers, and biological molecules. In certain aspects of the present disclosure, PLGA or other polymeric particles will be used as templates.

Controlled supersaturation environment, size, and number of nucleation seeds are important for mineralization to occur. First, nucleation centers are incorporated into polymeric particles by a microemulsion method. Macromolecules, i.e., DNA, peptides, oligonucleotides and proteins, can function as both nucleation centers and/or mineralization templates. Macromolecules can be easily incorporated into polymeric particles through microemulsions. In certain aspects, the pre-formed mineral seeds or metal nanoparticles are co-incorporated into PLGA with TLR-stimulating agents. In other aspects, the PLGA polymer or other polymers are modified with compounds containing functional groups such as —COO⁻, —SO₃⁻, —PO₃H⁻, —OH⁻, which can facilitate biomineralization. In further aspects, polyelectrolyte polymers with the aforementioned functional groups are used to promote mineralization. They can be doped into PLGA or be used alone, depending on whether they can form particles by microemulsion.

A controlled supersaturation environment is then created inside polymeric particles by gradually extracting calcium ions from the external aqueous solution before the hardening of the polymeric shell. After mineralization occurs, the composition of mineral solution inside the polymer shell decreases, and the mineralization ceases. At this stage, dissolution of minerals can occur. Therefore, steps should be taken to maintain the mineral concentration inside the polymeric shell. Calcium and other lattice ions can be excluded from the polymeric network. To continuously maintain the concentration of these ions inside polymeric particles, metal chelating agents, which can increase the partition of calcium ions in the polymer phase, are incorporated. Hardening of polymeric particles can be controlled by selection of a solvent having suitable volatility.

The resulting pH controllers are characterized in several aspects: size and morphology; composition and dissolution kinetics of minerals. In certain aspects, the size and morphology of pH controllers can be characterized by scanning electron microscopy (SEM) and dynamic light scattering (DLS). In further aspects, the composition of minerals can be characterized by atomic absorption spectroscopy, Fourier transform infrared spectroscopy and/or colorimetric chemical assays.

Dissolution kinetics have been studied by various methods. In some aspects, a variation of the constant composition approach is used to characterize dissolution kinetics. Through some adaptation, this approach is applicable to the cellular environment. According to that method, the dissolution of minerals can be analyzed in serum-containing simulated body fluid (SBF) to mimic the cellular environment at a given pH at physiological temperature (37° C.). The dissolution of minerals leads to an increase in pH, however, the pH can be maintained at a desired level using a titrant made of HNO₃— and SBF. pH can be monitored by a pH probe, which can be coupled to a syringe pump. According to this method, a pH change triggers the syringe pump to add the titrant. The dissolution rate is then calculated from rate of titrant addition. The dissolution kinetics of this system are sufficiently similar to that of the cellular environment such that suitable mineral compositions can be identified using this system for subsequent testing in a cellular environment.

Reliable methods for in situ determination of the pH of endocytic organelles have been developed. Two pH-sensitive dyes, fluorescein isothiocyanate (FITC) and Oregon Green 514 are incorporated into the polymer shell of pH-controllers. By using these two dyes, pH changes between 4 and 7.5 can be tracked. The cells exposed to pH controllers can be imaged by fluorescent microscopy at given time points by rapidly alternating between 485 nm and 438 nm excitation filters while using a 505 nm dichroic and a 535 nm emission filter. The ratio of intensity at 505 nm to that at 535 nm proportionally changes as pH changes. Based on a calibration curve, the pH change versus time is obtained. The detailed method is described by Jankowski et al. (Jankowski et al., J Biol Chem, 277(8):6059-6066 (2002)).

The dissolution of minerals in endosomes can also be monitored. The dissolution of minerals results in the release of calcium or other metal ions into endosomes. Therefore, the rate of ion release from endosomes by metal ion-specific dyes (Invitrogen) can be monitored. For example, in certain aspects, Fluo-4, a dye that becomes fluorescent after binding to free calcium ions, is used to monitor the release of calcium in endosomes (Invitrogen). By incorporating Fluo-4 into the polymer shell, it is released into endosomes immediately after the uptake of pH controllers. By monitoring and quantifying the fluorescent intensity of Fluo-4 versus time in endosomes, the dissolution kinetics can be derived.

By incorporating the kinetics of pH change and the proton consumption kinetics derived from the dissolution kinetics of minerals into Equation 1, it can be determined whether there are other pH-regulating processes that are presently unknown. pH controllers can serve as a tool to elucidate basic mechanisms of the endocytosis pathway. Other pH controllers are contemplated by the present disclosure and can be identified using the methods described herein.

In various aspects, the present disclosure provides for microenvironment pH-controllers that can precisely regulate the pH change of endosomal compartments and thus, the dynamics of endosomes. The present intracellular pH controllers are useful for a variety of purposes, including e.g., modulating innate immunity, drug delivery, genetic material delivery and understanding signaling pathways involved in endosomal trafficking. Alternate strategies are presented herein for designing and fabricating pH controllers, and any of these methods can be used according to the present disclosure for the identification, production, and use of pH controllers. Besides the mechanisms discussed above, there can be additional mechanisms affecting endosomal pH.

The nanocomposites of the present disclosure can be produced using surface-mediated delivery of agents in order to maximize the agent delivery. Surface-mediated delivery can be facilitated through the use of any suitable means, including, without limitation. For example, various delivery systems are known, such as encapsulation in liposomes, microparticles, microcapsules, and capsules.

In certain aspects, the present nanocomposites are used for gene delivery. In further aspects, liposomes can be used to enhance surface-mediated delivery of genes. The level of transgene expression can be tuned by manipulating the composition of mineral solutions.

Immobilized naked DNA is uniformly embedded in thin films of biominerals, which limits the internalization of DNA to some cell types, such as neuronal cells. As provided in the present disclosure, DNA molecules can be complexed with liposomes to form lipoplexes. Subsequently, these lipoplexes can be immobilized onto a cell-culture compatible surface through surface-induced biomineralization. The lipoplexes modulated the architecture of biominerals while being immobilized onto the surface by the biominerals. Regardless of the formulations of mineral solutions, lipoplexes can be efficiently immobilized on the surface. For example, the intracellular level of DNA and transgene expression are greatly enhanced in neuronal cells compared to the immobilized naked DNA. Moreover, the level of transgene expression can be regulated by both the composition of nanocomposites and physicochemical properties of lipoplexes.

Surface-induced biomineralization represents a flexible and alternative approach to immobilize DNA onto substrates; the immobilization efficiency has been shown to be nearly 100%, regardless of the composition of the mineral solutions used. More importantly, the level of transgene expression can be easily tuned by manipulating the composition of mineral solutions with respect to individual cell types. However, the internalization of DNA is cell type-dependent. A low level of cellular DNA uptake mediated by surface-induced biomineralization has been observed in several cell types such as neuronal cells and breast cancer cells. When naked DNA is immobilized by surface-induced biomineralization, DNA is embedded in a continuous thin film of biominerals. This architecture results in strong binding between biominerals and biomaterial surfaces, which limits the effective release of DNA for internalization by cells. Therefore, controlling the architecture of DNA-doped biominerals, called DNA-nanocomposites, would improve the internalization of DNA by cells and thus enhance the level of transgene expression.

DNA and cationic liposomes are often rapidly self-assembled as supramolecular structures. Depending on the type of liposomes, the ratio of DNA to liposomes and the complexation duration, several structures, including multilamellar structure with alternating lipid bilayer and DNA monolayers, columnar inverted hexagonal structures, and spaghetti-meatball assemblies, have been observed. For all the structures, DNA is shielded by lipids. Lipid monolayers and vesicles have been used as templates for the induction of mineralization. These supramolecular structures can be used as templates to control the architecture of biominerals on the surfaces. A recent study has shown that lipoplexes formed from DNA and Lipofectamine™ 2000 can be incorporated into biominerals induced by a simulated body fluid. However, this study fails to provide clarity regarding the impact on the architecture of surface-induced nanocomposites with varying compositions from the fabrication process of liposomes, the size of liposomes, and the charge ratio of DNA to cationic lipids (and thus, the DNA distribution and cellular uptake and gene transfer efficiency).

According to the present disclosure, immobilization of lipoplexes by surface-induced biomineralization modulates the architecture of resulting nanocomposites on the surfaces of biomaterials and thus the internalization of DNA to cells. As a result, the cellular uptake of DNA and transgene expression are greatly enhanced in neuronal cells compared to immobilized naked DNA. The gene transfer efficiency can be tuned by the composition of mineral solutions, the initial size of the liposomes, the duration of complexation, and the ratio of liposomes to DNA (w/w). Surface-induced biomineralization represents a flexible approach to immobilize DNA in the form of either DNA or lipoplexes onto biomaterial surfaces. Various processes have been developed to immobilize naked DNA onto both 2-D and 3-D tissue scaffolds by surface-induced mineralization. In these studies, the process of mineralization was not altered but only involved replacing naked DNA with lipoplexes. Therefore, the presently described processes can be adapted for lipoplexes. The present disclosure demonstrates that surface-induced biomineralization holds great promise to enable surface-mediated DNA delivery to mediate functions of cells present in tissue engineering scaffolds.

In various aspects of the present disclosure, surface patterning can be used in order to tune the morphology and surface chemistry of the presently described nanocomposites.

Hybrid materials, which exhibit synergistic and complementary properties between two component materials, have demonstrated great potential in medicine, electronics and catalysis. The process of biomineralization, with the ability to direct the assembly of inorganic structures on a variety of organic templates, has spurred the development of such materials. Recent approaches have created hybrid materials that exhibit hierarchical spatial and chemical control at molecular and meso-length scales in two dimensions. Extending such precise control of biomineralization to mesoscale spherical templates remains a challenge. Particularly, a method allowing the patterning of hierarchical structures on polymeric substrates at a large scale and with the ease of tuning architecture and chemistry is lacking.

The present disclosure provides a novel and facile method to pattern mineral-based hierarchical morphology templated on polymer mesospheres, which are decorated with semiconductor nanocrystals by using an oil/water emulsion. The nanocrystals used herein function as nucleation sites for the induction of mineralization in addition to providing built-in imaging capabilities for further applications. Confined mineralization on the surface of mesospheres with finely-tuned mineral compositions results in a nanostructured shell with tunable architecture and chemistry. Furthermore, the mesospheres can potentially serve as reservoirs for hosting other molecules, such as therapeutic agents.

A novel and facile method is presented to fabricate inorganic shell/organic core hybrid materials with a hierarchical control over morphology and chemistry. For example, mesospheres composed of a model polymer, poly (lactic-co-glycolic acid) (PLGA), can be decorated with semiconductor nanocrystals by an oil/water emulsion and used as templates for the patterning of mineral-based hierarchical nanostructures. Characterization of these materials with scanning and transmission electron microscopy reveals mineral nanostructures distributed heterogeneously on the surface of the spherical template, with the semiconductor nanocrystals serving as nucleation sites for the induction of mineralization. By tailoring the composition of the mineral solution, nanostructures with distinct architecture and chemistry can be achieved. The practical and versatile patterning of various inorganic nanostructures on an organic spherical template offers a new class of hybrid nanomaterials, leading to a wide spectrum of applications.

In some aspects of the present disclosure, nanocomposites can be incorporated into tissue scaffolds, thereby improving the strength of the scaffold and resistance to degradation by the body, as well as to influence cellular behavior and biocompatibility. Prior studies have demonstrated that nanomaterials are more hydrophilic and possess an increased number of atoms and crystal grains at their surface compared to conventional materials. The large number of grains at the surface leads to increased surface roughness, surface area, and surface energy which are thought to contribute to an increase in protein adsorption and unfolding. For example, nanoscale ceramics, metals, and polymers have all been shown to improve cellular process compared to conventional materials. These properties make nanomaterials ideally suited to enhance the biocompatibility and cell/tissue interaction with tissue scaffolds such as extracellular matrix-derived scaffolds.

Generally, the size of the nanomaterials are selected to be substantially similar in size to the diameter of the fibers (e.g., collagen, elastin, fibronectin, laminin, glycosaminoglycans) in the tissue scaffold. In various embodiments, the nanoparticles have a mean diameter from about 5 nm to about 50 nm; from about 15 nm to about 30 nm; from about 15 nm to about 25 nm; or about 20 nm. In some aspects, the nanostructures (e.g., nanoparticles, nanorods, nanowires, nanofibers, or the like) have a mean diameter of from about 15 nm to about 45 nm; from about 20 nm to about 40 nm; from about 25 nm to about 35 nm; or about 30 nm. Further, the nanoparticles, nanorods, nanowires, or nanofibers can have a mean length of from about 100 nm to about 20 μm; from about 500 nm to about 20 μm; from about 1 μm to about 10 μm; or about 10 μm.

In various embodiments, the tissue scaffold alone or in the bionanocomposite retains its proteins, growth factors, and other peptides. For example, the tissue scaffold can retain growth factors such as vascular endothelial growth factor (VEGF), transforming growth factor (TGF-B 1), proteins such as collagen, elastic, fibronectin, and laminin, other compounds such a glycosaminoglycans. The tissue scaffold can thus release these factors during its remodeling and resorption by the body. This release is advantageous to cell growth and cell infiltration into the affected tissue. Therefore, retention of these compounds is advantageous for the implant material.

The nanocomposites of the present disclosure can be used to regulate cellular processes, which can be used in a variety of biological applications. Cellular processes include but are not limited to bone formation, protein synthesis, gene expression, cell proliferation, mitosis, DNA transcription, hormone production, enzyme production, cell death, gene delivery, or drug delivery.

Depending on the particular cellular function, and as circumstances vary, one of ordinary skill in the art would know how to assay cell function using methods known in the art. For example, in the case of gene expression and detecting a level of polynucleotide expression, any method for observing polynucleotide expression can be used without limitation. Such methods include but are not limited to traditional nucleic acid hybridization techniques, polymerase chain reaction (PCR) based methods, and protein determination. Absolute measurements of the expression levels need not be made, although they can be made. Thus, the present disclosure contemplates methods for comparing differences in expression levels between samples. Comparison of expression levels can be done visually or manually, or can be automated and done by a machine, using for example optical detection means. Hardware and software for analyzing differential expression of genes are available, and can be adapted for a particular gene. Likewise, nucleic acid hybridization techniques can be used to observe polynucleotide expression. Exemplary hybridization techniques include northern blotting, Southern blotting, solution hybridization, and 51 nuclease protection assays.

Similarly, cellular process can be assayed based on protein expression levels. Proteins can be observed by any means known in the art, including immunological methods, enzyme assays and protein array/proteomics techniques. Measurement of the translational state can be performed according to several protein methods. For example, whole genome monitoring of protein—the “proteome”—can be carried out by constructing a microarray in which binding sites comprise immobilized, preferably monoclonal, antibodies specific to a plurality of proteins. Methods for making polyclonal and monoclonal antibodies are well known, as described, for instance, in Harlow & Lane, ANTIBODIES: A LABORATORY MANUAL (Cold Spring Harbor Laboratory Press, 1988).

Alternatively, proteins can be separated by two-dimensional gel electrophoresis systems. Two-dimensional gel electrophoresis is well-known in the art and typically involves isoelectric focusing along a first dimension followed by SDS-PAGE electrophoresis along a second dimension. See, e.g., Hames et al., GEL ELECTROPHORESIS OF PROTEINS: A PRACTICAL APPROACH (IRL Press, 1990). The resulting electropherograms can be analyzed by numerous techniques, including mass spectrometric techniques, western blotting and immunoblot analysis using polyclonal and monoclonal antibodies, and internal and N-terminal micro-sequencing.

Likewise, cellular process can be assayed based by staining for cellular or morphological markers associated with a particular cellular function. For example, a cellular process like bone formation can be assayed by staining for calcium mineralization using Alizarin red. Also cellular process can be analyzed by proteomic or genomic assays by quantifying bio-reaction products or cellular proliferation, as known in the art.

The following examples are provided for the purpose of illustrating, not limiting, the invention.

### EXAMPLES

### Example 1

### The Preparation and Characteristics of Representative Strontium-Containing Colloidal Nanocomposites

In this example, the preparation and characteristics of representative strontium-containing colloidal nanocomposites is described.

Mineralizing Solutions.

The mineralizing solutions were prepared based on simulated body fluid (Table 1). All the agents of ACS grade were purchased from Sigma. All components except calcium chloride (CaCl₂) and strontium chloride (SrCl₂) were added together to Milli-Q water. Then CaCl₂ was added slowly with gentle stirring to prevent spontaneous precipitation. The resulting solution was buffered to pH 7.4 with Tris-HCl and filtered by a 0.2 μm pore-size filter. Prior to synthesis of the nanocomposites, a given volume of 1 M SrCl₂ aqueous solution was mixed into to the above solution to achieve the composition as shown in Table 1.

Synthesis of Colloidal Nanocomposites.

In 50 ml Falcon centrifuge tubes (BD Biosciences, San Jose, Calif.), 3 ml of mineral solution with different strontium concentrations (Table 1) was mixed with 15 μl DNA (1 mg/ml in Tris-EDTA) solution and 150 μl 20 kDa polyethylene glycol (PEG) (40%, w/v) aqueous solution. The resulting solution was placed on a Rotomix Type 50800 speed rotator (Barnstead/Thermolyne, Dubuque, Iowa) at the speed of 115 rpm and kept in an incubator at 37° C. After 24 h mineralization, the remaining PEG and mineral solution were removed by centrifuging the solution at 97 rcf for 90 min at 4° C. The nanocomposites were then re-suspended in serum free Dulbecco's Modified Eagle Media (DMEM) at the concentration of 13 μg plasmid DNA/ml. Nanocomposite suspensions were sonicated for 30 s in a water bath (Ultrasonic Cleaner Model B200, Cole-Parmer) right before adding to the cells.

Efficiency of DNA Incorporation.

After 24 h mineralization, 50 μl of the supernatants was sampled after the centrifugation at 97 rcf for 90 min at 4° C. and assayed for the concentration of DNA. The quantity of DNA in the supernatant was determined using the Quant-iT PicoGreen dsDNA reagent (Invitrogen, Carlsbad, Calif.) following the manufacturer's protocol. The amount of DNA precipitated was calculated by subtracting the amount of DNA remaining in the supernatant from the total DNA added to the mineralizing solution.

Size Measurements of Colloidal Nanocomposites.

The size of colloidal nanocomposites was determined using a Zetasizer Nano ZS (Malvern Instruments, Westborough, Mass.). Immediately after the synthesis of nanocomposites, 200 μl samples from the reaction solution were diluted with 800 μl of simulated body fluid for the size measurement. The size of colloidal nanocomposites after the centrifuge was also determined. The nanocomposites were precipitated by centrifuging at 97 rcf for 90 min at 4° C. and then re-suspended in simulated body fluid at the concentration of 4 μg DNA/ml and sonicated for 30 s in a water bath before the measurement. All measurements were carried out at 25° C.

Morphology and Composition of Colloidal Nanocomposites.

SEM was used to characterize the size and morphology of nanocomposites. The samples were sputter-coated with platinum using a SPI Sputter™ Coater (Structure Probe, Inc., West Chester, Pa.) and analyzed with a JEOL 7000 SEM with a beam voltage of 10 kV (Electron Microscopy Center, University of Washington). EDX was used to characterize the chemical compositions of nanocomposites. The samples were sputter-coated with carbon and analyzed with a beam voltage of 15 kV.

XRD of Colloidal Nanocomposites.

The crystallographic properties of colloidal nanocomposites were analyzed by a Bruker D8 Discover XRD with General Area Detector Diffraction Systems (GADDS) using Cu-Kα radiation (λ=1.54 Å) (Center for Nanotechnology, University of Washington). The diffractometer was operated at 40 kV and 120 mA. The focal distance between the lenses and the sample surface was set at 15 mm, and the sample depth was 1 mm. The scanning duration was 120 s per GADDS frame. Crystallographic information of nanocomposites was obtained by comparing the experimental XRD patterns to standards compiled by the Joint Committee on Powder Diffraction and Standards (JCPDS, HA: 00-009-0432; α-TCP: 00-009-0348; β-TCP: 00-009-0169; Sr-HAP: 00-014-0691; OCP: 01-074-1301). HA and β-TCP were the two major phases. The percentage of nanocomposites in different orientations for each crystal phase (hkl) was estimated using Equation (3):

$${\%}_{HA ( 211 )} = 100 \times \frac{{I}_{HA ( 211 )} / {I}_{HA ( 211 )}^{*}}{{\sum }_{hkl} ( {I}_{HA ( hkl )} / {I}_{HA ( hkl )}^{*} ) + K {\sum }_{hkl} ( {I}_{β \text{-} TCP ( hkl )} / {I}_{β \text{-} TCP ( hkl )}^{*} )} \\ I \text{:} measured intensity of peaks of each orientation \\ {I}^{*} \text{:} the intensity of standard XRD profile from JCPDS \\ K \text{:} the relative scattering power of β \text{-} TCP to HA \qquad ( 3 )$$

Percent crystallinity, the ratio of integrated intensity from the crystalline peaks to the sum of the crystalline and amorphous intensities, was used to determine the crystallinity of nanocomposites. The background from substrate, air, and incoherent scattering was removed.

The size of crystal was calculated by using Scherrer's equation, Equation (4). The peak of 2θ=31.8° was used to define the full width at half maximum intensity (B_{1/2}(rad)).

$$d = \frac{k λ}{{B}_{1 / 2} cos θ} \\ d \text{:} crystal size \\ λ \text{:} wavelength of Cu \text{-} K α radiation , λ = 0.154 nm \\ k \text{:} the broadening constant , k = 0.9 \qquad ( 4 )$$

Cell Culture.

MEFs were purchased from the Jackson Laboratory or ATCC. MEFs were maintained in Dulbecco's Modified Eagle Media (DMEM), supplemented with 10% FBS, 2 mM Glutamax, 1 mM sodium pyruvate, 100 units-100 μg/ml Penicillin-Streptomycin, 0.1 mM non-essential amino acids, and 150 μM monothioglycerol at 37° C. and 5% CO₂. The cells were used within 6 passages.

Formation of Complexes of DNA with Lipofectamine™ 2000 (Lipo).

The Lipofectamine-mediated gene transfer was performed following the manufacture's protocol. Briefly, 50 μl DMEM containing 4 μg of DNA was mixed with 50 μl DMEM containing 8 μl of Lipofectamine™ 2000. The mixture was incubated at room temperature for 20 min before adding to the cells. The complexes were called lipoplexes.

Gene Transfer Using Colloidal Nanocomposites.

MEFs in 500 μA tissue culture medium were directly plated to each well of 24-well tissue culture plates (BD Biosciences, San Jose, Calif.) at the density of 8×10⁴ cells per well. The cells were incubated at 37° C. and 5% CO₂ for 24 h. Then the colloidal nanocomposites containing 4 μg of DNA (gWIZ Beta-gal (Aldevron, Fargo, N. Dak.) encoding β-galactosidase (β-gal)) were added to the cells in 300 μA of serum free DMEM. 2 h later, the media was replaced with fresh tissue culture media and the cells were incubated at 37° C. and 5% CO₂ for an additional 24 h.

Gene Transfer Using Lipofectamine™ 2000.

MEFs in 500 μA of cell culture medium were directly plated to each well of 24-well tissue culture plates (BD Biosciences, San Jose, Calif.) at the density of 8×10⁴ cells per well. Cells were incubated at 37° C. and 5% CO₂ for 24 h. Then the cell culture medium was replaced with 500 μl of serum-free DMEM containing lipoplexes. 6 h later, the medium was replaced with fresh tissue culture media and the cells were incubated at 37° C. and 5% CO₂ for an additional 24 h.

Quantification of Gene Transfer Efficiency.

The cells were lysed with 150 μl of a solution containing 10 μM 2-mercaptoethanol, 9 mM MgCl₂, and 0.1% triton X-100 in D-PBS for 15 min. Then, three freeze-thaw cycles between −80° C. and 37° C. were performed to ensure the complete release of proteins from cells. 50 μA of the lysed cell solution was mixed with 50 μl solution containing 0.15 mM chlorophenol red-β-D-galactoside (CPRG), 10 μM 2-mercaptonethanol, 9 mM MgCl₂, and 0.1% Triton X-100 in D-PBS and incubated at 37° C. for 30 min. The absorption was measured at 570 nm using a SpectraMax M5 microplate reader (Molecular Devices, Sunnyvale, Calif.). The quantity of β-gal produced by the cells was determined by using a standard curve constructed with known concentrations of β-gal. Gene transfer efficiency was expressed as ng of β-gal per mg of total protein per well. Total protein was measured using the Coomassie protein assay (Biorad, Hercules, Calif.). Briefly, 5 μl of the lysed cell solution was diluted with 5 μl D-PBS and then mixed with 200 μl of Coomassie solution. The absorption at 595 nm was determined with the microplate reader. The quantity of protein was determined by using a standard curve with known concentration of bovine serum albumin (BSA).

Determination of the Metabolic Activity of Cells Transfected with Strontium-Containing Colloidal Nanocomposites.

The metabolic activity of cells transfected with strontium-containing colloidal nanocomposites was determined using the 3-(4,5-dimethylthiazol-2-yl)-2,5-diphenyltetrazolium bromide (MTT) colorimetric method, which is based on the reduction of the MTT salt into formazan crystals by viable cells. Briefly, 24 h post transfection, 50 μl of a 5 mg/ml aqueous solution of MTT (Sigma, St. Louis, Mo.) was added and incubated at 37° C. and 5% CO₂ for an additional 2.5 h. Following centrifugation, the supernatant was removed and 1 ml dimethyl sulfoxide was added to cells and incubated at 37° C. for 30 min to dissolve the as formed formazan crystals. The absorption at 570 nm was determined using a microplate reader.

Statistical Analysis.

Triplicate samples were included in all the experiments. The values shown were as the mean of triplicates. All experiments were repeated two to three times. Data were shown as means±s.e.m. Statistical significance of differences was determined by the two-tailed Student's t-test.

### Example 2

### Methods for Surface-Induced Mineralization, Mineralized Surfaces, and Characteristics and Uses of the Surfaces

In this example, methods for surface-induced mineralization, mineralized surfaces, and the characteristics and uses of the surfaces is described.

Formation of Surface-Induced DNA-Doped Nanocomposites.

All reagents for mineralization were purchased from Sigma. All mineral solution components, except CaCl₂, were added together in Milli-Q water and CaCl₂ was added last to prevent spontaneous precipitation. The resulting solutions was buffered to pH 7.4 with Tris-HCl and filtered by a 0.2 μm pore-size filter and stored at 4° C. One milliliter of a given mineral solution with different formulations (Table 1) was mixed with 10 μl of a DNA solution (100 μg/ml) and added to 24-well tissue-culture plates (BD Biosciences, San Jose, Calif.). The reporter plasmid, gWIZ Beta-gal (Aldevron, Fargo, N. Dak.), encodes for the reporter enzyme β-galactosidase (β-gal). The mineralization was conducted at 37° C. in a humidified incubator for an indicated period of time.

For formation of DNA-DOTAP-doped nanocomposites, 10 μl of DOTAP in Dulbecco's phosphate buffered saline (DPBS) (0.6 or 1.2 mg/ml) was mixed with 10 μl of DNA solution (100 μg/ml) in DPBS. The resulting DNA-DOTAP complex was incubated at room temperature for 15 min. One milliliter of a given mineral solution with different formulations (Table 5) was mixed with 20 μl of the DNA-DOTAP complex and added to each well of 24-well plates. The mineralization was conducted at 37° C. in a humidified incubator for an indicated period of time.

Morphology and Composition of Nanocomposites.

SEM was used to characterize the size and morphology of nanocomposites mineralized for 24 h. The samples were sputter-coated with 12 nm of platinum using a SPI Sputter™ Sputter Coater (Structure Probe, Inc.; West Chester, Pa.) and were analyzed with a JEOL 7000 SEM with a beam voltage of 10 keV (Electron Microscopy Center, University of Washington). EDAX was used to characterize the chemical compositions of nanocomposites. The samples were sputter-coated with carbon and analyzed with a beam voltage of 15 keV.

pH Responsiveness of DNA—doped Nanocomposites.

Mineralization was carried out as described above for the indicated durations. After mineralization, the solution was removed and 1 ml fresh mineral solution A (simulated body fluid) was added to each well. Dissolution profiles of different minerals were determined by gradually decreasing the pH of each well using 0.1 M hydrochloric acid. The volume of acid added was less than 20 μl for each pH point. Samples were shaken on a rotary mixer to insure quick and even distribution and allowed to equilibrate for 2 minutes before sampling. Calcium released from nanocomposites was determined using a modified colorimetric method based on complex formation with ortho-cresolphthalein (Morin, Am J Clin Pathol, 61(1):114-117 (1974)). The pH at which 50% of total calcium was released, designated pH₅₀, was used as an indicator of pH responsiveness. The dissolution was conducted at room temperature.

Determination of the Efficiency of DNA Incorporation.

During the course of mineralization, 50 μl of the supernatant in each well was sampled and assayed for the DNA concentration. The quantity of DNA in the supernatant was determined using the Quant-iT PicoGreen dsDNA reagent (Invitrogen; Carlsbad, Calif.) following the manufacturer's protocol. The amount of DNA precipitated was calculated by subtracting the amount of DNA remaining in the supernatants from the total DNA added to each well.

Cell Culture.

MG-63 cells were cultured in Dulbecco's Modified Eagle Media (DMEM), supplemented with 10% fetal bovine serum (FBS), 1% penicillin-streptomycin (P-S), 1% L-glutamine, and 1% sodium pyruvate; Saos-2 cells were cultured in DMEM, supplemented with 10% FBS, 1% P-S, and 2% L-glutamine; EMT6 cells were cultured in DMEM, supplemented with 10% FBS, 1% P-S, and 1% L-glutamine; Caco-2 cells were cultured in Modified Eagle Medium (MEM), supplemented with 20% FBS and 1% P-S; Ishikawa and Hela cells were cultured in MEM, supplemented with 10% FBS, 1% P-S, and 1% L-glutamine; TC1 cells were cultured in RPMI Media 1640, supplemented with 10% FBS, 1% HEPES, 1% sodium pyruvate, 1% L-glutamine, 0.15% sodium bicarbonate, and 0.45% glucose; B35 cells were cultured in DMEM, supplemented with 10% FBS and 1% L-glutamine; Hep G2 cells were cultured in MEM, supplemented with 10% FBS and 1% sodium pyruvate. All cells were maintained in an incubator at 37° C. and 5% CO₂.

Imaging of Cells with Bright-Field Light Microscopy.

Cells grown on surfaces coated with DNA-doped nanocomposites were monitored by a Nikon TE2000 inverted microscope with a 20× objective. Images were acquired with a CoolSnap ES2 charge-coupled device camera (Photometrics, Tucson, Ariz.).

Gene Transfer of Cells Cultured on DNA-Doped Nanocomposites.

1×10⁵ cells in 500 μl tissue culture medium were directly plated on surfaces coated with DNA-doped nanocomposites mineralized for 8 h for MG-63, Saos-2, Caco-2, Ishikawa, TC1, Hela, B35 and Hep G2; 5×10⁵ cells were plated for EMT6 per well. Cells were incubated at 37° C. and 5% CO₂ for 36 h before analysis of cell viability and transfection efficiency. A commercial transfection reagent, Lipofectamine 2000 (Invitrogen; Carlsbad, Calif.), was used as a positive control following the manufacturer's protocol.

Determination of Gene Transfer Efficiency.

Cells were grown on surfaces coated with DNA-doped nanocomposites for 36 h. The media was then removed, and samples were then lysed with 150 μl of a solution containing 10 μM 2-ME, 9 mM MgCl₂ and 0.1% triton X-100 in DPBS for 15 min. Then, three freeze-thaw cycles between −80° C. and 37° C. were performed to ensure complete release of proteins from cells. 50 μl of the lysed cell solution was mixed with 50 μl solution containing 0.15 mM chlorophenol red-β-D-galactoside (CPRG), 10 μM 2-ME, 9 mM MgCl₂, and 0.1% triton X-100 in DPBS and incubated at 37° C. and 5% CO₂ for 30 min. The absorption was measured at 570 nm using a SpectraMax M5 microplate reader (Molecular Devices, Sunnyvale, Calif.). The quantity of β-gal produced by the cells was determined by using a standard curve constructed with known concentrations of β-gal. Gene transfer efficiency was expressed as ng of β-gal per mg of total protein. Total protein was measured using the Coomassie protein assay (Biorad, Hercules, Calif.). Briefly, 5 μl of the lysed cell solution was diluted with 5 μA DPBS and then mixed with 200 μl of Coomassie solution. The absorption at 595 nm was determined with a microplate reader. The quantity of protein was determined by using a standard curve with known concentration of bovine serum albumin protein.

Determination of Metabolic Activity of Cells.

Metabolic activity of cells on DNA-doped nanocomposites was determined using the MTT colorimetric method, which is based on the reduction of the MTT salt into formazan crystals by viable cells. Herring sperm DNA (1 μg/ml) was used for mineralization. Briefly, cells were grown on surfaces coated with DNA-doped nanocomposites as above. 36 h later, 50 μl of a 5 mg/ml solution of MTT (Sigma, St. Louis, Mo.) in water was added and incubated at 37° C. and 5% CO₂ for an additional 2.5 h. Following centrifugation, the supernatant was removed and 1 ml dimethyl sulfoxide was added to cells and incubated at 37° C. for 30 min to dissolve the formazan crystals. The absorption of the wells at 570 nm was determined using a microplate reader.

Quantification of DNA Uptake by Flow Cytometry.

0.5 μg fluorescein isothiocyanate-labeled DNA (2.7 kilobase pairs) (Mirus Bio, Madison, Wis.) was co-precipitated with 1 ml mineral solution for 8 h. The solution was removed and cells were plated at the same densities as above. 24 h after cells were cultured on surfaces coated with DNA-doped nanocomposites, the plate was washed with 0.25 ml Hank's Balanced Salt Solutions (HBSS). Cells were detached using 250 μl of trypsin-EDTA (0.1% Trypsin, 0.4% EDTA.4Na) at 37° C. for 15 min. Cells were then washed with a solution containing 1% FBS in DPBS twice and analyzed by flow cytometry immediately using a BD FACScan2 (Cell Analysis Facility, Department of Immunology, University of Washington). Trypan blue was used to quench the fluorescence associated with the cell surface. The data was analyzed with FlowJo (Treestar, Ashland, Oreg.), and the arithmetic mean of fluorescence intensity (MFI) of each sample was determined. The uptake of DNA by cells was expressed as the MFI of cells cultured on surfaces coated with DNA-FITC-doped nanocomposites subtracted with the MFI of cells cultured on uncoated surface.

Measurement of Phagosomal pH.

100 nm beads with amine functional groups (Polysciences, Warrington, Pa.) were coupled with the pH-sensitive dye FITC and the pH-insensitive dye AlexaFluor 647 succinimidyl ester (AlexaFluor647) (Invitrogen, Carlsbad, Calif.). Beads were resuspended in 500 μl 0.1 M carbonate buffer with a pH of 9 at 1% w/v. 5 μA of FITC, and AlexaFlour647 from a 10 mg/ml stock in PBS were added to the beads and mixed. The beads were then incubated at 4° C. for 16 h, washed 3 times with PBS, and resuspended 500 μA PBS. Cells were incubated with beads for 15 min, washed 3 times with PBS, and further incubated at 37° C. in media for 15 min and immediately analyzed by flow cytometry. The ratio of the mean fluorescence intensity of FITC and AlexaFluor647 for each sample was determined (FL1/FL3 ratio). Values were calibrated against a standard curve obtained by resuspending cells that were exposed to coupled beads for 2 h. Cells used for obtaining calibration curves were fixed with 4% PFA, permeabilized with 0.1% triton X-100, incubated in citrate buffers with defined pH and immediately analyzed by flow cytometry.

Statistical Analysis.

Triplicate samples were included for all experiments and expressed as the average. Experiments were repeated at least two times. The standard deviation is within 5%, if not mentioned.

### Example 3

### Immobilization of Liposome/DNA Complexes by Surface Induced-Biomineralization for Enhanced Gene Transfer

Cell Culture and Media.

B35 cells were cultured in Dulbecco's Modified Eagle Media (DMEM), supplemented with 10% fetal bovine serum (FBS) and 2 mM L-glutamine. The cells were maintained in an incubator at 37° C. and 5% CO₂.

DOTAP Liposome Preparation.

A given amount of chloroform solution of DOTAP (10 mg/ml) was placed in a glass tube and the chloroform was evaporated under a stream of argon gas. For the direct mixing method, which has been used by previous studies (Zhang et al., J Pharmaceut Sci, 97(1):237-250 (2008); Wang et al., Mol Pharm, 4(4):615-623 (2007); Tabatt et al., J Control Release, 97(2):321-332 (2004)), the lipid film was re-suspended at 2 mg/ml in Dulbecco's phosphate buffered saline (D-PBS) at room temperature and then directly used for the complexation with DNA. For the freeze-thaw-extrusion method, the lipid film was re-suspended at 2 mg/ml in HEPES-buffered saline (20 mM HEPES, 150 mM NaCl, pH 7.4) and incubated at 43° C. for 1 h. The resulting solution was subjected to freeze (−80° C.)-thaw (40° C.) cycles for 10 times, then passed through the extruder (Avanti Polar Lipids; Alabaster, Ala.) with a polycarbonate membrane filter (100 and 1000 nm) five times before the complexation with DNA.

Characterization of DNA/DOTAP Lipoplexes.

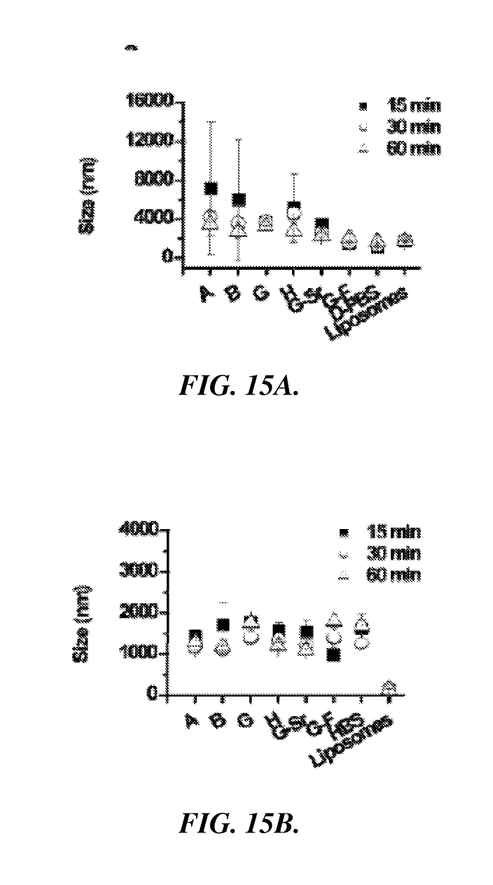

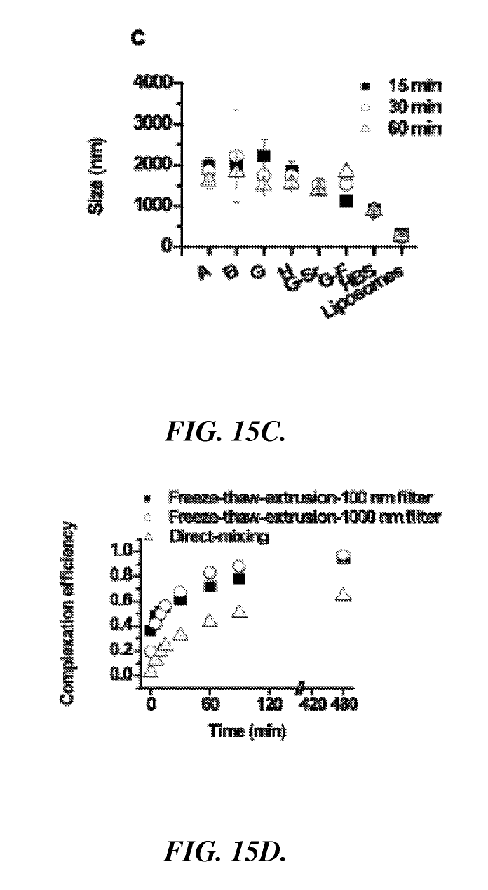

The size and structure of liposomes can potentially affect the size and stability of lipoplexes and thus the size, morphology and reproducibility of nanocomposites. Initially, the methods to prepare DOTAP liposomes were explored. Two methods were examined. In method 1, called the direct-mixing method, liposomes were formed by directly mixing dried lipids with aqueous solutions via vortexing. This method has been commonly used by other studies (Zhang et al., J Pharmaceut Sci, 97(1):237-250 (2008); Wang et al., Mol Pharm, 4(4):615-623 (2007); Tabatt et al., J Control Release, 97(2):321-332 (2004)) and resulted in multi-lamellar structures. Using the direct-mixing method, the mean diameter of the DOTAP liposomes was around 1800 nm with polydispersity of 0.28. Upon the complexation with DNA, the size of lipoplexes varied dramatically within a 60-min incubation and significant aggregations indicated by the turbidity were observed in the presence of mineral solutions (FIG. 15A). The aggregation and instability of lipoplexes can potentially affect the reproducibility of surface-induced nanocomposites.

In method 2, called freeze-thaw-extrusion method, the liposomes were subjected to freeze-thaw cycles and then passed through filters with pore sizes of 1000 nm or 100 nm in diameter. The size of liposomes before the extrusion is 324 nm in diameter with a polydispersity of 0.36. The 1000 nm filter resulted in liposomes with an average diameter of 257 nm and polydispersity of 0.23 while the 100 nm filter resulted in liposomes with an average diameter of 129 nm and polydispersity of 0.16. After the complexation with DNA, the size of lipoplexes increased to around 1500 nm and 2000 nm in diameter for 100 and 1000 nm filters, respectively. The lipoplexes were stable in most of the mineral solutions examined (FIGS. 15B and 15C).

DNA complexation efficiency of DOTAP liposomes made by both methods was determined. For the direct-mixing method, the complexation efficiency gradually reached 40% within one hour and then leveled off (FIG. 15D). For the freeze-thaw-extrusion method, the DNA more efficiently complexed with liposomes and 70% complexation was achieved within one hour for both filter sizes. Based on the stability of liposomes and lipoplexes and the complexation efficiency, the freeze-thaw-extrusion method was chosen to make liposomes and 15 min complexation unless specified for the following studies to reduce potential variations resulting from the fabrication of liposomes.

Size and Stability Measurements.

The size of liposomes and lipoplexes were examined using Zetasizer Nano S90 (Malvern Instruments; Westborough, Mass.). The measurements were carried out at 25° C. in the indicated solution. The size changes with the time and is used to evaluate the stability of liposomes and lipoplexes.

DNA Complexation Efficiency.

25 μl of 1 μg/ml pcDNA3-LDHC4 was mixed with 100 μl of 1:500 dilution of Quant-iT PicoGreen dsDNA reagent (Invitrogen; Carlsbad, Calif.) for 5 min. The resulting solution was then mixed with 25 μl of 12 μg/ml DOTAP liposomes to yield DNA to DOTAP ratio of 1:12 (w/w). As the DNA was complexed with liposomes, the fluorescence intensity decreased. The fluorescence change of the resulting solution at 520 nm was recorded (excitation wavelength: 480 nm) using a SpectraMax M5 microplate reader (Molecular Devices; Sunnyvale, Calif.). A control, in which there were no liposomes, was also included to take into account the change of fluorescence due to the prolonged incubation of dye with DNA and photo-bleaching. The amount of DNA complexed with DOTAP was calculated by subtracting the free DNA remaining in the solution from the total DNA added to the solution.

Characterization of Lipoplex-Nanocomposites.

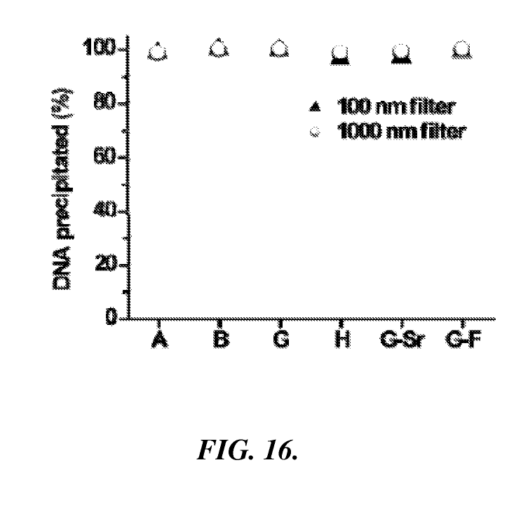

Initially, the efficiency of immobilization of lipoplexes was determined by surface-induced biomineralization. Regardless of the composition of mineral solutions and initial size of DOTAP, nearly 100% DNA in the form of lipoplexes was efficiently immobilized into the thin films of biominerals within the 8 h mineralization (FIG. 16). Surface-induced biomineralization efficiently immobilized DNA onto the surfaces in the form of either naked DNA or lipoplexes.

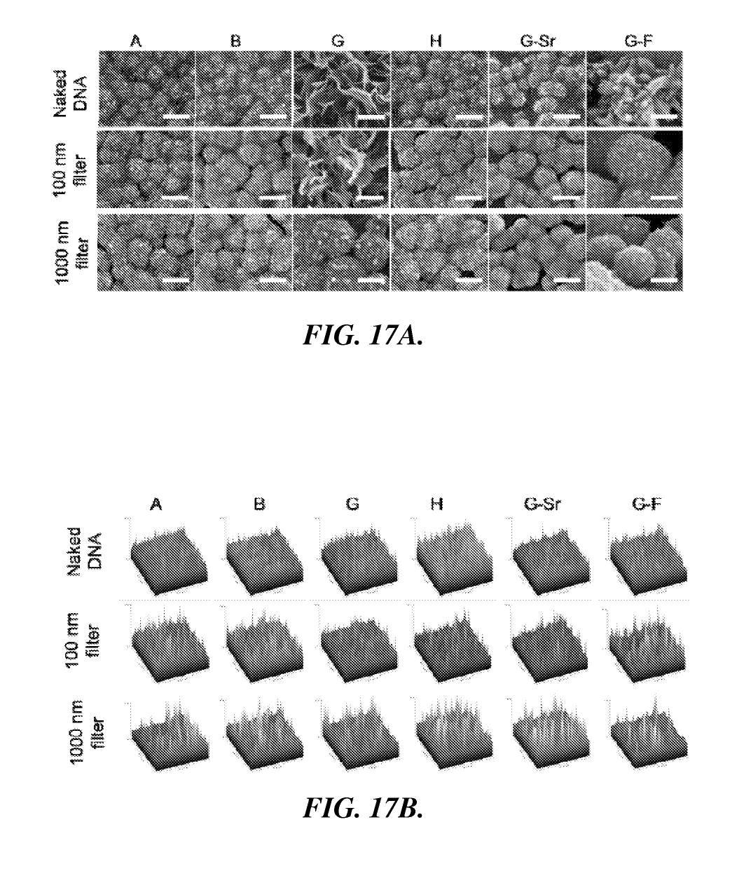

One goal was to control the architecture of nanocomposites so that more DNA can be accessible to cells for effective gene transfer. SEM was used to examine the architecture of surface-induced nanocomposites. As shown in FIG. 17A, nanocomposites derived from naked DNA formed a continuous thin film, while nanocomposites from lipoplexes exhibited a more segregated, defined spherical structure. For nanocomposite G and G-F, lipoplexes resulted in a dramatic morphological change of nanocomposites. Nanocomposite G formed from smaller liposomes, where 100 nm filters were used, exhibited a thickened plate-like structure. In contrast, nanocomposite G transformed from a continuous plate-like structure to a segregated spherical structure when it is formed from larger liposomes. Nanocomposite G-F transformed from a randomly distributed needle-like structure into a more uniformly distributed spherical structure. The results showed that the lipoplexes modulated the architecture of nanocomposites on the surface of substrates.

The DNA distribution on the surface of substrates was subsequently examined by fluorescent microscope. A 3-D surface plot of the fluorescence intensity of FITC-labeled DNA showed that, for all the mineral formulations examined, DNA was organized into discrete depots when DNA was first complexed with DOTAP before the induction of mineralization in comparison with the diffused patterns when naked DNA was used (FIG. 17B). For larger liposome sizes, larger discrete depots of DNA were formed. During the preparation of liposomes, for different sizes of filters, the amount of DOTAP and DNA used was kept the same. However, more DOTAP liposome particles and thus less DNA molecules per liposome were formed from 100 nm-than 1000 nm-filter. Therefore, DNA molecules were more localized and segregated on the surface deposited with nanocomposites derived from larger liposomes. These results have demonstrated that lipoplexes induce the architectural changes of surface-induced nanocomposites and organize DNA into more discrete depots.

Surface Induction of DNA- or Lipoplex-Nanocomposites.

Briefly, one milliliter of mineral solutions (Table 4) with different formulations was mixed with 1 μg of DNA either in the form of naked DNA (10 μl of 100 μg/ml DNA in Tris-EDTA) or DNA/DOTAP complexes (lipoplexes, 20 μl in milli-Q water). The mixtures were added to each well of 24-well tissue culture plates (BD Biosciences; San Jose, Calif.). The mineralization was conducted at 37° C. in a humid incubator for 8 h. The resulting nanocomposites were named after the respective mineral composition shown in Table 4.

Quantification of the Efficiency of DNA Deposition.

During the course of mineralization, 50 μl of the supernatant in each well was sampled and assayed for the DNA concentration by Quant-iT PicoGreen dsDNA reagent (Invitrogen; Carlsbad, Calif.) following the manufacturer's protocol. The amount of DNA precipitated was calculated by subtracting the amount of DNA remaining in the supernatants from the total DNA added. The standard curve was constructed by using lipoplexes formed under the same conditions as those used for mineralization to take into account the effect of complexation on the fluorescent intensity. DNA or lipoplexes in DPBS was included as a control and incubated at 37° C. as samples for mineralization. The concentration of DNA before and after the incubation was tested. There was no DNA or lipoplexes bound to the sides of wells in the absence of minerals.

DNA Organization on the Surface Deposited with Nanocomposites.

Fluorescein isothiocyanate (FITC)-labeled DNA (2.7 kb) (Minis Bio; Madison, Wis.) was used. DNA- or lipoplex-nanocomposites were fabricated as above. DNA distribution was examined with a 20× objective by a Nikon TE 2000 inverted microscope. Images were acquired with a CoolSnap ES2 charge-coupled camera (Photometrics; Tucson, Ariz.). The surface plot of the fluorescence intensity of FITC-labeled DNA was generated by Image J (NIH).

Quantification of DNA Uptake by Flow Cytometry.

0.5 μg naked FITC-labeled DNA (2.7 kb) (Minis Bio; Madison, Wis.) or lipoplexes (0.5 μg DNA, DNA:DOTAP=1:12 (w/w)) were immobilized on the surfaces as described above. Cells were cultured on surfaces deposited with DNA- or DNA/DOTAP nanocomposite. 24 h later, cells were washed with 0.25 ml Hank's Balanced Salt Solutions (HBSS). Cells were detached using 250 μl of trypsin-EDTA (0.1% Trypsin, 0.4% EDTA.4Na) at 37° C. for 5 min. Cells were then washed twice with FACS (1% FBS in D-PBS) buffer and analyzed by flow cytometry immediately using a BD FACScan2 (Cell Analysis Facility, Department of Immunology, University of Washington). Trypan blue was used to quench the fluorescence associated with the cell surface. The data was analyzed with FlowJo (Treestar; Ashland, Oreg.), and the arithmetic mean of fluorescence intensity (MFI) of each sample was determined. The uptake of DNA by cells was expressed as the MFI of cells cultured on DNA- or lipoplex-nanocomposites subtracted with the MFI of cells cultured on cell culture surfaces free of nanocomposites.

Size and Morphology of Lipoplex-Nanocomposites.

Scanning electron microscope (SEM) was used to examine the size and morphology of surface-induced nanocomposites. The samples were sputter-coated with 12 nm of platinum using a SPI Sputter™ Sputter Coater (Structure Probe, Inc.; West Chester, Pa.) and were analyzed with a JEOL 7000 SEM with a beam voltage of 10 kV (Electron Microscopy Center, University of Washington).

Biocompatibility of Lipoplex-Nanocomposites.

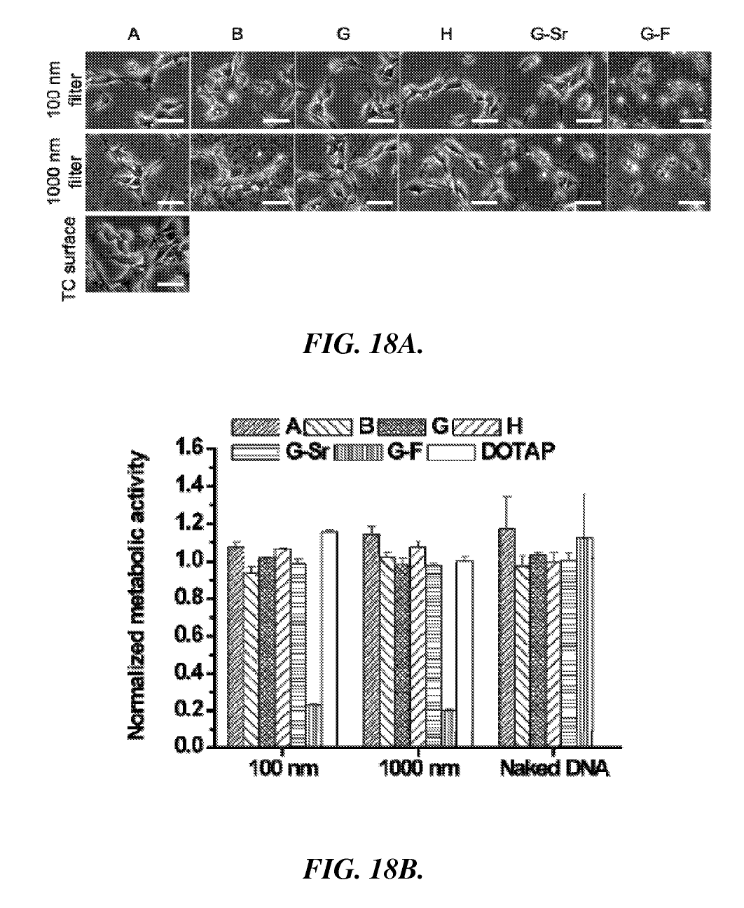

To ensure architectural changes of nanocomposites did not result in dramatic changes of biocompatibility of nanocomposite-deposited surfaces, the morphology and metabolic activity of cells cultured on these surfaces were assessed. For both filter sizes and most of mineral solutions examined except for formulation G-F, B35 cells on nanocomposite-deposited surfaces could attach and spread out as well as on surfaces free of nanocomposites (FIG. 18A). In addition, the cells on surfaces coated with lipoplex-nanocomposites exhibited similar metabolic activity as cells on surfaces free of nanocomposites (FIG. 18B).

Cellular Uptake of DNA.

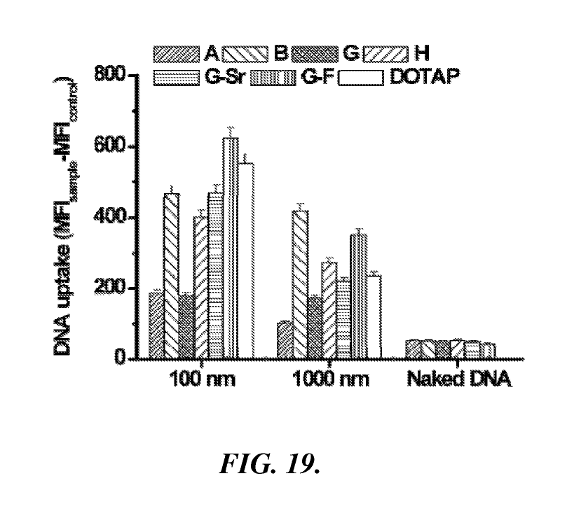

One hypothesis had been that controlling the architecture of nanocomposites on biomaterial surfaces to which cells were exposed could potentially enhance the cellular uptake of DNA and thus gene transfer efficiency. DNA cellular uptake in neuronal cells was examined. Clearly, for all the mineral compositions and both sizes of liposomes examined, immobilized lipoplexes resulted in 2 to 14 times more cellular uptake of DNA than that of the immobilized naked DNA (FIG. 19). Among all the formulations, nanocomposites formed from formulation B, H, G-Sr and G-F resulted in a more dramatic increase of cellular uptake of DNA than those from formulation A and G. This may be due to the architectural changes of nanocomposites. Lipoplex-nanocomposites derived from B, H, G-Sr and G-F seemed to be more segregated and easy to detach from surfaces and taken up by cells. The initial liposome size also affected the cellular uptake of DNA; larger size of liposomes resulted in a reduced level of cellular uptake of DNA, which was more apparent for nanocomposites from H, G-Sr and G-F.

Gene Transfer.

Cells in 500 μl tissue culture medium were directly plated on the surface deposited with DNA- and lipoplex-nanocomposites at the density of 1×10⁵ cells per well of a 24-well tissue culture plate and incubated at 37° C. and 5% CO₂. The reporter plasmid, gWIZ Beta-gal (Aldevron; Fargo, N. Dak.) encoding β-galactosidase (β-gal), was used.

36 h later, cells were lysed with 150 μl of a solution containing 10 μM 2-ME, 9 mM MgCl₂ and 0.1% triton X-100 in D-PBS for 15 min. Then, three freeze-thaw cycles between −80° C. and 37° C. were performed to ensure the complete release of proteins from cells. 50 μl of the lysed cell solution was mixed with 50 μl solution containing 0.15 mM chlorophenol red-β-D-galactoside (CPRG), 10 μM 2-ME, 9 mM MgCl₂ and 0.1% triton X-100 in D-PBS and incubated at 37° C. for 30 min. The absorption was measured at 570 nm using a SpectraMax M5 microplate reader. The quantity of β-gal produced by the cells was determined by using a standard curve constructed with known concentrations of β-gal. Gene transfer efficiency was expressed as ng of β-gal per mg of total protein. Total protein was measured using the Coomassie protein assay (Biorad; Hercules, Calif.). Briefly, 5 μl of the lysed cell solution was diluted with 5 μA D-PBS and then mixed with 200 μl of Coomassie solution. The absorption at 595 nm was determined with a microplate reader. The quantity of protein was determined by using a standard curve with known concentrations of bovine serum albumin (BSA).

Gene Transfer Efficiency.

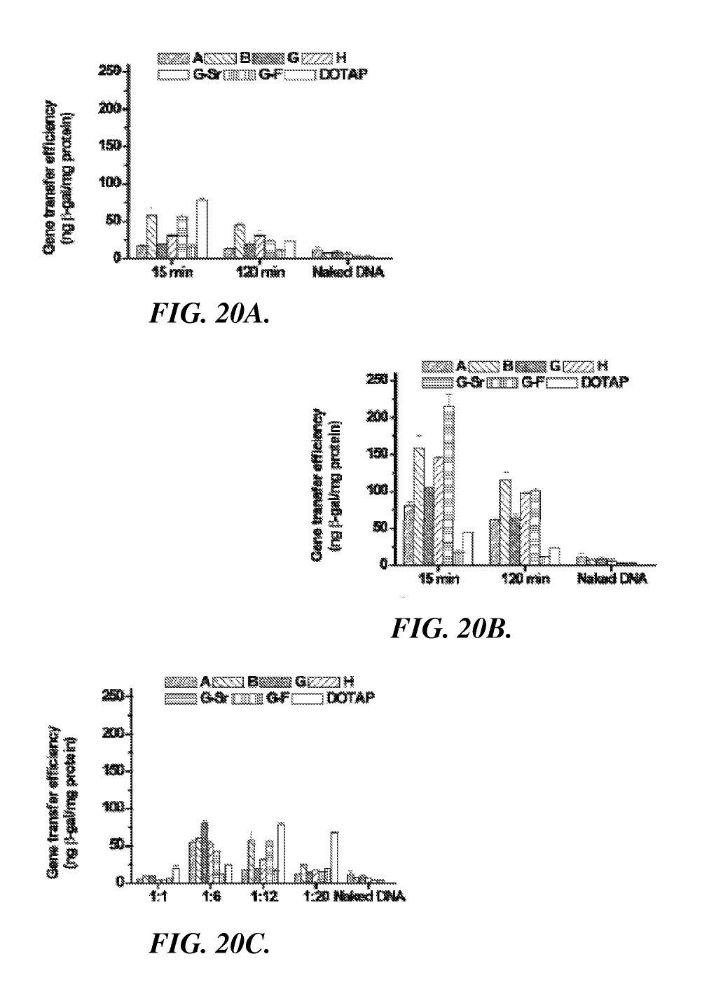

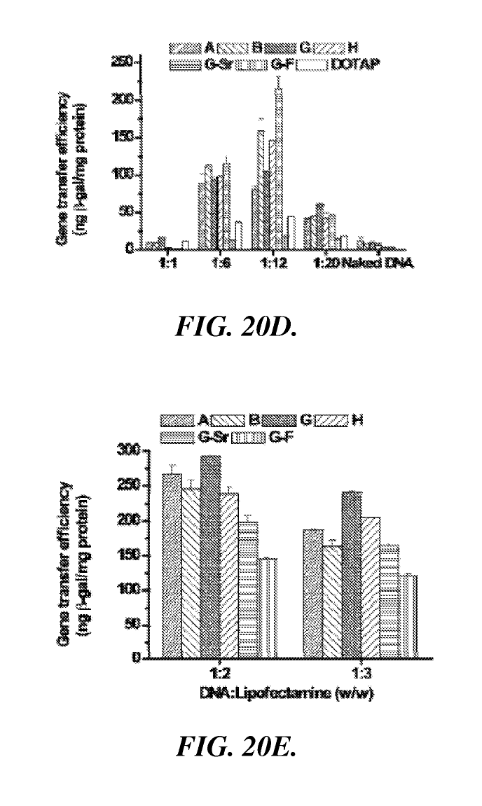

The gene transfer in neuronal cells, B35, was achieved by either immobilized lipoplex- or DNA-nanocomposites. Compared to immobilized naked DNA, immobilized lipoplexes by mineral solutions A, B, G, H, G-Sr resulted in significant increase in transgene expression, and the transgene expression varied depending on the composition of nanocomposites. For a given composition, the transgene expression can be further optimized by the initial size of liposomes, the complexation duration of DNA-liposome and the DNA to DOTAP ratio (FIG. 20).

The initial size of DOTAP has a significant effect on the level of transgene expression. Regardless of the composition of nanocomposites and the complexation duration, nanocomposites formed with larger liposomes resulted in a significant higher level of transgene expression than those from smaller liposomes. As demonstrated in FIGS. 15B and 15C, liposomes formed using 1000 nm filters resulted in lipoplexes larger than 100 nm ones. Larger lipoplex results in higher gene transfer efficiency either because larger lipoplexes lead to higher lipoplex-cell uptake and fusion or the formation of larger intracellular vesicles, which are easily disrupted for the release of DNA into cytoplasm. In addition, encapsulation of DNA within larger structures can better shield it from degradation by DNases. It appeared that the gene transfer in B35 cells by biomineral immobilized lipoplexes displayed similar lipoplex size-dependency as free lipoplexes for a given composition of nanocomposites and complexation duration. As discussed above, the overall cellular uptake of DNA was in general lower for 1000 nm-lipoplex than 100 nm-lipoplex nanocomposites, which indicated that immobilized lipoplexes can enter cells as the form of nanocomposites and the degree of uptake is controlled by properties of nanocomposites. The lower gene transfer efficiency by free lipoplexes derived from 1000-filter further supported that immobilized lipoplexes did not enter cells as free lipoplexes. Upon the entry of cells, nanocomposites can dissolve to free lipoplexes due to the progressive acidification of phagosomes in which they resided. As a result, for a given composition of nanocomposites, the level of gene transfer efficiency mediated by lipoplex-nanocomposites can be largely due to the facile release of DNA for larger liposomes as free lipoplexes.

The duration of complexation between DNA and liposomes and the effect the gene transfer efficiency of lipoplex-nanocomposites was considered. The results showed that longer complexation duration usually resulted in lower gene transfer efficiency (FIGS. 20A and 20B) than the shorter one. Previous studies have demonstrated that longer complexation duration can result in larger lipoplexes, which could increase the gene transfer efficiency (Ross et al., Gene Ther, 6(4):651-659 (1999)). In addition, the lipoplex structure continues to evolve after the initial binding event of DNA with liposomes (Zhang et al., Biochim Biophys Acta Biomembr, 1614(2):182-192 (2003)); longer complexation duration results in lipoplexes with structures containing several semi- or totally fused liposomes, so called “spaghetti-meatball assemblies” (Sternberg et al., FEBS Lett, 356(2-3):361-366 (1994)). Different structures of lipoplexes exhibit different ability of fusing with intracellular vesicles and releasing of DNA (Lin et al., J Drug Target, 8(1):13-27 (2000); Lin et al., Biophys J, 84(5):3307-3316 (2003)). As demonstrated in FIGS. 15B and 15C, the lipoplex size was stable over the time course examined. Therefore, it is likely the structure of the lipoplexes or the interactions between liposomes and DNA could possibly play a more critical role in the efficiency of gene transfer than the effect of sizes of lipoplexes.

The gene transfer efficiency by DNA/DOTAP nanocomposites was also mediated by the ratio of DNA to DOTAP (w/w) at both initial sizes of DOTAP for a given mineral composition (FIGS. 20C and 20D); the effect is more apparent for larger size of DOTAP liposomes with an optimal ratio of DNA to liposome at 1:12 (w/w) corresponding to a charge ratio of 5. As demonstrated in previous studies, for the liposome-mediated gene transfection, the transfection activity peaked at certain charge ratios and decreased with higher or lower charge ratios (Xu et al., Biophys J, 77(1):341-353 (1999); Reimer et al., J Biol Chem, 272(31):19480-19487 (1997); Lee et al., Hum Gene Ther, 7(14):1701-1717 (1996)). The immobilized lipoplexes exhibited a similar trend as free lipoplexes. This result further demonstrated that lipoplexes were freed from nanocomposites upon their entry of cells; the efficiency of gene transfer is thus further controlled by the properties of lipoplexes.

Lastly, the gene transfer efficiency by DNA/Lipofectamine™ 2000 nanocomposites was considered. Because the composition of lipids was unknown, two ratios were tested based on the manufacturer's instructions. The size of liposomes was 114.23±8.40 nm, and the size of the resulting lipoplexes was 496.90±102.67 for 1:2 and 220.60±0.57 nm for 1:3 of DNA to Lipofectamine ratio (w/w). As shown in FIG. 20E, the effect of charge ratio and composition of nanocomposites in the efficiency of gene transfer were similar to DNA/DOTAP nanocomposites. Therefore, the results derived from DOTAP liposomes can be generalized for other liposomes.

The results demonstrate that the immobilization of DNA as lipoplexes through surface-induced biomineralization significantly improved the gene transfer efficiency for neuronal cells. Lipoplexes modulated the architecture of nanocomposites on the surface and resulted in enhanced cellular DNA uptake. The possible pathway by which immobilized lipoplexes mediate the gene transfer was: DNA entered cells in the form of nanocomposites comprised of biomineral-decorated lipoplexes; in acidic phagosomal compartments, biominerals were dissolved and lipoplexes were freed from nanocomposites; subsequently, DNA escaped from phagosomal compartments in such a way as free lipoplexes, and then entered nuclei for transcription. Through this pathway, the gene transfer efficiency mediated by lipoplex-nanocomposites can be tuned by manipulating both the composition of biominerals and the physiochemical properties of lipoplexes.

Determination of Metabolic Activity of Cells.

Metabolic activity of cells on DNA- or lipoplex-nanocomposites was determined using the 3-(4,5-dimethylthiazol-2-yl)-2,5-diphenyltetrazolium bromide (MTT) colorimetric method, which is based on the reduction of the MTT into formazan crystals by viable cells. Herring sperm DNA (1 μg/ml) was used for mineralization. Briefly, cells were seeded on surface deposited with DNA/DOTAP or DNA nanocomposites as above. 36 h later, 50 μl of a 5 mg/ml aqueous solution of MTT (Sigma; St. Louis, Mo.) was added to cells and incubated at 37° C. and 5% CO₂ for an additional 2.5 h. Following centrifugation, the supernatant was removed and 1 ml dimethyl sulfoxide (DMSO) was added to cells and incubated at 37° C. for 30 min to dissolve the formazan crystals. The absorption of each sample at 570 nm was determined using a microplate reader. The relative cell metabolic viability was calculated by normalizing OD values of each sample to the value of cells on surfaces free of nanocomposites.

Statistical Analysis.

Triplicate samples were included in all the experiments. The values shown were as the mean of triplicates. All experiments were repeated two to three times. The standard deviation is within 5%, if not mentioned.

### Example 4

### Facile Patterning of Hierarchical Structures with Controlled Architecture and Chemistry on Polymer Mesospheres

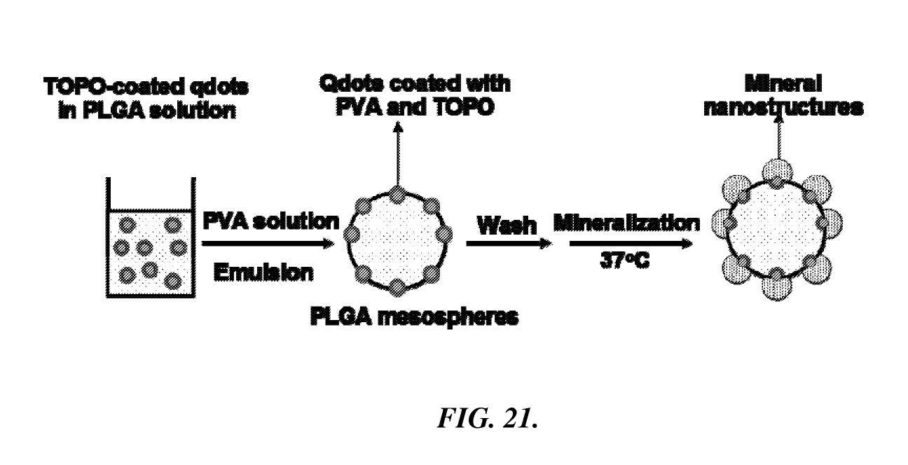

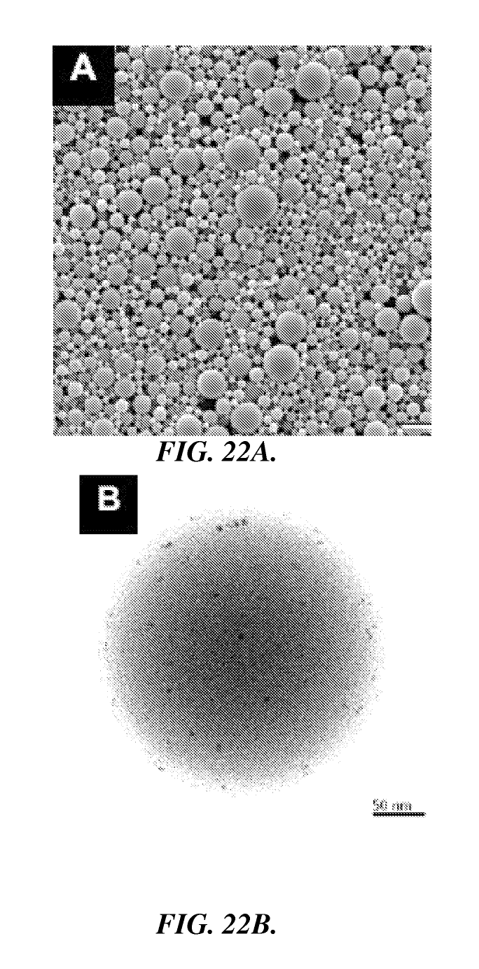

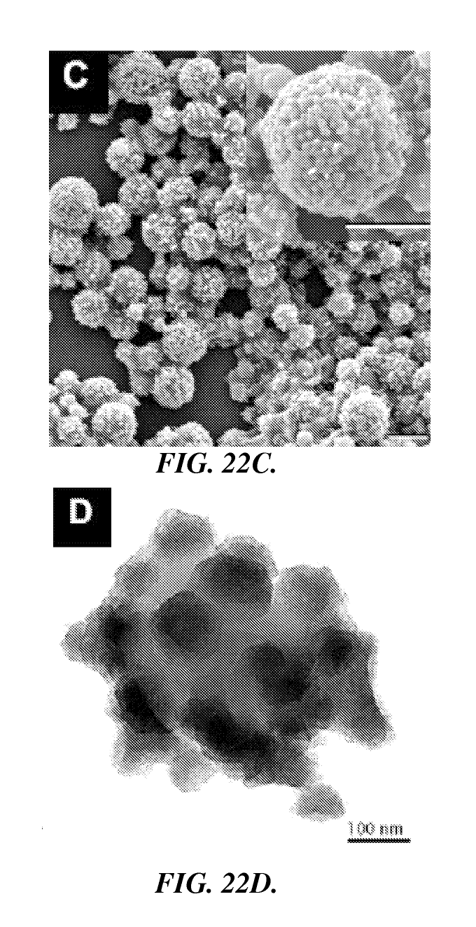

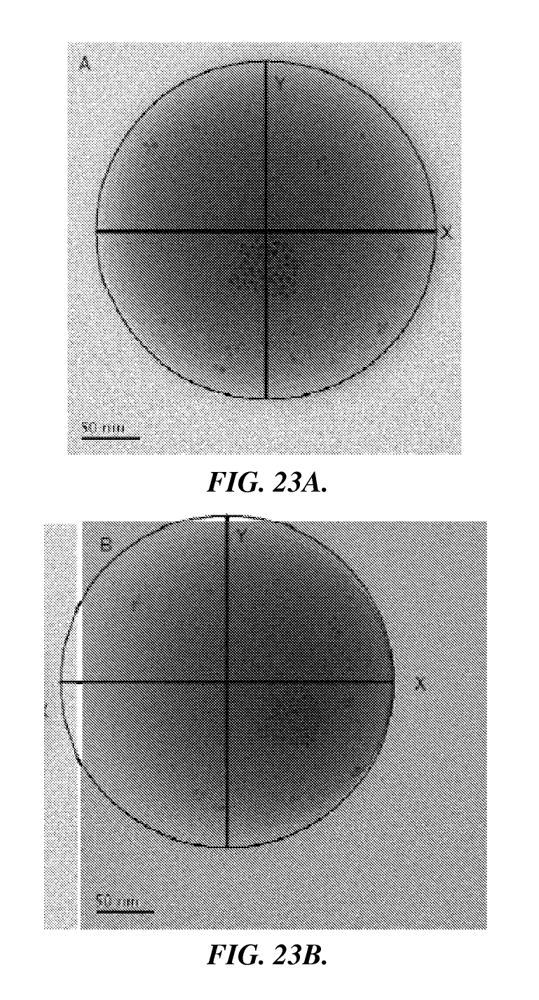

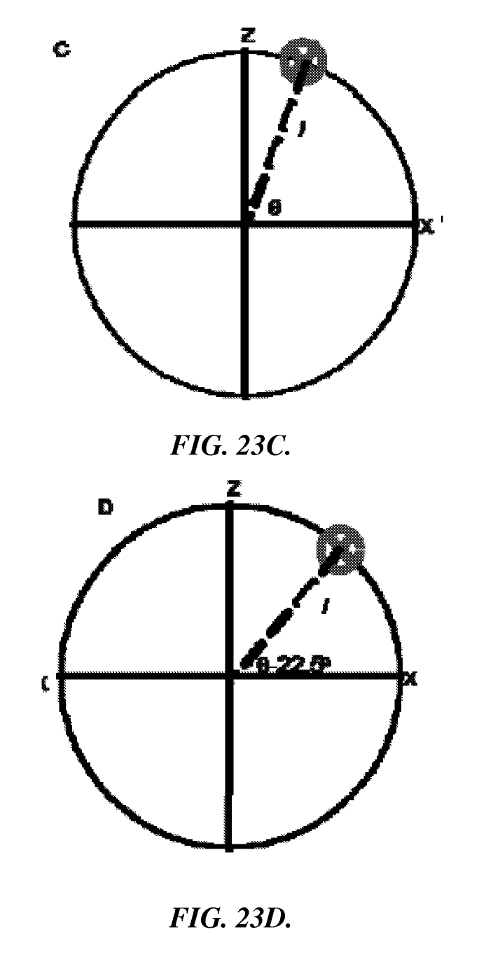

CdSe/ZnS quantum dots, capped with tri-n-octylphosphine oxide (TOPO), were doped on the surface of poly (lactic-co-glycolic acid) (PLGA) mesospheres using an oil/water emulsion. PLGA was used as a model polymer system in this study due to the ease of fabricating PLGA mesospheres and their wide applications in drug delivery and tissue engineering. If desired, other polymer templates can be used. Sonication was initially used to disperse quantum dots within the polymer solution. Upon addition of an aqueous solution containing a water-soluble surfactant, polyvinyl alcohol (PVA), quantum dots became entrapped in the organic phase containing the polymer. PVA not only stabilized the organic phase in the aqueous solution but also could partially replace TOPO or non-covalently interact with TOPO on the surface of quantum dots. As a result, quantum dots were coated with segregated regions of TOPO and PVA and preferentially remained at the oil/water interface (FIG. 21). After evaporation of the organic solvent, PLGA mesospheres solidified with quantum dots trapped on the surface (FIGS. 22A and 22B). It was confirmed that quantum dots were situated on or near the surface of mesospheres by determining the position of quantum dots based on transmission electron micrographs (TEMs) taken at both normal and tilted angles (FIGS. 23A-23D). The density of quantum dots decorated on the surface of mesospheres can be potentially tuned by controlling the initial amount of quantum dots added and the ratio of TOPO to PVA. This versatile method resulted in spherical polymer templates decorated with quantum dots, which can serve as nucleation sites for the induction of mineralization.

Quantum dot-doped PLGA mesospheres were fabricated using a modified oil-in-water (o/w) emulsification and solvent evaporation technique. TOPO-capped, core-shell CdSe/ZnS quantum dots with a photoluminescence emission maximum at 620 nm were purchased from Evident Technologies. 100 μl of quantum dot solution was added to 1 ml of 100 mg/ml PLGA solution in dichloromethane and then sonicated with a Branson Sonifier 450 for 10 sec at constant duty cycle. An o/w emulsion was formed by adding 2 ml of 1% polyvinyl alcohol (PVA) dropwise to the organic phase while vortexing. This emulsion was sonicated for 10 sec and then poured into 4 ml of 1% PVA while vortexing. Finally, the emulsion was poured into 4 ml of 0.06% PVA in a beaker. The resulting particle suspension was magnetically stirred for 4 h at room temperature. Particles were collected, washed three times with distilled water, and freeze-dried before analysis and mineralization.

Three mg of quantum dot-doped PLGA mesospheres were mixed with 3 ml of SBF or SBF-G containing a given concentration of PEG (MW 4000, Sigma), made as previously described (Shen, H.; Tan, J.; Saltzman, W. M. Nat. Mater. 2004, 8, 569) in a 50 ml polypropylene tube. Mineralization was carried out in a water bath shaker at 37° C. at 225 rpm for 2 d.

SEM was used to characterize the size and morphology of patterned polymer mesospheres. SEM samples were prepared by spin-coating a particle solution onto a piece of silicon wafer and dried overnight. The samples were sputter-coated with 10 nm of platinum using a Gatan Precision Etching and Coating System (Pleasanton, Calif.). Samples were analyzed with a JEOL 7000 SEM with a beam voltage of 5 keV (Electron Microscopy Center, University of Washington). For EDX, samples were sputter-coated with carbon and the chemical composition of the minerals was obtained with a beam voltage of 10 keV.

TEM samples were prepared by adding a drop of particle solution onto a formvar/carbon, 300 mesh copper grid (Ted Pella, Redding, Calif.), left to settle for 30 seconds and then blotted with filter paper. Samples were analyzed using a FEI Tecnai F20 equipped with a field emission gun (FEG) and operated at 200 kV (Yale University).

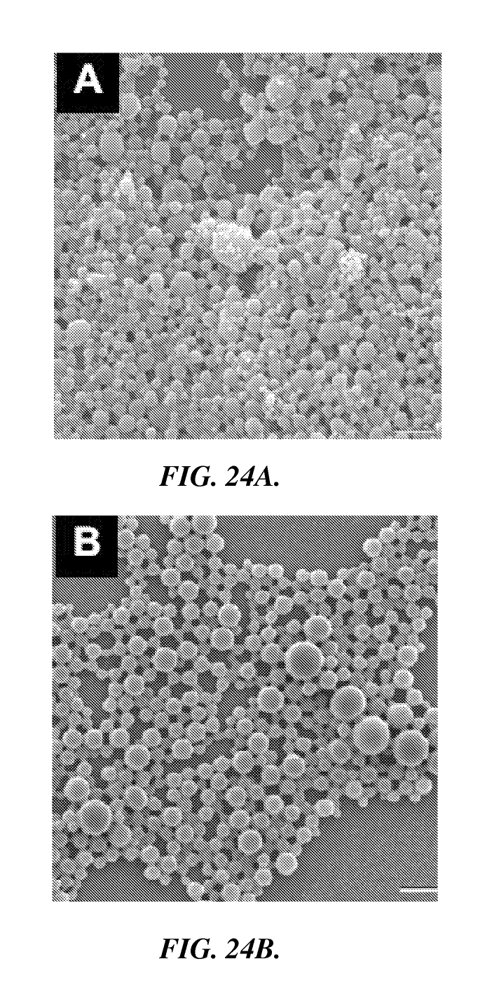

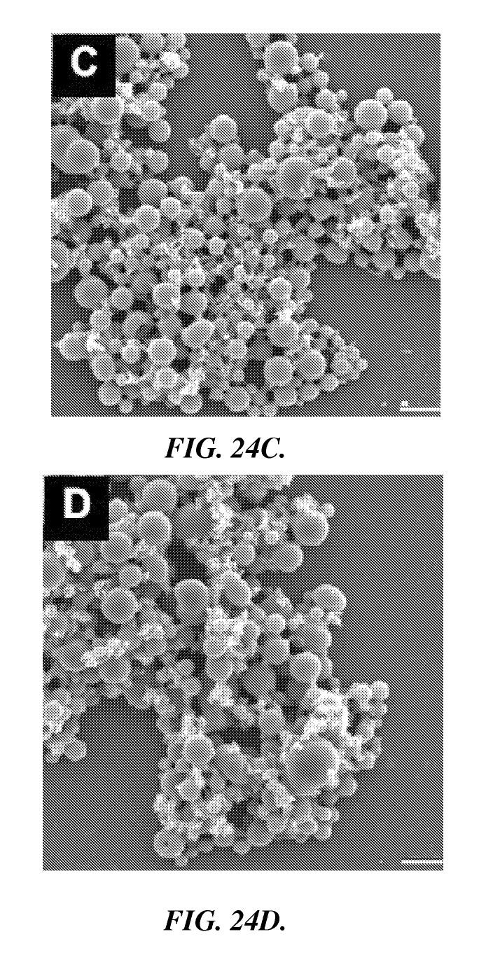

The mineralization process was initially carried out using a mineral solution containing 2.5 mM CaCl₂, 1 mM KH₂PO₄, 141 mM NaCl, 4 mM KCl, 0.5 mM MgSO₄, 1 mM MgCl₂ and 4.2 mM NaHCO₃. The composition of this solution is similar to that of body fluid, and so termed simulated body fluid (SBF). SBF is a supersaturated solution with respect to apatites and readily forms calcium phosphate-based mineral structures. PLGA mesospheres were incubated in SBF at 37° C. for two days with a given concentration of polyethylene glycol (PEG). After the incubation, the mineralized mesospheres were collected and analyzed by scanning electron microscopy (SEM). Mineralization with SBF resulted in spherical nanostructures with dimensions on the order of 100 nm patterned on the surface of PLGA mesospheres (FIGS. 22C and 22D). From SEMs and TEMs, the mineral nanostructures were distributed heterogeneously on the mesosphere surface, indicating formation of minerals at selected nucleation sites. It was observed that little or no mineralization occurred on the surface of NaOH-treated mesospheres when no quantum dots were incorporated into PLGA mesospheres (FIG. 24A). Thus, the presence of quantum dots was critical for the induction of mineralization on the spherical templates.

Without being bound by any particular theory, it is thought that several properties of quantum dots could potentially promote mineralization: (1) the physical presence of quantum dots (5 nm) could provide nanostructured templates on the surface of mesospheres to promote nucleation; (2) the ZnS shell of the CdSe core nanocrystal could provide the electrostatic and chemical interaction, which can trigger nucleation through increasing local supersaturation of ions and/or lowering the activation energy required for nucleation; and (3) residual TOPO, containing phosphine oxide, from the quantum dot solution can be present on the surface of mesospheres and promote mineralization by interacting with Ca²⁺. To test these hypotheses, quantum dots were replaced with either silver (10 nm) or gold (5 nm) colloids. No apparent mineral nanostructures were formed on the surface of PLGA mesospheres decorated with either silver or gold nanoparticles (FIGS. 24B and 24C). Incorporation of varying concentrations of TOPO in PLGA mesospheres did not result in mineralization on the surface of PLGA mesospheres either (FIG. 24D). These results suggest that quantum dots possessed a unique combination of chemical and physical features, which synergistically facilitate mineralization on the polymer mesospheres under physiological conditions.

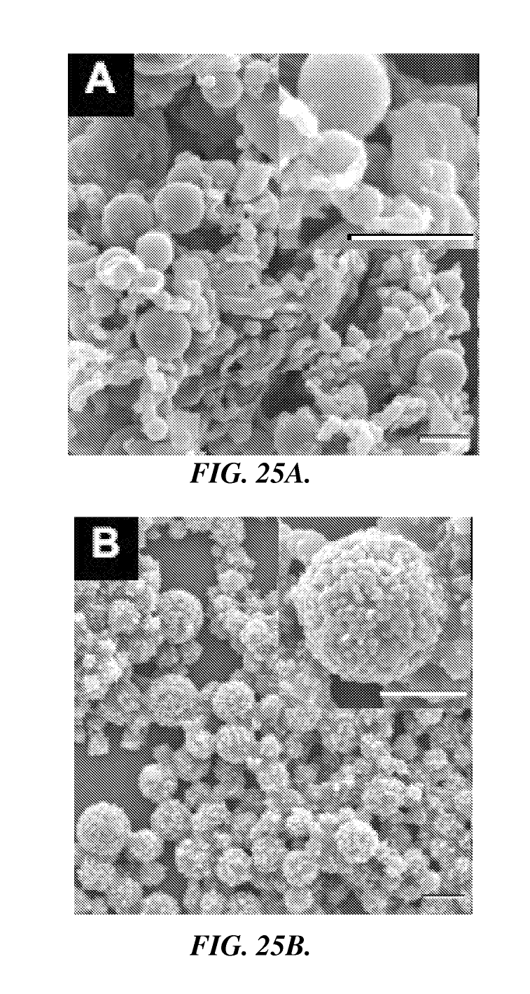

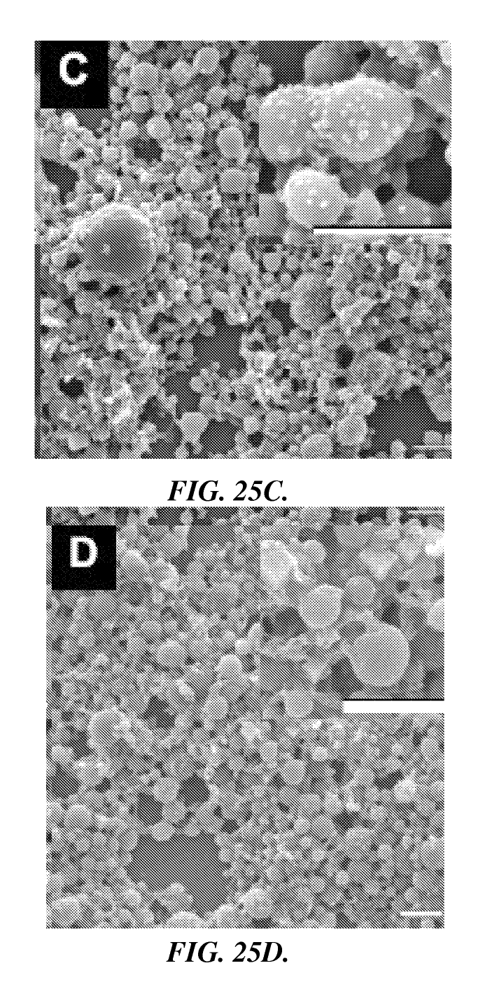

An additional challenge to pattern mineral nanostructures on the surface of individual mesospheres is that uncontrolled nucleation and growth are prone to occur in the solution. Polyethylene glycol (PEG, MW 4000) was added to suppress mineral growth in solution and confine mineralization on the surface of the mesospheres. It appeared that an optimal PEG concentration (0.2%) existed (FIG. 25B). Below the optimal concentration, mineralization dominantly occurred in solution but not on the spherical surface, resulting in aggregates of mesospheres and minerals (FIG. 25A); above the optimal concentration, mineralization is mostly inhibited in both solution and on the surface of mesospheres (FIGS. 25C and 25D).

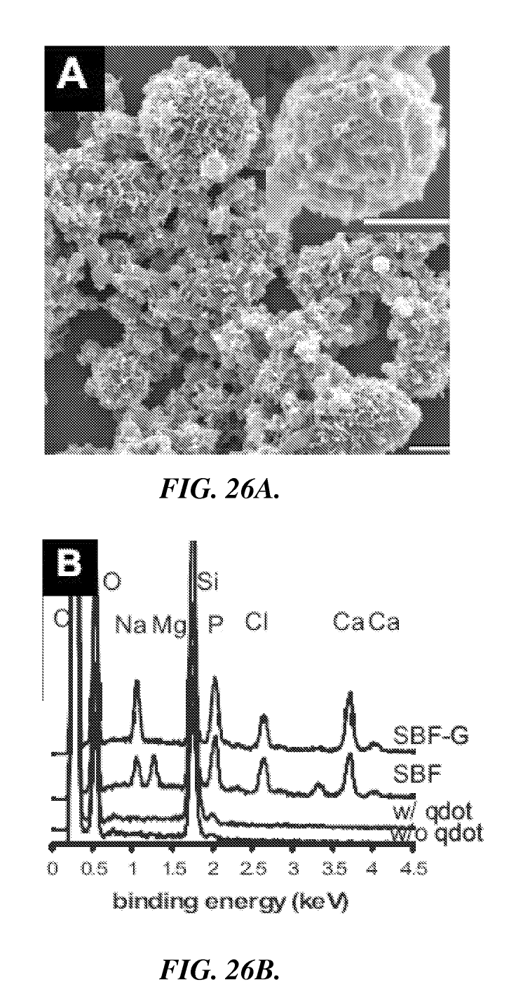

Lastly, it was considered whether various hierarchical morphologies could be patterned on PLGA mesospheres by tuning the mineral ion composition. Mineralization carried out with SBF-G, similar to SBF but lacking magnesium, resulted in thin, plate-like features (FIG. 26A) in contrast to spherical nanostructures formed with SBF (FIGS. 22C and 25B). Chemical analysis using elemental x-ray diffraction (EDX) demonstrated that the mineral nanostructures formed by SBF and SBF-G were mainly composed of calcium phosphate (FIG. 26B). Magnesium was detected on mesospheres mineralized with SBF but not SBF-G as expected. Therefore, by tailoring the composition of mineral solution used for mineralization, the morphology and chemical composition of minerals on the surface of mesospheres can be tuned. This programmability in both morphology and chemistry allows for the creation of hybrid nanomaterials towards a particular application, such as desirable tissue scaffolds to guide cell growth and differentiation, or drug delivery carriers capable of regulating intracellular environments to maximize the efficacy of delivered agents, and catalysts with high reactivity and selectivity.

This example describes a simple and versatile method for fabricating an inorganic shell-organic core hybrid material exhibiting hierarchical levels of organization and tunability, leading to a wide spectrum of potential applications.

### Example 5

### Environment-Responsive Nanocomposites for Intracellular Delivery of Biological Agents

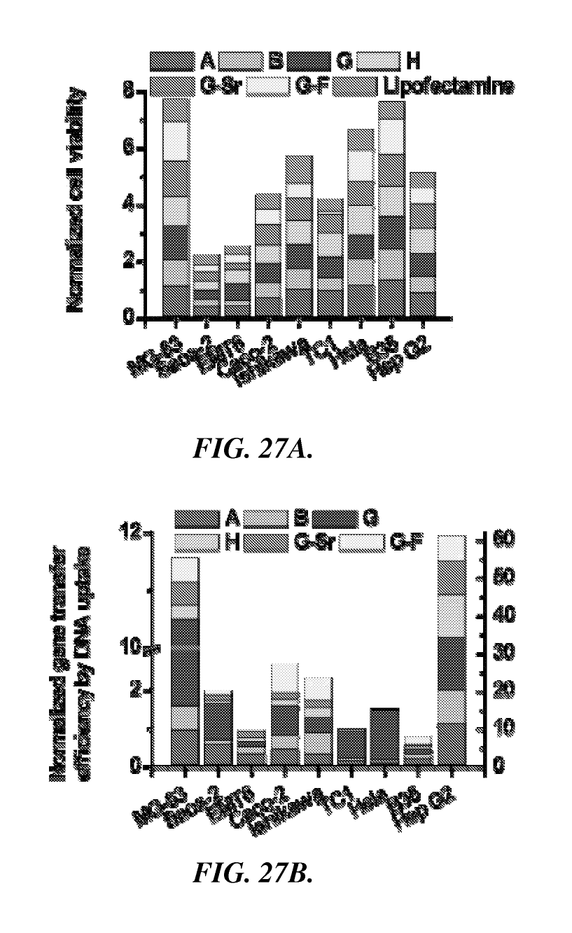

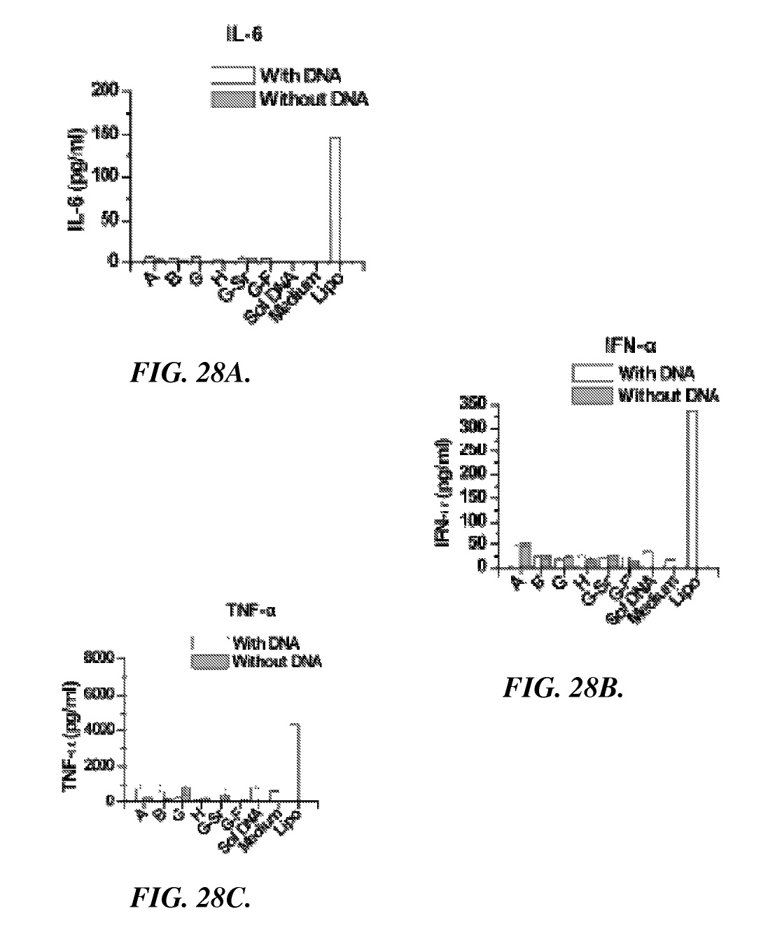

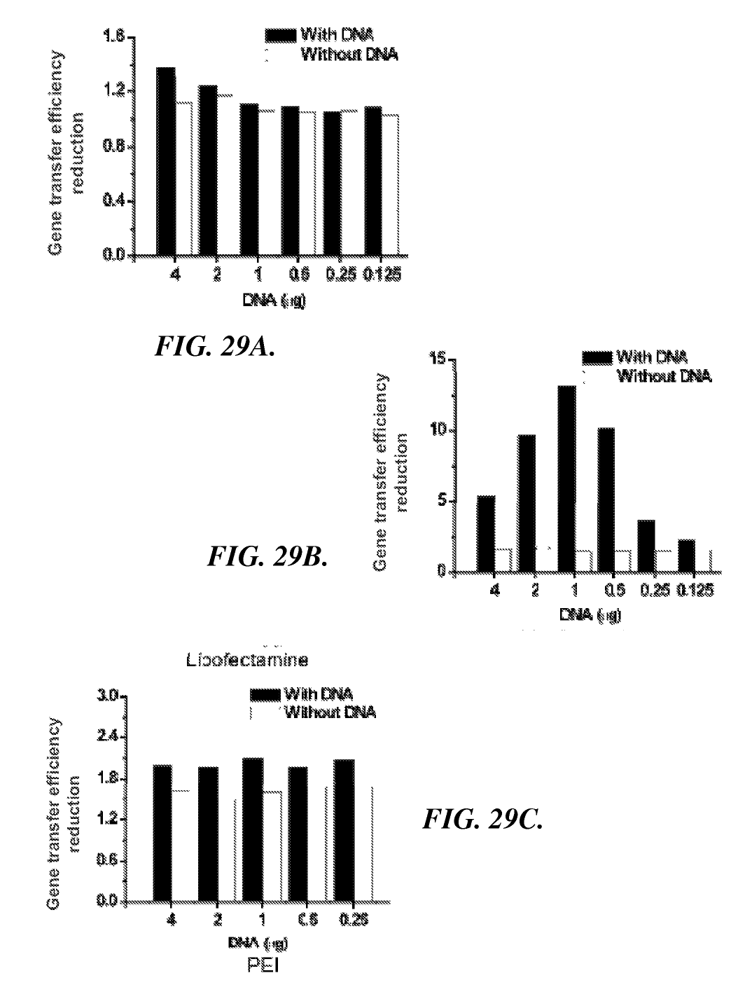

A platform based on environment-responsive nanocomposites, which exhibits low toxicity and tunable gene transfer in more than nine cell types is described herein (FIGS. 27A and 27B). Immunogenicity has also been tested by examining the secretions of inflammatory cytokines (i.e., IL-6, IFN-α, TNF-α) by macrophage cells exposed to nanocomposites. Nanocomposites induced negligible levels of inflammatory cytokines compared to Lipofectamine (Invitrogen) (FIGS. 28A-28C). The presence of these cytokines drastically decreases the expression level of transgene products over time in vivo. It was considered whether cytokines secreted from macrophages exposed to the platform of the present disclosure, Lipofectamine or Polyethyleneimine (PEI) would reduce gene expression. Cytokines collected from macrophages exposed to our platform did not reduce the gene expression level in lung epithelial cells (TC-1) while those exposed to Lipofectamine and PEI did (FIGS. 29A-29C).

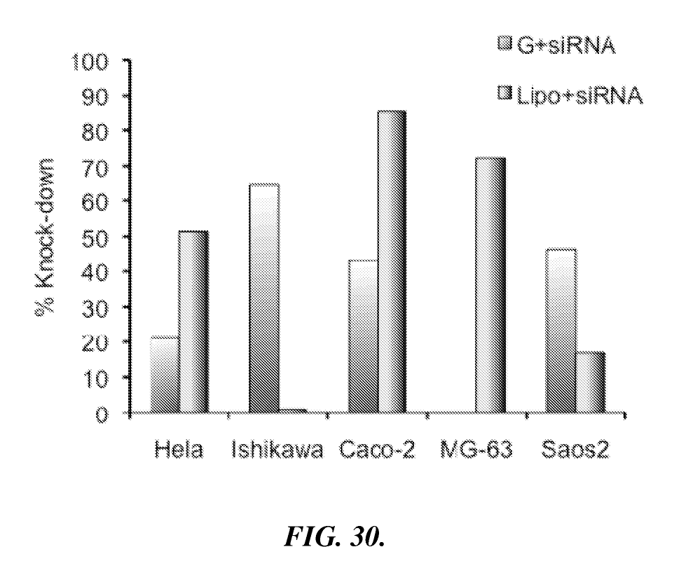

In comparison to similar products, the presently described platform offers several advantages including: ease of adapting to a specific clinical application (such as targeted drug delivery, vaccines), ease of manufacturing at a large scale, and low toxicity. This platform can be used to deliver other biologics, such as siRNA (FIG. 30), chemotherapeutic drugs, and microbicides as well.

Due to the strong correlation between material compositions with both gene transfer efficiency and immunogenicity, the presently described composition of nanocomposites can be tuned to optimize the gene transfer efficiency in a desired cell population and minimize the immunogenicity.

While illustrative embodiments have been illustrated and described, it will be appreciated that various changes can be made therein without departing from the spirit and scope of the invention.

1. A colloid, comprising a plurality of substantially spherical particles, each particle comprising a strontium-containing mineral component having a therapeutic agent dispersed therein.

2. The colloid of claim 1, wherein the strontium-containing mineral component has a ratio of strontium to calcium (Sr:Ca) is from about 0.2 to about 3.0.

3. The colloid of claim 1, wherein the strontium-containing mineral component has a (Sr+Ca)/P value of from about 1.5 to about 2.0.

4. The colloid of claim 1, wherein the strontium-containing mineral component has a ratio of strontium to phosphorus (Sr:P) is from about 0.3 to about 1.5.

5. The colloid of claim 1, wherein the strontium-containing mineral component has a ratio of calcium to phosphorus (Ca:P) is from about 0.5 to about 1.0.

6. The colloid of claim 1, wherein the strontium-containing mineral component has a percent crystallinity from about 80 to about 90%.

7. The colloid of claim 1, wherein the strontium-containing mineral component has an orientation of crystal growth along the hydroxyapatite 211 plane up to about 90%.

8. The colloid of claim 1, wherein the therapeutic agent is a biopolymer.

9. The colloid of claim 1, wherein the therapeutic agent is a biopolymer selected from the group consisting of a nucleic acid, a polysaccharide, a peptide, a protein, and fragments thereof.

10. The colloid of claim 1, wherein the therapeutic agent is a biopolymer selected from the group consisting of a DNA and an RNA.

11. The colloid of claim 1, wherein the therapeutic agent is a therapeutic small molecule.

12. The colloid of claim 1, wherein the therapeutic agent is a therapeutic small molecule is selected from the group consisting of a chemotherapeutic agent and an antimicrobial agent.

13. A pharmaceutical composition comprising a pharmaceutically acceptable carrier and a colloid of claim 1.

14. A method for delivery of a therapeutic agent to cell, comprising contacting a cell with the colloid of claim 1.

15. A method for regulating the expression of a gene or gene product, comprising contacting a cell with the colloid of claim 1, wherein the therapeutic agent is a biopolymer selected from the group consisting of a DNA and an RNA effective to regulate the expression of the gene or gene product.

16. A method for tissue engineering, comprising contacting cells with the colloid of claim 1, wherein the therapeutic agent is a nucleic acid or a protein.

17. A method for making a colloid, comprising:
(a) combining an aqueous strontium solution with a simulated body fluid to provide a strontium-containing mineralizing solution;
(b) combining the strontium-containing mineralizing solution with an aqueous therapeutic agent solution and an aqueous mineralization-inducing agent solution to provide a therapeutic agent-containing mineralizing solution; and
(c) maintaining the therapeutic agent-containing mineralizing solution at a temperature and for a period of time sufficient to effect mineralization to provide a strontium-containing colloid comprising a strontium-containing mineral component having the therapeutic agent dispersed therein.

18. The method of claim 17 further comprising collecting the strontium-containing mineral component.

19. The method of claim 18 further comprising re-suspending the strontium-containing mineral component in an aqueous medium to provide a second strontium-containing colloid.

20. The method of claim 17, wherein the simulated body fluid comprises ions selected from the group consisting of calcium ions, potassium ions, sodium ions, phosphate ions, chloride ions, and mixtures thereof.

21. The method of claim 17, wherein the aqueous therapeutic agent solution comprises a biopolymer.

22. The method of claim 21, wherein the biopolymer is a DNA or an RNA.

23. The method of claim 17, wherein the aqueous therapeutic agent solution comprises a therapeutic small molecule.

24. The method of claim 17, wherein the aqueous mineralization-inducing agent solution comprises a polyethylene glycol.

25-78. (canceled)
